SEQUENCE LISTING (12 sequences)

SEQ ID NO: 1 - DNA, 19 bp, Artificial sequence
  Synthetic oligonucleotide
gccagacacctgacactga

SEQ ID NO: 2 - DNA, 19 bp, Artificial sequence
  Synthetic oligonucleotide
tccgtcgttggtggtgatg

SEQ ID NO: 3 - DNA, 27 bp, Artificial sequence
  Synthetic oligonucleotide
tttccagacaaattccacctagagccc

SEQ ID NO: 4 - DNA, 19 bp, Artificial sequence
  Synthetic oligonucleotide
ggcgggaagactctggtta

SEQ ID NO: 5 - DNA, 19 bp, Artificial sequence
  Synthetic oligonucleotide
gtgccaggaaccaaaccta

SEQ ID NO: 6 - DNA, 24 bp, Artificial sequence
  Synthetic oligonucleotide
aagcaccgtggctgatcttccaga

SEQ ID NO: 7 - DNA, 20 bp, Artificial sequence
  Synthetic oligonucleotide
gcaaactttgctttccctgg

SEQ ID NO: 8 - DNA, 22 bp, Artificial sequence
  Synthetic oligonucleotide
acttcgagaggtccttttcacc

SEQ ID NO: 9 - DNA, 27 bp, Artificial sequence
  Synthetic oligonucleotide
cagccccaaaatggttaaggttgcaag

SEQ ID NO: 10 - DNA, 20 bp, Artificial sequence
  Synthetic oligonucleotide
ggacttcgagcaagagatgg

SEQ ID NO: 11 - DNA, 18 bp, Artificial sequence
  Synthetic oligonucleotide
caggtctttgcggatgtc

SEQ ID NO: 12 - DNA, 20 bp, Artificial sequence
  Synthetic oligonucleotide
tccttcctgggcatggagtc

(19) United States
(12) Patent Application Publication
(10) Pub. No.: US 2011/0287951 A1
(43) Pub. Date: Nov. 24, 2011

(54) METHODS AND SYSTEMS FOR PURIFYING, TRANSFERRING, AND/OR MANIPULATING NUCLEIC ACIDS

(76) Inventors: Michael R. Emmert-Buck, Easton, MD (US); Michael Armani, Olney, MD (US); Elisabeth Smela, Silver Spring, MD (US); Benjamin Shapiro, Washington, DC (US); Michael A. Tangrea, Odenton, MD (US); Jaime Rodriguez-Canales, Gaithersburg, MD (US); Rodrigo Chuaqui, North Potomac, MD (US); John Gillespie, Clarksville, MD (US)

(21) Appl. No.: 13/147,167

(22) PCT Filed: Jan. 29, 2010
(86) PCT No.: PCT/US10/22586

§ 371 Date: Jul. 29, 2011

Related U.S. Application Data
(60) Provisional application No. 61/206,458, filed on Jan. 30, 2009.

(51) Int. Cl. C40B 30/00; C12S 3/20; C07H 1/06; C12Q 1/68; C12P 19/34

(52) U.S. Cl. 506/7; 435/6.12; 435/91.2; 536/25.4; 435/270; 435/6.11

(57) ABSTRACT

The disclosure provides methods, systems, and devices for purifying, transferring or manipulating nucleic acids while maintaining the 2D spatial relationship of the nucleic acids as they were present in the original sample having 2D spatial information.

### CROSS REFERENCE TO RELATED APPLICATION

This application claims the benefit of U.S. Provisional Application No. 61/206,458, filed Jan. 30, 2009, which is incorporated by reference herein in its entirety.

### FIELD

This disclosure relates to the handling of nucleic acids, and more specifically to a device, system, and method for purifying, transferring, and/or manipulating nucleic acids from a biological sample without loss of spatial information.

### BACKGROUND

In the biological sciences and in clinical medicine there is a need for preserving and mapping the spatial distribution of nucleic acids at the tissue and cellular levels. For example, it is of importance to the understanding of embryonic development to know where, and in what concentration, particular molecules are found in specific cell types at various stages. Furthermore, in understanding disease it is useful to know which genetic mutations or alterations have occurred and where these changes have occurred within the tissue microenvironment. For instance, it can be desirable to know if changes occurred in the stroma or in the epithelium, and where the changes originated. Further, in diagnosing cancer at the earliest stages, it can be beneficial to know whether particular genetic alterations have occurred in a particular lesion such as a dysplastic cell population or pre-malignant focus.

Thus, there still exists an unmet need for techniques that allow the handling, manipulation, and/or analysis of biomolecules that maintains the two-dimensional (2D) spatial information of the source sample.

### SUMMARY

Disclosed herein are methods, systems, and devices for purifying, transferring, or manipulating nucleic acids from a sample, or performing a combination thereof, that substantially preserve two-dimensional (2D) spatial information on the original locations of the nucleic acids within the sample. Exemplary samples having 2D spatial information include, but are not limited to, a tissue section, an array of core samples from a specimen, an array of tissue-containing needles, an arrangement of cells adhering to a backing, a cell culture, a block of tissue, a tissue section encased in a gel or other matrix, nucleic acids contained within a gel or other matrix, a biopsy, or an organ. For example, planes through a three-dimensional (3D) biological sample, which may be exposed by sectioning, have 2D spatial information.

In general, a biological sample having 2D spatial information is provided to a platform with an array of chambers (which can comprise through-holes or a series of wells or vials), the sample is treated to free the nucleic acids, and the nucleic acids are transferred to the chambers. These methods, systems, and devices preserve the relative spatial locations of the nucleic acids by placing the sample in contact with the array of chambers and transferring the nucleic acids into the chambers by moving them in a direction perpendicular to the face of the sample into these chambers. After transfer, various manipulations and/or detection can be performed within these same chambers. The nucleic acids may further be transferred out of the chambers and into other platforms or onto other media for further manipulation and/or detection.

The foregoing and other features of the disclosure will become more apparent from the following detailed description of several embodiments which proceeds with reference to the accompanying figures.

### BRIEF DESCRIPTION OF THE FIGURES

FIG. 1 is a perspective schematic view of providing a tissue sample 20 to a manipulation platform (also referred to herein as a substrate) 10 having a plurality of chambers 30 while maintaining the relative spatial relationships of the nucleic acids in the original sample.

FIG. 2 is a cross-sectional schematic view of providing tissue sample 20 to manipulation platform 10 having plurality of chambers 30 while maintaining the relative 2D spatial relationships of the nucleic acids in the original sample.

FIG. 3A-3C provide a cross-sectional schematic view of transferring target nucleic acids from a sample having 2D spatial information 20 into chambers 30 while maintaining the relative 2D spatial relationships of the nucleic acids that were present in sample 20. FIG. 3A illustrates a pressure 41 as applied to a plate 40 over sample 20. As a result of pressure 41, one or more portions of tissue 21, each of which overlies one of chambers 30, are pushed into chambers 30, whereas at least some biological sample overlying areas between chambers 31 remains outside the chambers, resulting in excluded tissue 22. FIG. 3C is a schematic view of an apparatus for applying pressure to sample 20 to push tissue overlaying chambers into chambers 30. In the illustrated embodiment, the apparatus has a frame 150 to which a clamp 151 is secured, such as by a fastener (e.g., a screw) that allows the clamp to be manipulated, such as raising or lowering the clamp by rotation. A block 152 is adjacent to clamp 151 so that, when clamp 151 is lowered, block 152 comes into contact with a sealing film 153 which covers sample 20, which is in turn is contacting manipulation platform 10. Coupled to manipulation platform 10 is a thermocycler 155, which in turn is optionally coupled to a scale 156 for measuring pressure.

FIG. 4A is a cross-sectional close-up schematic view illustrating treating a transferred biological sample 21 overlying the chambers 30 with reagents 50 within chambers 30 to free nucleic acids 23 while maintaining the 2D spatial relationship of nucleic acids 23 to each other relative to the original sample.

FIG. 4B is a cross-sectional close-up schematic view illustrating freed nucleic acids 23 which are available for manipulation or detection after the treatment illustrated in FIG. 4A.

FIGS. 5A-5D provide a cross-sectional close-up schematic view of a series of vessels to illustrate an exemplary method of purifying nucleic acids 27 from a sample having 2D spatial information 20. The method includes providing a vessel 14 (FIG. 5A). The method also includes providing a nucleic-acid binding surface 94, biological sample 20 containing nucleic acids 27, and a lysis or digestion reagent 52 to vessel 14. As a result, nucleic acids 27 are released from sample 20 (FIG. 5B). The method also includes adding a nucleic acid precipitation agent 56 as illustrated in FIG. 5C. As a result, nucleic acids 27 are simultaneously released and bind to nucleic-acid binding surface 94. The method also includes removing unbound species from the vessel such as by rinsing, or suction by vacuum, of the fluid in the vessel. At this point, nucleic acids 27 are purified and bound to nucleic-acid binding surface 94 (FIG. 5D).

FIGS. 6A-6C provide cross-sectional close-up schematic views illustrating an exemplary method of treating a sample having 2D spatial information 20 to free nucleic acids 23 from sample 20 while maintaining the 2D spatial relationship of the nucleic acids relative to the original sample in accordance with the disclosure herein. FIG. 6A provides a cross-sectional close-up schematic view of sample 20 which is positioned on a layer of gel 61 and reagents (not shown) that digest the tissue matrix are added. Two-dimensional biological sample 20 is composed of cells 25 having nuclei 24 that contain target nucleic acids 23. FIG. 6B illustrates a cross-sectional close-up schematic view of sample 20 including a second layer of gel 62 which is added on top of sample 20, encasing it and preventing the components from moving. FIG. 6C provides a cross-sectional close-up schematic view of a sample following digestion, in which nucleic acids 23 are free inside a digested sample matrix 26.

FIG. 7 is a cross-sectional schematic view of digested sample matrix 26 with freed nucleic acids 23 sandwiched between two layers of gel 61, 62 to manipulation platform 10 having a plurality of chambers, which are through-hole micro-scale chambers 32, while maintaining the relative 2D spatial relationships of the nucleic acids in the original sample. Micro-scale chambers 32 are filled with a gel 60.

FIG. 8 is a cross-sectional schematic view of nucleic acids 23 that are freed from digested 2D tissue sample 26 encased in gel 61, 62 into manipulation platform 10 with plurality of through-hole micro-scale chambers 32 filled with gel 60 while maintaining the relative 2D spatial relationships of nucleic acids 23 in the original sample. A voltage source 70 applies a voltage via leads 71 to an anode 73 and a cathode 72 immersed in an electrolyte 74, creating an electric field. Nucleic acids 23 are charged and therefore move by electrophoresis under the electric field into through-hole micro-scale chambers 32. Nucleic acids 23 pass through gel 61, but other molecules do not, purifying the sample.

FIG. 9 is a cross-sectional schematic view illustrating transferring nucleic acids 23 that are free from digested sample matrix 26 encased in gel 61, 62 into manipulation platform 10 with a plurality of through-hole micro-scale chambers 32 while maintaining the relative 2D spatial relationships of nucleic acids 23 in the original sample. A suction force 45 pulls nucleic acids 23 into through-hole micro-scale chambers 32.

FIG. 10 is a cross-sectional schematic view illustrating transferring nucleic acids 23 that are free, such as from digested sample matrix 26 into manipulation platform 10 with a plurality of gel chambers 63 while maintaining the relative 2D spatial relationships of nucleic acids 23 as they were in the original 2D tissue sample (FIG. 10a). Nucleic acids 23 diffuse from digested sample matrix 26 into gel chambers 63 (as illustrated in the schematic on the right, FIG. 10b).

FIG. 11 is a cross-sectional schematic view of manipulation platform 10 comprising three substrates (10a, 10b, and 10c) having through-hole micro-scale chambers 32, which have been aligned or registered, on top of which is digested sample matrix 26 with nucleic acids 23 which have been freed. A first step of sample manipulation takes place in platform 10a, for example filtration or binding. This step is followed by transfer to substrate 10b, where another manipulation takes place, such as polymerase chain reaction. This is followed by transfer to substrate 10c, where a third manipulation step and/or detection take place, such as fluorescent tagging. The interior of the wells of the different substrates may contain different materials (60, 64, 65) to aid the different manipulation and detection procedures.

FIG. 12 is a cross-sectional schematic view of mapping the position of target nucleic acids within manipulation platform 10. A source of excitation light 80 shines light 81 (represented by arrows) onto the target molecules in manipulation platform 10. The target molecules have been tagged with a fluorescent dye. The chambers 30 containing target molecules emit fluorescent light 82 (represented by different arrows), which is detected by a light detector 83.

FIG. 13 is a schematic illustration of an exemplary method for creating a molecular map. Molecules are transferred (represented by arrow 104) from 2D tissue sample 20 to manipulation platform 10. Target nucleic acids are then amplified by PCR within the chambers 30. The amplification products are transferred out of manipulation platform 10 (indicated by arrow 105) and onto a membrane 90 for labeling (stain, radioisotope, or fluorescent dye) and detection. Labeled areas 92 on membrane 90 indicate the spatial locations and concentrations of the target nucleic acids.

FIG. 14A is a schematic illustration of the vertical transfer of nucleic acids out of chambers 30 in manipulation platform 10 and onto a stack of capture membranes (90b, 90c, 90d). Each membrane has been treated to capture a different target molecule, which is visualized by staining (92b, 92c, 92d).

FIGS. 14B-14D are schematics of hypothetical maps of the molecules trapped on membrane 90b (FIG. 14B), 90c (FIG. 14C) and 90d (FIG. 14D) after staining and overlaying the respective maps over an image of the original 2D tissue sample.

FIG. 15 is a schematic illustration of an exemplary embodiment in which the method of transferring nucleic acids, manipulating such nucleic acids followed by detecting the manipulated nucleic acids. First, nucleic acids 23 are transferred vertically 100 (represented by the arrow) from electrophoresis gel 65 into manipulation platform 10. For illustration, three samples containing nucleic acids 23 have been loaded onto three lanes of gel (66a, 66b, 66c), and nucleic acids 23 separated on the gel, for example by size using electrophoresis. Nucleic acids are manipulated 102, for example by PCR, in the manipulation platform 10 on three target nucleic acids. The products are transferred 101(represented by the arrow) to a stack of capture membranes (90b, 90c, 90d), each of which is treated to capture a different target nucleic acid. The positions of the nucleic acids can be visualized by staining 92.

FIGS. 16A-16C provide a series of digital images of exemplary substrate in having micro-scaled chambers. FIG. 16A is a digital image of an array of micro-scale wells etched into a silicon wafer. FIG. 16B is a close-up overhead view of a micro-scale well with a 500 μm opening at the top surface etched by anisotropic wet etching into a silicon wafer. FIG. 16C is a scanning electron microscope cross-sectional image showing micro-scale wells approximately 100 μm in diameter etched by deep reactive ion etching into a silicon wafer.

FIG. 17A is an overhead view digital image of an array of micro-scale wells 30 etched into a silicon wafer by deep reactive ion etching in accordance with an embodiment of the present disclosure.

FIG. 17B is a digital image of a cross-sectional view of the array of micro-scale wells 34 etched into a silicon wafer by deep reactive ion etching shown in FIG. 17A.

FIG. 18A is a digital image of an overhead view of an array of millimeter-scale through-holes drilled into an aluminum sheet.

FIG. 18B is a digital image of an oblique view of a 14 μm thick section of dried human prostate tissue on a platform with an array of millimeter-scale chambers.

FIG. 19A is a schematic diagram showing the placement of positive controls 110 (white circles), negative controls 111 (black circles), and dye 112 (crosshatched circles) for registration into manipulation platform 10 with mm-scale through-hole style chambers 33.

FIG. 19B is a digital image of a 100×SYBR-gold/nitrocellulose membrane visualization of the results of PCR experimentally carried out inside the mm-scale chambers with the placement of the samples according to FIG. 19A.

FIG. 20 is a schematic diagram of a registration pattern 120 (black) imprinted onto sample 20 to allow later registration of histology and molecular maps.

FIG. 21A is a digital image of an overhead view of an aluminum manipulation platform 11 with through-hole style chambers 30. Overlying the surface is sample 20, which is a tissue section that has been stained. After transfer of the DNA of the tissue section into chambers 30, the DNA was freed from the tissue, a target sequence was amplified by PCR, and the amplification products were manipulated 102, the set of manipulations is represented by the arrow.

FIG. 21B is a digital image of chambers 33 containing the amplification product illustrated by the emission of fluorescent light 82. The outline of a tissue sample 20a as it was originally placed on the surface is indicated. FIGS. 21A and 21B illustrate that target nucleic acids can be provided to a manipulation platform, freed from the sample, and transferred into the platform while maintaining the 2D spatial relationship of the transferred material relative to the original sample, and can then subsequently be manipulated and detected while still maintaining the original spatial relationship they had in the 2D tissue sample.

FIG. 22A is a digital image of the same fluorescence image provided in FIG. 21B showing the location of recovery sites for post-PCR validation of product amplification. Labels 1-6 correspond to positive detection of tissue genomic DNA targets and labels 7-12 correspond to negative detection.

FIG. 22B is a digital image of a 2.5% agarose electrophoresis gel showing the post-PCR validation of products. The number designation of the lanes corresponds to the designation of items in FIG. 22A.

FIG. 23 is a digital image of three identical rows of a platform with mm-scale wells with, left to right, FAM reporter of CT values 24 (130), negative, 29.7 (131), 49.2 (132), negative (133), 48.8 (134), and ROX background stain (135).

FIGS. 24A-24C are digital images of an embodiment of the disclosure following addition of TaqMan in 100 μm micro-wells, looking at different areas on one platform. FIG. 24A is a digital image showing strong fluorescence with a positive control with CT value of 24; FIG. 24B, negligible fluorescence with a negative control (no CT value); and strong fluorescence with a positive control with CT value of 29.69.

FIGS. 25A-25E are cross-sectional close-up schematic views of an exemplary embodiment of a method of purifying nucleotides from a sample having 2D spatial information. FIG. 25A illustrates providing vessel 14 and providing nucleic-acid binding surface 94 to vessel 14. In this example, vessel 14 comprises a substrate with an array of through-hole chambers 33, the through-holes designed to have a volume for the containment of nucleic-acid binding surface 94 and reagents that opens via holes 140, which are approximately the same diameter as the diameter of the containment volume, having a large surface area to volume ratio, onto the top face and holes 141, which are smaller than the diameter of the containment volume and sufficiently small to hold the nucleic-acid binding surface 94 within the vessel, onto the bottom face for the removal of fluids and the containment of nucleic-acid binding surface 94. Vessel 14 further includes a first reversible seal 16a to close holes 141 on the bottom face. FIG. 25B shows adding into vessel 14 sample 20 containing nucleic acids 27 and protein denaturing agent 52. Nucleic acid precipitation agent 56 is added at the same time. FIG. 25C illustrates allowing sufficient time to elapse to free the nucleic acids from the biological sample. In this example, the openings on the top surface of vessel 140 are closed with a second reversible seal 16b to prevent evaporation. Nucleic acids 27 are released from biological sample 20 and bind to nucleic-acid binding surface 94. FIG. 25D illustrates removing unbound species from the vessel. In this example, this is done by applying vacuum suction to the bottom of through-hole chambers 33. At this point, nucleic acids 27 are left purified and adhered to nucleic-acid binding surface 94. FIG. 25E illustrates adding blocking agent 54 to vessel 14, which binds to nucleic-acid binding surface 94 and releases nucleic acids 27 from nucleic-acid binding surface 94. At this point, nucleic acids 27 are available for subsequent manipulation, analysis, amplification, or detection.

FIGS. 26A and 26B are cross-sectional close-up schematic views of the formation of array of vessels 14 by placing a sealing film 16c onto the bottom face of an array of through-hole chambers 33 (as illustrated in FIG. 26A) and punching holes into sealing film 16c at the centers of through-hole chambers 33, producing a bottom surface 16d of vessel 14 with apertures for allowing the draining of fluid while containing nucleic-acid binding surfaces (as illustrated in FIG. 26B).

FIG. 27 is a cross-sectional close-up schematic view of nucleic acid binding material 94 provided to vessel 14 by placing it against a face of the vessel.

FIG. 28 is a flow chart showing a comparison of the workflow for RNA extraction, purification, and detection between an RNase inhibitor based method and the disclosed methods. Optional steps are indicated by boxes with dashed lines.

FIG. 29 is a flow chart showing a comparison of the workflow for RNA extraction, purification, and detection between a phase separation-based method and the disclosed methods. Optional steps are indicated by boxes with dashed lines.

FIG. 30 is a flow chart showing a comparison of the workflow for RNA extraction, purification, and detection between the QIAGEN® RNeasy® (both registered trademarks of QIAGEN Group) silica filter based method and the disclosed methods. Optional steps are indicated by boxes with dashed lines.

FIG. 31 is a flow chart showing a comparison of the workflow for RNA extraction, purification, and detection between the Molecular Devices PICOPURE® (a registered trademark of Molecular Devices) silica filter based method and the disclosed methods. Optional steps are indicated by boxes with dashed lines.

FIG. 32 is a flow chart showing a comparison of the workflow for RNA extraction, purification, and detection between an oligo-dT magnetic bead-based method and the provided method. Optional steps are indicated by boxes with dashed lines.

FIGS. 33A and 33B are digital images of typhoon imager results illustrating detection from tissues and control mRNA (FIG. 33A) and verification of mRNA in the tissue sample (FIG. 33B).

FIGS. 34A and 3B are digital images of detection of positive and negative mRNA containing samples by TaqMan one-step PCR (FIG. 34A) or detection of target 120 nt product corresponding to positive fluorescent TaqMan samples (FIG. 34B).

FIG. 35 is a digital image of liver sections transferred onto a 384-well plate. The three sections are located within the area indicated by dashed black circles. The vials over which they lay are indicated by the white outline.

FIG. 36 is a digital image of PCR products separated by electrophoresis generated following transferring the tissues provided in FIG. 35.

### SEQUENCE LISTING

The nucleic and/or amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

### DETAILED DESCRIPTION OF SEVERAL EMBODIMENTS

### I. Introduction

Disclosed herein are methods, systems, and devices for purifying nucleic acids from a sample having 2D spatial information and for transferring nucleic acids from a sample having 2D spatial information to a manipulation platform (e.g., a substrate having a plurality of chambers), which are beneficial because they maintain the 2D spatial positions of the molecules in the original sample, thereby permitting subsequent manipulation of the molecules in a robust manner without interference from components of the tissue. The disclosed methods, systems, and devices can be used with molecules present at low abundance and to rapidly extract the target molecular information from a biological sample, such as a tissue sample. These methods, systems, and devices can preserve the spatial positional information so that it can be correlated with specimen information. Exemplary methods, systems, and devices are disclosed that can preserve the spatial positional information at the micro-scale level so that it can be correlated with specimen information at the level of tissue type or cellular level. Further, the disclosed methods, systems, and devices can be utilized to obtain the target molecular information from an entire tissue sample without selection bias, to deliver highly purified molecular samples to an engineered matrix, allowing subsequent reactions to proceed robustly and allowing highly sensitive detection methods to be applied. These methods and devices can be used with multiple samples, including non-genetically modified organisms and any type of tissue. In certain embodiments, the methods and devices not only facilitate the transfer of nucleic acids into a manipulation platform, but also the transfer of nucleic acids out of the manipulation platform after manipulations have taken place for detection while maintaining the 2D spatial positions of the molecules in the original sample, thereby allowing the creation of molecular maps.

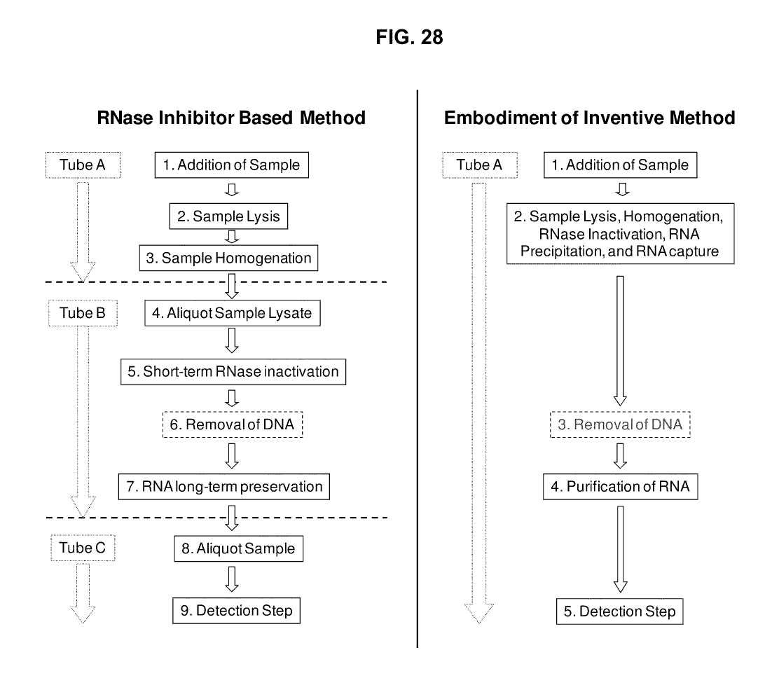

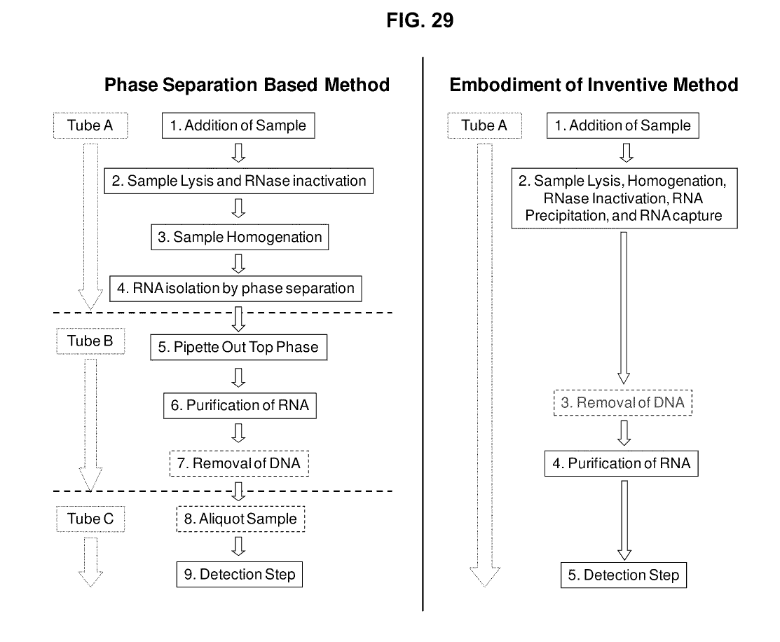

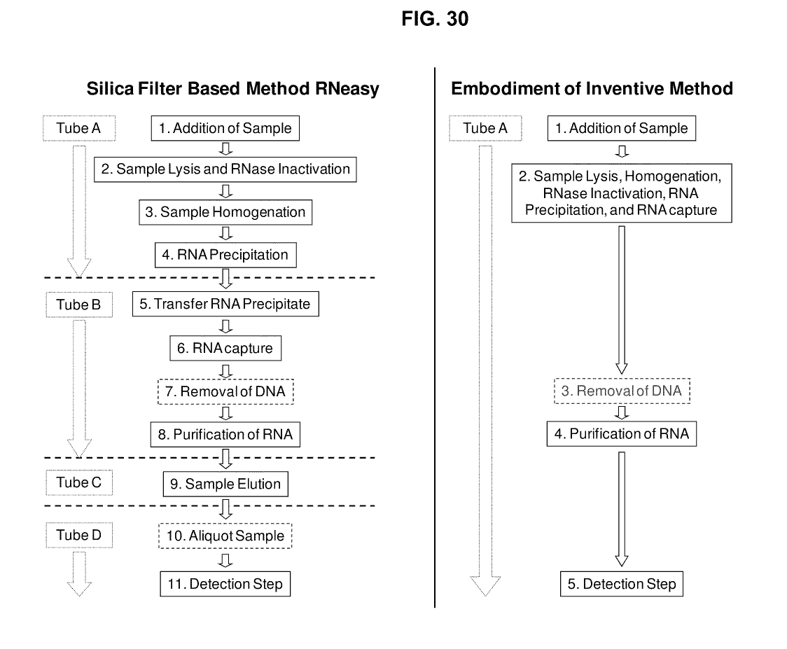

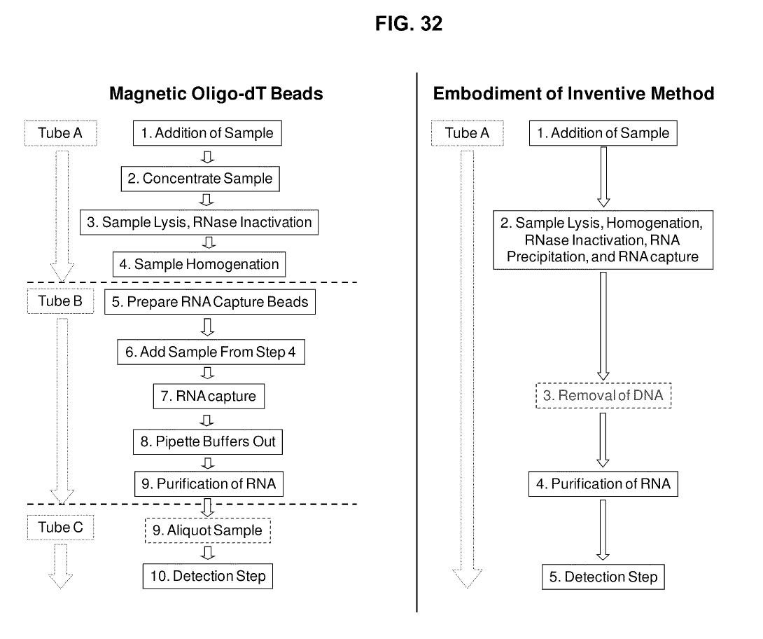

The methods of purifying nucleic acids disclosed herein can allow for the purification of nucleic acids in a single vial, well, chamber, vessel, “tube”, or patch of gel. For example, nucleic acids can be extracted from tissues in a single vessel, enabling downstream amplification of the extraction nucleic acids within the same vessel. In one specific embodiment, not only can nucleic acids be purified in a single vessel by use of the disclosed methods, systems, and devices, but they can in addition be manipulated and detected while only using a single vessel during the entire procedure, while the current art teaches away from using a single vessel for an entire procedure by suggesting two vessels for extraction and purification, and a third vessel for detection. Thus, the methods, systems, and devices provided herein can allow RNA to be purified more quickly and consistently than current methods (see for comparison FIGS. 28-32), with surprisingly greater yields, and these methods can be used with a wider range of tissue types and they allow higher throughput. For example, nucleic acids can be purified and manipulated with minimal handling and a minimal number of steps, and are amenable to miniaturization or robotic handling. In particular, the methods, systems, and devices for the purification of RNA disclosed herein allow for, lysis of cells, extraction of RNA, inactivation of RNases, and precipitation of RNA onto a binding surface to occur simultaneously within a single vessel, in contrast to the current methods that require selective pipetting of particular phases out of the reaction vessel.

### II. Overview of Several Embodiments

Disclosed herein are methods, systems, and devices for transferring and extracting nucleic acids from a biological sample, such as a tissue sample, that allow one or more of nucleic acid purification, amplification, and detection without loss of 2D spatial information. In one embodiment, a method for transferring, purifying, and amplifying nucleic acids from a sample having 2D spatial information within a substrate while maintaining the 2D spatial relationship between the nucleic acids that were present in the original sample is provided. The method includes providing the sample to the substrate, which substrate includes a substrate having a plurality of chambers, such as through-holes, wherein each through-hole comprises a first opening on a first face of the substrate and a second opening on a second face of the substrate thereby forming a through-hole. In certain embodiments, the smaller the diameter of the chamber, the higher the resolution of that can be achieved in locating the spatial positions of the nucleic acids. However, as their size shrinks, it can be difficult to transfer material into the chambers by traditional means like pipetting. Also, other physical effects, such as surface tension, may play an increasingly larger role in such situations. For example, surface-to-volume ratios may be considered at smaller scales, since non-specific binding on the chamber wall surfaces may affect reactions, manipulations, or detections.

For example, the sample having 2D spatial information is provided to the substrate by contacting the sample to the first surface of the substrate. Exemplary samples having 2D spatial information include a tissue section, an array of core samples from a specimen, an array of tissue-containing needles, an arrangement of cells adhering to a backing, a cell culture, a block of tissue, a tissue section encased in a gel or other matrix, nucleic acids contained within a gel or other matrix, a biopsy, or an organ. Such methods also can include transferring portions of the sample having 2D spatial information into the plurality of chambers of the substrate and providing conditions sufficient to free nucleic acids from the transferred tissue portions within the plurality of chambers of the substrate. For example, providing conditions sufficient to free nucleic acids can include performing a digestion (such as treatment with proteinase K, or trypsin), inactivation of the digestion, a denaturation of nucleic acids or proteins, a purification of free nucleic acids or proteins, or a combination of two or more thereof. In an example, transferring portions of the sample into the plurality of chambers of the substrate include applying pressure or suction to the sample to express portions of the sample into the plurality of chambers in the substrate.

In some embodiments of the method, the method further includes treating the substrate with agarose prior to providing the sample to the substrate. In certain embodiments, the method includes applying a sealing material to the second face of the substrate prior to providing the sample having 2D spatial information to the substrate. In one embodiment, the method includes adding a registration mark to the 2D tissue sample prior to transferring portions of the 2D tissue sample to the substrate.

In some embodiments, the method further includes amplifying target molecules, such as nucleic acids, by PCR in the presence of blocking agent (such as bovine serum albumin) and amplification reagents, wherein the blocking agent is added prior to the amplification reagents and comprises about 0.1% to about 1% of the total volume of an amplification reaction, thereby allowing amplification of target nucleic acids while preserving the 2D spatial relationship of the target nucleic acids relative to their original position in the original 2D tissue sample throughout the method in a substrate.

In one embodiment, the method includes detecting a pre-determined characteristic of the target nucleic acids using a 2D spatial map of the predetermined characteristic. In some embodiments, the method also includes creating cDNA from mRNA prior to performing the polymerase chain reaction. In particular embodiments, the method further includes detecting the target nucleic acids.

Other embodiments include methods for loading target nucleic acids in a sample having 2D spatial information into a substrate having a plurality of micro-scale chambers while maintaining the 2D spatial relationship between the nucleic acids that were present in the original sample is disclosed. In one embodiment, the method of loading includes providing the sample to the substrate including the plurality of micro-scale chambers; transferring portions of the sample into the plurality of micro-scale chambers; and providing conditions sufficient to free nucleic acids from the transferred tissue portions within the plurality of micro-scale chambers. In the disclosed method of loading, the nucleic acids are placed into an aqueous environment that allows subsequent manipulation, detection, or combination thereof of nucleic acids while maintaining the 2D spatial relationship of the nucleic acids relative to those in the original biological sample throughout the method in the substrate.

Exemplary samples having 2D spatial information include a tissue section, an array of core samples from a specimen, an array of tissue-containing needles, an arrangement of cells adhering to a backing, a cell culture, a block of tissue, a tissue section encased in a gel or other matrix, nucleic acids contained within a gel or other matrix, a biopsy, or an organ. In one example, providing the sample includes contacting the sample to a first surface of the substrate. In some examples, providing conditions sufficient to free nucleic acids comprises cell lysis, a digestion of proteins (such as with proteinase K or trypsin), an inactivation of the digestion, a denaturation of nucleic acids, a purification of free nucleic acids, or a combination of two or more thereof.

In some embodiments, the disclosed method for loading target molecules further includes amplifying nucleic acids (e.g., by using polymerase chain reaction, rolling circle amplification, loop-mediated amplification, helicase dependent amplification, or ligation chain reaction) and/or detecting nucleic acids (e.g., by fluorescence). Other manipulations may include desiccation or drying for storage, rinsing the wells, heating the wells, a binding, inactivation, denaturation, degradation, release from binding, labeling, and combinations thereof.

In further embodiments, disclosed herein is a method for purifying nucleic acids from a biological sample, such as a tissue sample, within a single vessel. This method can be used with other methods disclosed herein as well as the devices provided herein. However, this method can also be used with any device, system, or method depending upon the needs of the user (such as for robotic automation). In one example, the method includes providing a vessel including polypropylene, polyethylene, polystyrene, polycarbonate, fluoropolymer, acrylic, aluminum, stainless steel, ceramic, silicone, silicon, glass, quartz, acrylic adhesive resin, silicone adhesive resin, surfaces made compatible for PCR with a biocompatible surface coating such as poly-ethylene-glycol as is known in the art, or a combination of two or more thereof. The method also includes providing a nucleic-acid binding surface to the same single vessel, the nucleic-acid binding surface comprising silica, silicon, silicon carbide, silicon nitride, metal oxides, polycarbonate, polystyrene, nitrocellulose, cellulose, or chitosan. In one particular example, the nucleic-acid binding surface is a silica filter, silica beads, or silica powder. The surface area of the nucleic acid binding surface is of sufficient size to bind the freed nucleic acids with substantial efficiency. The larger the area of the binding surface, the more of the nucleic acids in the solution that can be bound, and therefore the more nucleic acid that can be purified, and thus the more nucleic acid that is available for subsequent manipulation or detection.

The methods also can include adding a blocking agent to the vessel to allow subsequent detection in the same vessel. Exemplary blocking agents can include bovine serum albumin, polyethylene glycol, polyvinylpyrrolidone, Tween 20, or a combination thereof. In one example, heat can be applied during the liberation of nucleic acids. The disclosed method also includes precipitating nucleic acids using a precipitation agent and removing unbound species from the vessel, thereby purifying nucleic acids from a biological sample in a single vessel. For example, the unbound species can be removed from the vessel by rinsing (e.g., flushing the vessel with solutions sufficient to wash away the contents of the vessel other than non-degraded nucleic acids). In a further example, a binding surface comprises a material which has a pH-dependent surface charge, and the blocking agent changes the charge (from positive to negative or vice versa) to release bound nucleic acids.

The method further includes using surfaces having oligonucleotides, protein nucleic acids, or locked nucleic acids as the binding surface in the single vessel. In an example, added into the same single vessel are a tissue sample, oligo-dT magnetic beads, at least about 25% by volume guanidinium isothiocyanate and at least about 11% by volume Triton X-100, said protocol utilizing a precipitation step which includes adding water, which surprisingly leads to a 4-fold improvement over prior protocols. In another embodiment, the method can include a vortexing step to sufficiently lyse tissues to free the nucleic acids from a tissue. The method also includes a rinsing step.

In certain embodiments, the desired nucleic acid to purify is RNA, and DNase is added to degrade DNA. In some examples, the method further includes amplification or detection of the nucleic acids in the same single vessel following purifying the nucleic acids.

In one example, the method of purifying nucleic acids includes a single vessel with two openings, and the fluid can be flushed through the vessel, such as by means of mechanical forces (e.g., pressure differential). In some embodiments, the method further includes sealing the vessel to help reduce evaporation of the fluids.

In further embodiments, provided herein is a system for preserving the 2D spatial positions of target nucleic acids relative to their original position within a 2D tissue sample. In one embodiment, the system includes a substrate having a plurality of through-holes, wherein each through-hole includes a first opening on a first face of the substrate and a second opening on a second face of the substrate thereby forming a through-hole, (such as a through-hole with a diameter of 50 μm to 150 μm) wherein the nucleic acids can be held, manipulated, or detected. In one example, the substrate further includes agarose within the through-holes.

This system also includes a mechanism for transferring the nucleic acids from the sample having 2D spatial information into the plurality of chambers while maintaining the 2D architecture of the transferred nucleic acids relative to their position in the original sample, whereby the nucleic acids are placed into an aqueous environment that allows preservation, manipulation, and/or detection. In some embodiments, the system further includes a tissue section, an array of core samples from a specimen, an array of tissue-containing needles, an arrangement of cells adhering to a backing, a cell culture, a block of tissue, a tissue section encased in a gel or other matrix, nucleic acids contained within a gel or other matrix, a biopsy, or an organ.

In one embodiment, the mechanism for transferring the nucleic acids can be electrophoresis, which can make use of an anode, a cathode, electrical leads connecting the anode and cathode to an electrical power supply, and a housing for containing electrolyte. The anode and cathode are in contact with the electrolyte, whereby an electric field is created between the anode and cathode, causing movement of charged nucleic acids out of the tissue sample and into the plurality of through-hole style chambers. In other examples, the transfer of the nucleic acids is facilitated by pressure or suction.

### III. List of Reference Numerals Used in the Figures

10 manipulation platform or substrate
10a a first substrate of the manipulation platform
10b a second substrate of the manipulation platform
10c a third substrate of the manipulation platform
11 manipulation platform made of Al
14 vessel
16a first reversible seal
16b second reversible seal
16c sealing film
16d bottom surface formed by sealing film of 16c
20 2D tissue sample or biological sample
20a outline of tissue sample or biological sample position
21 tissue or biological sample overlying the chambers
22 tissue or biological sample overlying the areas between chambers; also referred to as excluded tissue
23 nucleic acids
24 nuclei
25 cell
26 digested sample matrix
30 chambers or wells
31 areas between chambers
32 through-hole style micro-scale chambers
33 through-hole style mm-scale chambers
34 micro-scale chambers
40 plate
41 pressure
45 suction force
50 reagents that digest tissue matrix
52 protein denaturing agent
54 blocking agent
56 nucleic acid precipitation agent
60 gel within the manipulation platform 10
61 gel encasing the tissue, bottom layer
62 gel encasing the tissue, top layer
63 chambers made of gel
64 a material to aid manipulation or detection within the wells of a manipulation platform
65 electrophoresis gel
66a a first lane in an electrophoresis gel
66b a second lane in an electrophoresis gel
66c a third lane in an electrophoresis gel
70 voltage source
71 electrical leads
72 cathode
73 anode
74 electrolyte
80 light source
81 excitation light
82 fluorescent light
83 light detector
90a capture membrane
90b a capture membrane for a first species
90c a capture membrane for a second species
90d a capture membrane for a third species
92 labeling
92a area with captured and labeled nucleic acids
92b area with a first captured and labeled biomolecular target
92c area with a second captured and labeled biomolecular target
92d area with a third captured and labeled biomolecular target
94 nucleic-acid binding surface
100 vertical transfer of nucleic acids into the manipulation platform
101 transfer of amplification products out of the manipulation platform
102 manipulation of nucleic acids
104 arrow representing transfer of molecules into the manipulation platform
105 arrow representing transfer of molecules out of the manipulation platform
110 positions of wells containing positive controls
111 positions of wells containing negative controls
112 positions of wells containing dye
120 features of a registration pattern imprinted onto a tissue sample
130 three wells showing CT values of 24
131 three wells showing CT values of 29.7
132 three wells showing CT values of 49.2
133 three wells showing negative control results
134 three wells showing CT values of 48.8
135 three wells with ROX background stain
140 large holes opening from the wells onto the top face of the substrate
141 small holes opening from the wells onto the bottom face of the substrate
150 frame of pressure apparatus
151 clamp of pressure apparatus
152 block of pressure apparatus
153 sealing film
155 thermocycler
156 scale

### IV. Terms

The following explanations of terms are provided to better describe the present disclosure and to guide those of ordinary skill in the art in the practice of the present disclosure. The singular forms “a,” “an,” and “the” refer to one or more than one, unless the context clearly dictates otherwise.

For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Adjuvant: An agent which is added to a mixture to improve its functionality, with generally minimal side effects. For instance, in the case of PCR amplification, detergents may be added to make target molecules more accessible to the DNA polymerase in the mix.

Amplifying a molecule: To increase the number of copies of a molecule, such as a nucleic acid molecule including a gene or fragment of a gene or a molecule of mRNA or small nuclear RNA or other RNA. The resulting products are called amplification products.

cDNA (complementary DNA): A piece of DNA lacking internal, non-coding segments (introns) and regulatory sequences which determine transcription. cDNA can be synthesized by reverse transcription from messenger RNA extracted from cells. It is necessary to create cDNA to amplify RNA, since RNA cannot be amplified directly.

Chamber: A vial, vessel, through-hole, tube, well, or small area (“patch”) of gel.

Contacting: Placement in direct physical association, such as placing a tissue section in direct physical association with the disclosed manipulation platform.

Crosstalk: The movement of target molecules or solid supports comprising target molecules from one chamber to one or more adjacent chambers.

DNA (deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

DNase: An enzyme that catalyzes the hydrolysis of DNA, thereby breaking it down or degrading it.

Differential expression: A difference, such as an increase or decrease, in the conversion of the information encoded in a gene into messenger RNA (mRNA), the conversion of mRNA to a protein, or both. In some examples, the difference is relative to a control or reference value, such as an amount of gene expression that is expected in a sample from a subject who does not have a disease. Detecting differential expression can include measuring a change in gene expression.

Expression: The process by which the coded information of a gene is converted into an operational, non-operational, or structural part of a cell, such as the synthesis of a protein.

Extraction: The process by which the biomolecules in a tissue sample are released from surrounding proteins, cells, and tissue-matrix so that they can diffuse freely into a solution surrounding the tissue.

Isolated: An “isolated” biological component (such as a nucleic acid molecule, protein, or cell) has been substantially separated or purified away from other biological components in which the component naturally appears.

Label: An agent capable of detection, for example by ELISA, spectrophotometry, flow cytometry, or microscopy. For example, a label can be attached to a nucleic acid molecule or protein, thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Lateral: The term “lateral” as used herein refers to the movement of nucleic acids parallel to the face of the 2D tissue sample or manipulation platform.

Layered expression scanning (LES) membrane: A thin sheet of material that can be treated to render it capable of capturing specific biomolecules, such as proteins or DNA sequences. For example, LES membranes can be treated to contain either antibodies or DNA sequences to capture either specific proteins or DNA sequences (as described in U.S. Pat. No. 6,602,661, which is hereby incorporated by reference in its entirety). The membranes can be stacked, with each membrane treated to capture a different target. The movement of biomolecules is primarily vertical (i.e., perpendicular to the faces of the membranes) through the stack of membranes through micron-scale track-etched vertical pores, so lateral diffusion is limited and micron-scale spatial resolution is maintained. Target fragment DNA or protein molecules are captured on the appropriate membrane, and the membranes are then separated and analyzed.

Lysis: The breakdown of cellular membrane, internal membrane, and any other internal or external cellular structural elements to enable the homogenization of individual cellular components or molecules. Generally, the membranes need to be broken down by surfactant and dense macromolecules which fill the space inside these membranes need to be disrupted by a protein denaturant.

Membrane: A thin sheet of natural or synthetic material that is porous or otherwise at least partially permeable to biomolecules.

Multiplexing: The simultaneous manipulation of multiple targets (e.g., sets of different targets, such as different genes) at the same time. This includes, for example, amplification carried out on more than one target molecule within the same chamber.

Nucleic acid: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded, circular or linear.

Nucleotide: A monomer that includes a base linked to a sugar, such as a pyrimidine, purine, or synthetic analogs thereof, or a base linked to an amino acid, as in a peptide nucleic acid (PNA). A nucleotide is one monomer in a polynucleotide, otherwise known as a nucleic acid. A sequence or nucleotide sequence refers to the sequence of bases in a polynucleotide.

Predetermined Characteristic: A distinguishing trait, quality, or property known to be associated with a certain condition, such as a disease, including acquiring a disease, severity of a disease, survival, and/or responsiveness to a certain treatment. Examples of a predetermined characteristic include a mutation in a gene, a methylation of a gene, or the expression level of a mRNA.

Polymerase chain reaction (PCR): An in vitro amplification technique in which a biological sample obtained from a subject (such as nucleic acids) is contacted with a pair of oligonucleotide primers, under conditions that allow for hybridization of the primers to a target nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) as well as other methods described throughout this disclosure.

A commonly used method for real-time quantitative polymerase chain reaction involves the use of a double stranded DNA dye (such as SYBR Green I dye). For example, as the amount of PCR product increases, more SYBR Green I dye binds to DNA, resulting in a steady increase in fluorescence. Another commonly used method is real-time quantitative TaqMan PCR (Applied Biosystems). This type of PCR has reduced the variability traditionally associated with quantitative PCR, thus allowing the routine and reliable quantification of PCR products to produce sensitive, accurate, and reproducible measurements of levels of gene expression. The 5′ nuclease assay provides a real-time method for detecting only specific amplification products. During amplification, annealing of the probe to its target sequence generates a substrate that is cleaved by the 5′ nuclease activity of Taq DNA polymerase when the enzyme extends from an upstream primer into the region of the probe. This dependence on polymerization ensures that cleavage of the probe occurs only if the target sequence is being amplified. The use of fluorogenic probes makes it possible to eliminate post-PCR processing for the analysis of probe degradation. The probe is an oligonucleotide with both a reporter fluorescent dye and a quencher dye attached. While the probe is intact, the proximity of the quencher greatly reduces the fluorescence emitted by the reporter dye by Förster resonance energy transfer (FRET) through space. Probe design and synthesis has been simplified by the finding that adequate quenching is observed for probes with the reporter at the 5′ end and the quencher at the 3′ end.

Primers: Short nucleic acid molecules, for instance DNA oligonucleotides 10-100 nucleotides in length, such as about 15, 20, 25, 30 or 50 nucleotides or more in length. Primers can be annealed to a complementary target DNA strand by nucleic acid hybridization to form a hybrid between the primer and the target DNA strand. Primer pairs can be used for amplification of a nucleic acid sequence, such as by PCR or other nucleic acid amplification methods known in the art.

Methods for preparing and using nucleic acid primers are described herein as well as, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.).

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. When referring to the purification of nucleic acids, this means isolating them from the rest of the components of the biological sample.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides. An oligonucleotide probe is a short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes.

RNA: A long chain polymer which is a complementary and modified form of the DNA in a cell. The term RNA generally implies the total RNA content of a cell, including messenger RNA (mRNA), ribosomal RNA, and transfer RNA, and is generally derived from the cytoplasm of a cell. RNA is distinct from DNA in that it is only single-stranded and contains a uracil base while DNA contains a thymine.

RNase (ribonuclease): A compound that catalyzes the hydrolysis of ribonucleic acid, thereby breaking it down or degrading it.

Sample: A material or matrix containing nucleic acids. In some examples, a sample contains biomolecules including tissue, gels, bodily fluids, and individual cells in suspensions or in pellets, as well as materials in containers of biomolecules, such as microtiter plates. A biological specimen or sample contains genomic DNA, RNA (including mRNA), protein, lipid, carbohydrate or combinations thereof, obtained from a subject. Examples include, but are not limited to, peripheral blood, urine, saliva, tissue biopsy, surgical specimen, amniocentesis samples, and autopsy material. In one example, a biological sample includes a tissue biopsy.

Target molecule: A molecule of interest. In the context of PCR, the target is the gene or other sequence that is amplified.

Total RNA: A term used herein to indicate primarily the cytoplasmic mRNA, but also small nuclear, small interfering, ribosomal, transfer, and any other kind of RNA which can be distinguished by its base pair sequence.

Two dimensional (2D) spatial information: A sample with 2D spatial information can have a non-uniform distribution of those nucleic acids. This includes, but is not limited to, a tissue section, a tissue section, an array of core samples from a specimen, an array of tissue-containing needles, an arrangement of cells adhering to a backing, a cell culture, a block of tissue, a tissue section encased in a gel or other matrix, nucleic acids contained within a gel or other matrix, a biopsy, or an organ.

Sample matrix: The material in a sample that contains the nucleic acids. This includes, but is not limited to, biological tissue and gels.

Surface coating: A term used herein to describe a material and process for making a material where a first substance or substrate surface is at least partially covered or associated with a second substance. Further, when a device is “coated” as used herein, the coating may be effectuated by any chemical or mechanical bond or force, including linking agents. Thus a device composed of a first substance may be “coated” with a second substance via a linking agent that is a third substance. As used herein, the “coating” need not be complete or cover the entire surface of the first substance to be “coated”. The “coating” may be complete as well (e.g., approximately covering the entire first substance). There can be multiple coatings and multiple substances within each coating. The coating may vary in thickness or the coating thickness may be substantially uniform. In one example, a chamber is coated with a surface coating, such as a hydrophilic substance (e.g., BSA).

Target molecule: A molecule, or a portion of a molecule, of interest. For example, in the context of PCR, the target may be a gene or other sequence that is amplified.

Under conditions sufficient for: A phrase that is used to describe any environment that permits the desired activity. In one example, this includes performing the polymerase chain reaction for a time period sufficient to permit the manipulation of one or more preselected nucleic acid molecules, such as the manipulation of one or more nucleic acid molecules that are diagnostic of a disease state. In other examples, the phrase includes treating the sample to free nucleic acids from the sample matrix.

Vertical: The term “vertical” as used herein refers to the movement of nucleic acids perpendicular to the face of the sample or the manipulation platform.

### V. Methods for Loading Target Nucleic Acids

Disclosed herein are methods of loading target nucleic acids into a manipulation platform (also referred to as a substrate) having a plurality of chambers while maintaining the 2D spatial relationship between the nucleic acids that were present in the original sample having 2D spatial information. In one example, the method of loading includes providing the sample to the substrate including the plurality of chambers; transferring portions of the sample into the plurality of chambers; and providing conditions sufficient to free nucleic acids from the transferred biological sample portions within the plurality of chambers.

In a disclosed method of loading, the nucleic acids are placed into an aqueous environment within the chambers that allows subsequent manipulation or detection of nucleic acids while maintaining the 2D spatial relationship of the nucleic acids relative to those in the original biological sample throughout the method in the substrate. Exemplary samples having 2D spatial information include a tissue section, an array of core samples from a specimen, an array of tissue-containing needles, an arrangement of cells adhering to a backing, a cell culture, a block of tissue, a tissue section encased in a gel or other matrix, nucleic acids contained within a gel or other matrix, a biopsy, or an organ. In one example, providing the sample includes contacting the sample to a first surface of the substrate. In some examples, providing conditions sufficient to free nucleic acids include a digestion of proteins (such as with proteinase K or trypsin), an inactivation of the digestion, a denaturation of nucleic acids or proteins, a purification of free nucleic acids or proteins, or a combination of two or more thereof.

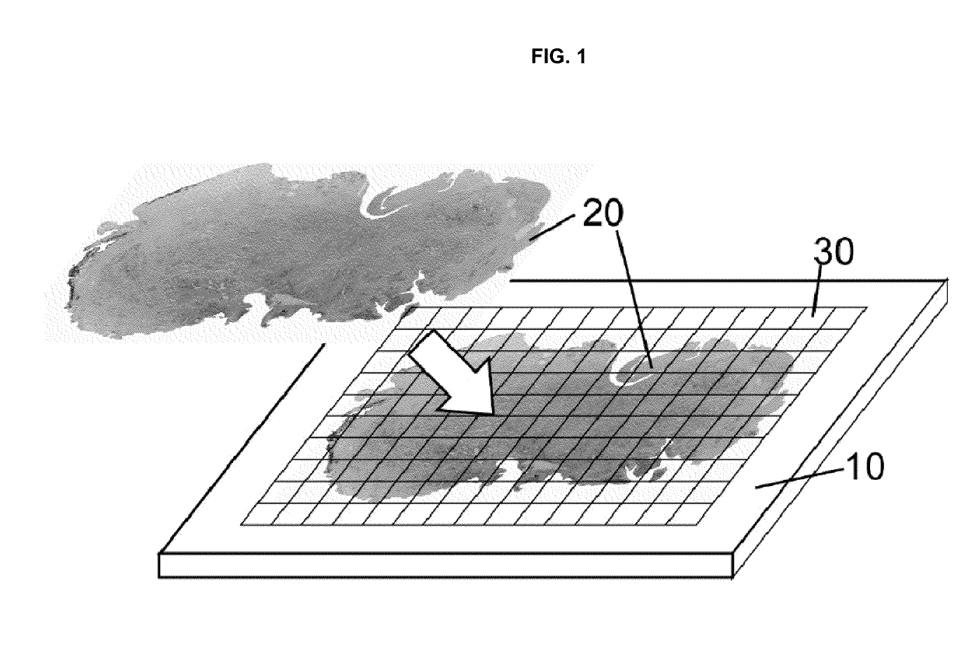

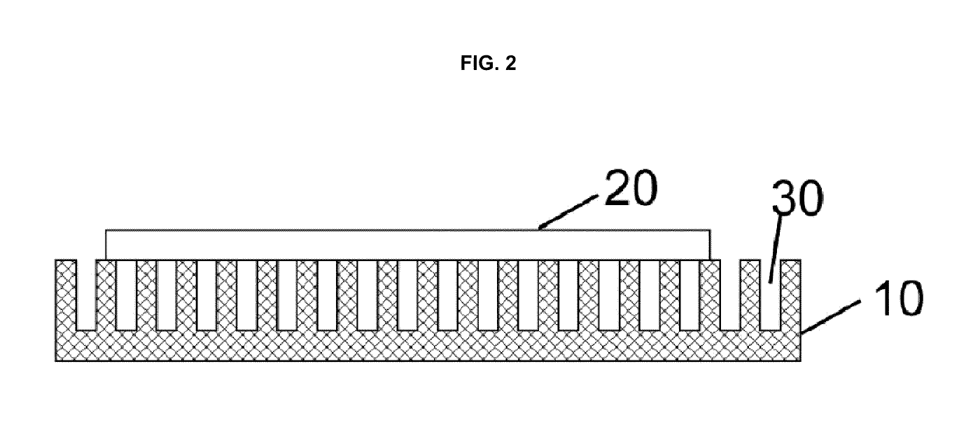

FIGS. 1-4B illustrate an embodiment of the disclosed method for loading target nucleic acids from a sample having 2D spatial information into a device, such as a manipulation and detection platform having chambers, hereinafter referred to as a manipulation platform with the understanding that detection of nucleic acids within the platform may also be performed. FIG. 1 shows a perspective view of sample 20 before and after it is positioned on an outer surface of manipulation platform 10 having microscale chambers or wells or vials 30. The arrow schematically indicates the placement of sample 20 onto manipulation platform 10. In this example, sample 20 is provided to manipulation platform 10 so that the 2D spatial relationship of nucleic acids within biological sample 20 is maintained (e.g., the tissue is positioned parallel to the outer surface of manipulation platform. FIG. 2 illustrates a cross-sectional view of sample having 2D spatial information 20 after it is placed on the outer surface of manipulation platform 10. In manipulation platform 20 of FIG. 2, chambers 30 are wells or vials.

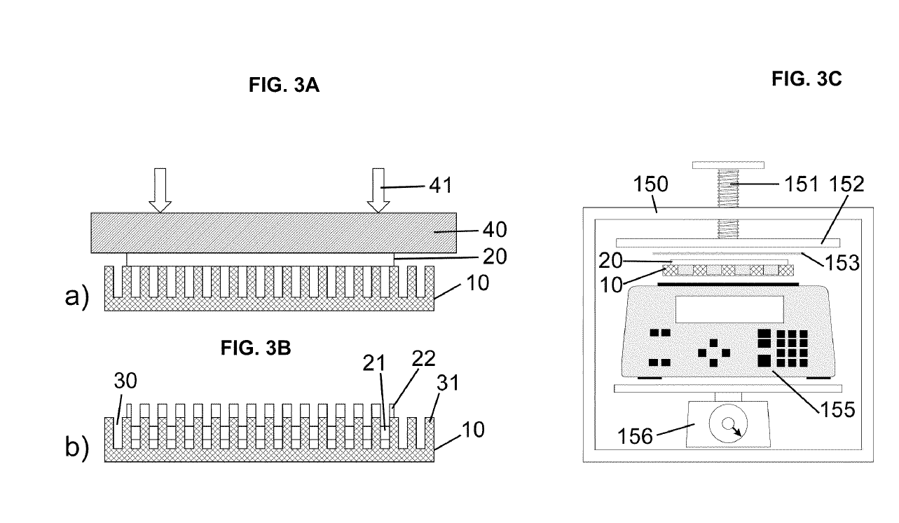

FIGS. 3A and 3B show cross-sectional views of the method of transferring the nucleic acids from sample having 2D spatial information 20 into chambers 30 using pressure 41 to physically push portions of sample 20 into wells/vials 30. FIG. 3A shows plate 40 placed on top of sample 20, and pressure, indicated schematically by arrows 41, being applied to plate 40. FIG. 3B shows the result of this application of pressure in which biological sample 20 that was originally positioned over each micro-scale well 21 has been pushed into the micro-scale well 30, separating it into tissue portions. Portions of sample 22 that originally overlaid walls 31 that separate wells 30 from each other remain over walls 31 and do not go into chambers 31. This method of transferring nucleic acids 23 in sample 20 into micro-scale wells 30 preserves the original 2D spatial relationship of nucleic acids 23 relative to the original sample because biological sample 21 is pushed straight down without distorting the 2D spatial relationship of nucleic acids 23.

In an example, the resolution of manipulation platform 10 is the lateral distance between the beginning of one chamber and the next, or the size of the well plus the width of the wall between wells. By making the wells small and the walls thin, the resolution of manipulation platform 10 is increased, allowing the molecular content of biological sample 20 to be mapped down to smaller scales, such as down to the level of several cells or even sub-cellular levels. For example, if the wells are 60 μm on each side and if the biological cells have a diameter of 20 μm, then molecular information from between 3*3=9 and 4×4=16 cells will be combined within each well, depending on the registration of the cells over the well. If the walls are 20 μm in thickness, then the resolution of the manipulation platform will be 80 μm. The fill factor, or fraction of the substrate that can contain molecular information, is the ratio of the well area over the total area. For example, if the wells are 60 μm on each side and the walls are 20 μm in thickness, then the fill factor is (60*60)/((60+20)*(60+20))=(60*60)/(80*80)=3600/6400=0.56.

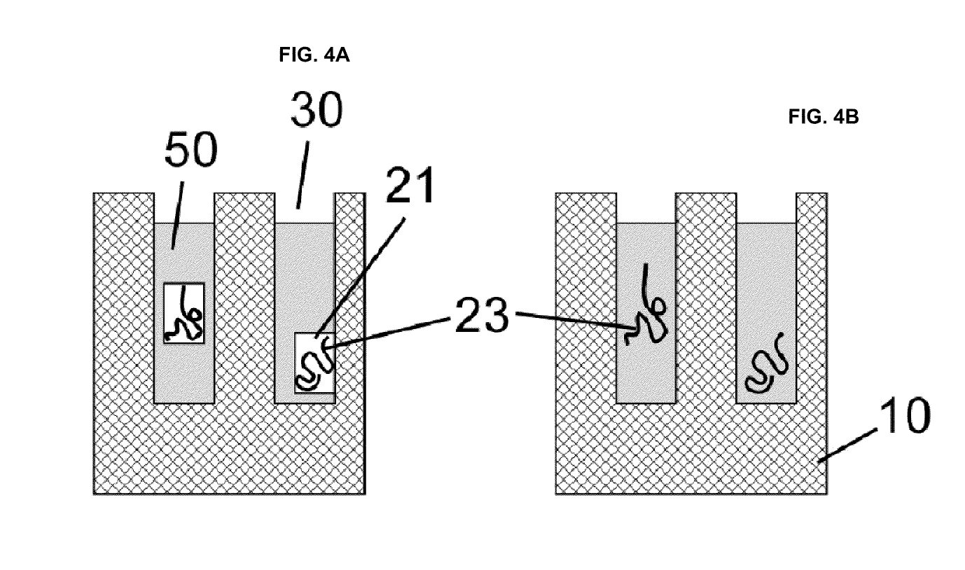

FIG. 3C illustrates an apparatus for applying pressure to sample having 2D spatial information 20 to transfer and subsequently amplify nucleic acids in plurality of chambers of manipulation platform 10. In an embodiment, the apparatus includes frame 150 for supporting clamp 151. In one example, clamp 151 is secured to frame 150 by a fastener (e.g., a screw) which allows the clamp to be manipulated, such as raising or lowering the clamp by rotation. Block 152 is positioned adjacent to clamp 151 so that, when clamp 151 is lowered, block 152 comes into contact with sealing film 153 which covers sample 20, which in turn contacts manipulation platform 10. Coupled to manipulation platform 10 is thermocycler 155, which in turn is coupled to scale 156. The scale may be omitted, and force can be externally calculated based on a torque wrench, or calculated by number of turns applied for compression. The thermocycler may include a detection device, such as a fluorescence reader, that allows real-time detection of amplification products. In one example, block 152 is lowered until the desired pressure is reached (for example, as indicated by scale 156). In one example, the pressure is kept applied during thermocycling. FIGS. 4A and 4B illustrate freeing nucleic acids 23 from biological sample 21 inside vials/wells 30. FIG. 4A shows digesting reagent 50 added to micro-scale vials/wells 30. Reagent 50 degrades biological sample 21, but not nucleic acids 23 of interest (e.g., such as RNA, DNA, or protein). As shown in FIG. 4B, this frees nucleic acids 23 for subsequent manipulation or detection in same substrate 10. This method of freeing nucleic acids 23 preserves their 2D spatial relationship because nucleic acids 23 are contained within micro-scale wells 30 and cannot move horizontally.

After performing the steps shown in FIGS. 2-4B, nucleic acids 23 have been transferred from sample 20 into manipulation platform 10, where they can be subsequently manipulated and/or detected, and nucleic acids 23 have the same relative spatial location as they did in the original sample prior to transferring.

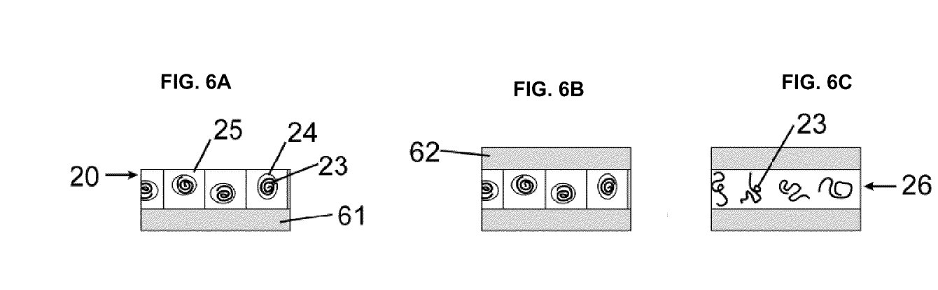

Additional exemplary methods of loading target nucleic acids from a sample having 2D spatial information into a device, such as a manipulation and detection platform having chambers are shown in FIGS. 6A-9. Also, the steps can be carried out in a different order, and the manipulation platform can have chambers of different types as described in detail herein or those known to one of ordinary skill in the art.

FIGS. 6A-6C shows an additional method for freeing nucleic acids 23 from sample 20. As illustrated in FIG. 6A, sample 20 is positioned adjacent to (such as on top of) layer of gel 61. Nucleic acids 23 in this case are nucleic acids within nuclei 24 of cells 25. Protein-digesting reagents are then added to the tissue. As illustrated in FIG. 6B, second layer of gel 62 is placed on top of sample 20 (thereby encasing the components of the digested sample in a gel). This gel encasement prevents the components of the digested tissue from moving laterally, thereby preserving the 2D spatial positions of the nucleic acids. The digested tissue encased in gel with the nucleic acids freed for subsequent manipulation or detection is illustrated in FIG. 6C.

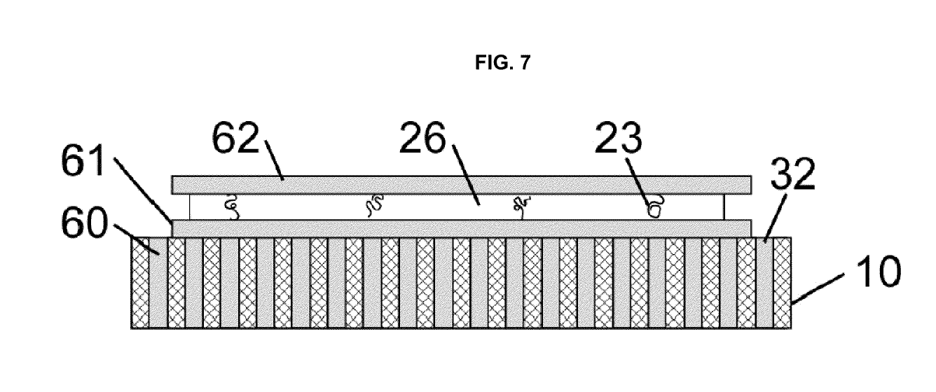

FIG. 7 shows another example of the method of providing digested 2D tissue sample 26 to manipulation platform 10 with chambers 30. In this example, the chambers are micro-scale through-holes 32 which are filled with gel 60. The sandwich of nucleic acids 23 encased in gel 61, 62 prepared as illustrated in FIGS. 6A-6C is brought into contact with manipulation platform 10. The 2D positions of the nucleic acids are maintained because they are entrapped in gel.

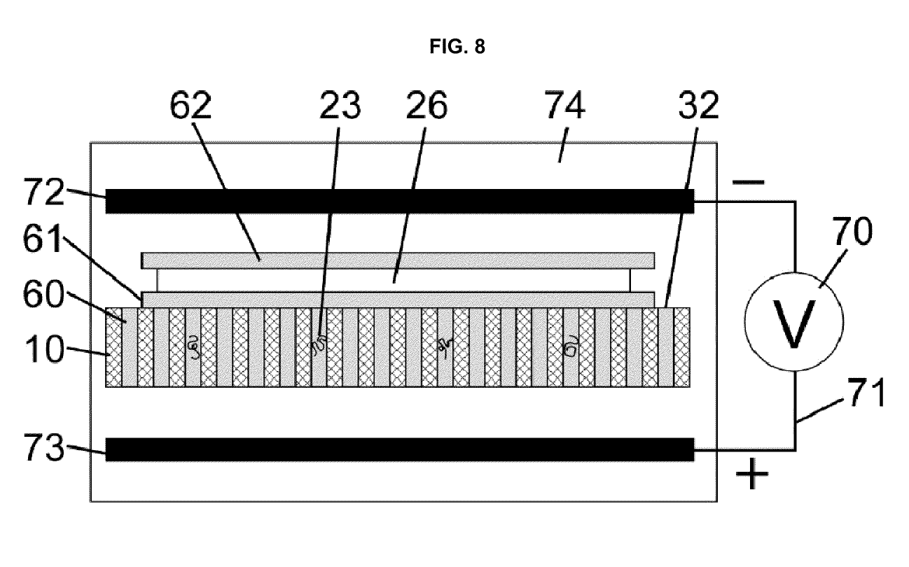

FIG. 8 shows another method of transferring nucleic acids 23 from sample 20 into manipulation platform 10 using electrophoresis rather than pressure. With this method, nucleic acids 23 are charged, and in the figure nucleic acids 23 are negatively charged nucleic acids, such as nucleic acids. Manipulation platform 10 and the tissue/gel sandwich of FIG. 7 are placed between electrodes 72, 73 inside appropriate electrolyte 74. One electrode is anode 73 and the other is cathode 72. Voltage source or power supply 70 with leads 71 to electrodes 72, 73 creates an electric field. The negatively charged molecules move out of digested sample matrix 26 toward anode 73, through gel 61 and into gel 60. Uncharged and positively charged molecules do not move into manipulation platform 10. This partially purifies sample 20 in addition to transferring nucleic acids 23 into micro-scale chambers 32. The 2D spatial relationship of the transferred nucleic acids relative to the original sample is retained because they move vertically in the field, without significant lateral motion. In some examples, for highest resolution, there is no or minimal lateral motion of species into adjacent vials. For example, the lateral motion is less than 50% of the width of the walls separating the chambers.

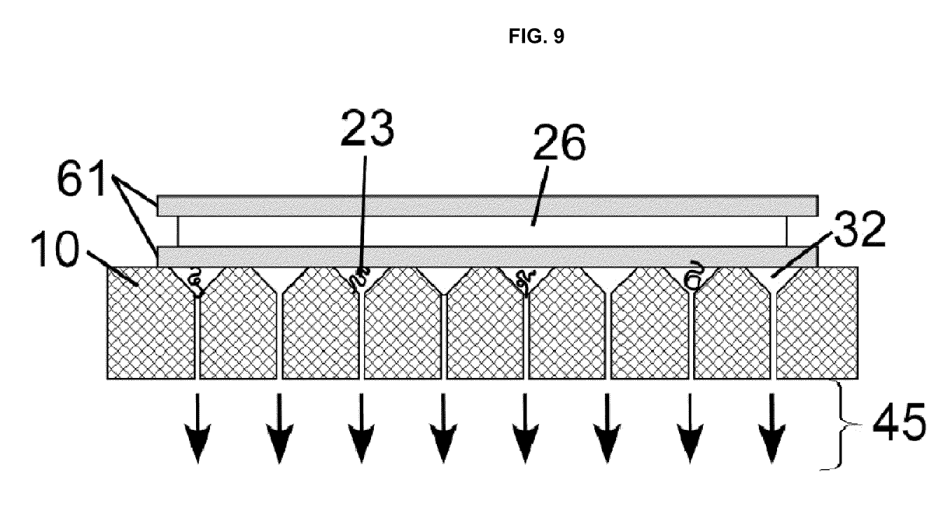

FIG. 9 shows an additional example of a method of transferring target nucleic acids 23 into manipulation platform 10 with through-hole style micro-chambers 32. Suction force 45, represented by arrows, pulls previously freed nucleic acids 23 into through-hole micro-scale chambers 32. Since the force is perpendicular to the plane of sample 20, the relative spatial positions of nucleic acids 23 are maintained. In the embodiment of FIG. 9, micro-scale through-hole chambers 32 have a larger opening at the top than at the bottom. Such holes could be formed by any methods known to those of ordinary skill in the art, including a combination of anisotropic wet chemical etching and deep reactive ion etching.

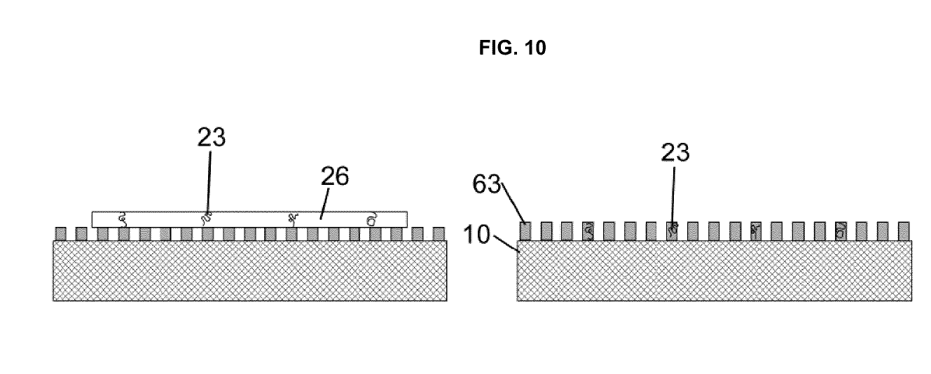

FIG. 10 provides an even further example of transferring target nucleic acids 23 into manipulation platform 10. In this Figure, chambers 30 are small areas of gel patterned onto the surface of a substrate. Exemplary substrates include glass, quartz, silicon, metal, polycarbonate, and other polymers; the substrate can have the form of a plate, a fiber, a cantilever beam, or other shape. In FIG. 10, digested sample matrix 26 is brought into contact with the patterned gel. Nucleic acids 23 are freed from digested sample matrix 26 either before or after this step. Some of target nucleic acids 23 diffuse from biological sample 20 into gel areas 63. Nucleic acids 23 are then trapped in gel 63 after digested sample matrix 26 is removed (as illustrated in the schematic on the right). The 2D spatial relationships are maintained because the vertical diffusion distance into the gel is minimal compared to the lateral distance between different patches of gel and because nucleic acids 23 within one patch of gel cannot readily traverse the areas without gel to get to an adjacent patch. For example, a small vertical distance allows only minimal lateral diffusion in the time that the species are transferred by vertical diffusion. For highest resolution, lateral motion should be less than 50% of the distance between the gel patches. Heat and fluid may optionally be added during diffusion to facilitate this process.

### VI. Manipulation Platforms with Chambers

Manipulation platforms 10 with chambers 30 are disclosed herein. These platforms can be utilized to perform the disclosed methods or be included within the disclosed systems.

In an embodiment, manipulation platform 10 includes a substrate having a plurality of wells, wherein each well includes a body with at least a first opening on a first face, or top face, of the substrate and an inner surface of the body. The material separating the interiors of the plurality of wells, and thereby defining the inner surfaces of the wells, makes up the walls of the well. In another embodiment, the body of the well also has a second opening onto the second face of the substrate, forming a through-hole style micro-scale chamber 32 or mm-scale chamber 33.

Methods for forming wells and chambers in substrates are well known to those skilled in the art. They include photolithography followed by wet chemical etching, electro-discharge machining, dry chemical etching, reactive ion etching, and deep reactive ion etching. These methods are described in standard textbooks (such as G. T. A. Kovacs, Micromachined Transducers Sourcebook, (WCB McGraw-Hill, Boston, 1998), pp. 57), and instruments for carrying out the methods disclosed herein are commercially available and are standard equipment in microfabrication laboratories. Methods for forming manipulation platforms 10 also include molding or stamping processes, such as hot embossing. The latter involves pressing a polymeric material that softens upon heating (a thermoplastic), such as polycarbonate, against a mold at an elevated temperature. The polymer is shaped by the mold. This method allows rapid and inexpensive manufacture of the platforms.

Manipulation platforms 10 with through-hole style micro-chambers 32 can be sealed at the bottom end to create well-style micro-chambers. This can be done by the application of a sealing layer or material on the bottom face of the substrate, such as a sealing tape or mineral oil. This seal can be temporary, since it is reversible upon removal of the sealing layer or material. Furthermore, to aid in reducing or preventing evaporation and crosstalk during manipulation of nucleic acids 23, chambers 30 of manipulation platform 10 can be sealed also on the top end to create chambers without openings by applying a sealing material to the top surface of the substrate. This can be done after transferring nucleic acids 23 into plurality of chambers 30 and prior to manipulating nucleic acids 23 within plurality of chambers or wells 30. The sealing composition can be added and removed at various stages of the manipulation protocol, such as prior to the addition of reagents, prior to heating, or prior to transferring the manipulated molecules out of the wells for detection.

In some examples, manipulation platform 10 includes chambers having a diameter or edge length of 1-2000 micrometers, such as about 100-1700 micrometers (μm). In a particular example, the diameter is about 5 to 300 μm, such as 20 to about 150 μm, 40 to 110 μm, 50 to 80 μm, including 10 μm, 20 μm, 30 μm, 40 μm, 50 μm, 60 μm, 70 μm 80 μm, 90 μm, 100 μm, 110 μm, 120 μm, 130 μm, 140 μm, 150 μm, 160 μm, 170 μm 180 μm, 190 μm, 200 μm. In some examples, the chambers are separated by a distance of 1-500 micrometers, such as about 5 to 400 μm, such as 10 to about 200 μm, 30 to about 150 μm, 40 to 110 μm, 50 to 80 μm, including 10 μm, 20 μm, 30 μm, 40 μm, 50 μm, 60 μm, 70 μm 80 μm, 90 μm, 100 μm, 110 μm, 120 μm, 130 μm, 140 μm, 150 μm, 160 μm, 170 μm 180 μm, 190 μm, 200 μm. Exemplary manipulation platforms 10 can be formed of various compositions known to those of ordinary skill in the art, including silicon and aluminum. In one example, the surface of the silicon-based manipulation platform 10 is coated with silicon dioxide or silicon nitride. The manipulation platforms can also be formed of glass, quartz, or polycarbonate, or of materials on top of silicon, glass, or other substrates, including polymers such as SU8 and parylene C.

i. Surface Coatings for Manipulation Platforms

Adsorption of reactants on the walls of micro-scale reaction chambers can quench the reaction in some cases without appropriate surface coatings. For example, the high surface-to-volume ratio in micro-wells can result in non-specific adsorption of Taq DNA polymerase and template DNA on the well walls. In one example, the walls of the chambers are treated with a hydrophilic substance to reduce this adherence. This treatment can occur at various stages during the method, including prior to providing sample 20 to manipulation platform 10, or prior to the addition of reagents to perform the amplification of the target nucleic acids. This treatment ensures that the micro-chamber side-walls are sufficiently hydrophilic to allow aqueous solutions to fill the chambers.

In some examples, the surfaces are silanized by covalently bonding an R group onto the Si—O—H moieties (such as, silanization with CH₃(CH₂)₂SiCl₃ or (CH₃)₂SiCl₂). In certain examples, wells are treated with a hydrophilic substance, such as a surface coating which enables or enhances PCR in difficult environments by rendering surfaces of the environment more favorable for enzymatic reactions. Typically these are highly biocompatible polymeric water-soluble substances, with a varying range of molecular weights. Illustrative examples include bovine serum albumin, a combination of silicon dioxide coating with bovine serum albumin, silianization, surface coating with polyacrylamide, coating with parylene, Triton X-100, Tween-20, poly ethylene glycol, polyvinyl pyrolyidine, and polysucrose. Because these additives are known to affect PCR to varying degrees, they are considered interchangeable, specifically that they can be switched or combined when a particular formula is non-optimal. Further chemicals can be included in this group including those commercially available (see, world wide web address sigmaaldrich.com/etc/medialib/docs/Aldrich/Bulletin/al_ms_app_catalog_biomat.pdf) and known to those of ordinary skill in the art (see, U.S. Pat. Nos. 6,127,188, 6,716,629, Lou et al. 2004 Biotechniques 36:248, Kricka and Wilding 2003 Anal Bioanal Chem 377:820, each of which is incorporated herein by reference in its entirety).

In a particular example, a solution of 1:20:20 BSA:water:ethanol is used to treat Al or Si micro-well surfaces prior to loading with PCR reagents. The solution is applied to the surface of the micro-well array and then allowed to dry, coating the walls of the wells. The resulting surface coating is stable at room temperature and upon rinsing in water, ethanol, or acetone. The BSA coating on the surfaces of manipulation platform 10 renders the surfaces hydrophilic so that reagents can be loaded into chambers 30, and the coating also minimizes interactions of nucleic acids 23 within chambers 30 with the sidewalls, enhancing PCR. FIG. 8 shows a cross-sectional view of manipulation substrate 10 for placing target nucleic acids 23 into an environment that allows preservation, manipulation, and/or detection while preserving the 2D spatial positions of target nucleic acids 23 as they were originally in sample. Two-dimensional biological D sample including nucleic acids 23 encased within gel layers 61, 62 is placed on the surface of manipulation platform 10. Manipulation platform 10 has chambers 30 that are of the form of through-holes 32 filled with a gel 60. Manipulation platform 10 and sample 20 are placed between anode 73 and cathode 72 connected by leads 71 to power supply 70. Electrolyte 74 is also supplied between anode 73 and cathode 72. An applied voltage between anode 73 and cathode 72 provides an electrophoretic means for transferring target nucleic acids 23 from sample 20 into chambers 30, allowing nucleic acids 23 to be held, manipulated, or detected. In the embodiment of FIG. 8, manipulation platform 10 includes gel 60 within chambers 30. Gel 60 can be reversibly dried and hydrated. One of the purposes of the gel is preventing or reducing evaporation and crosstalk. The step of drying the gel permits the addition of additional reagents, dyes, and other biochemical species to the chambers, while those nucleic acids and chemical species that are already in micro-chamber wells 30 are retained, and therefore held in their original 2D position, by gel 60.

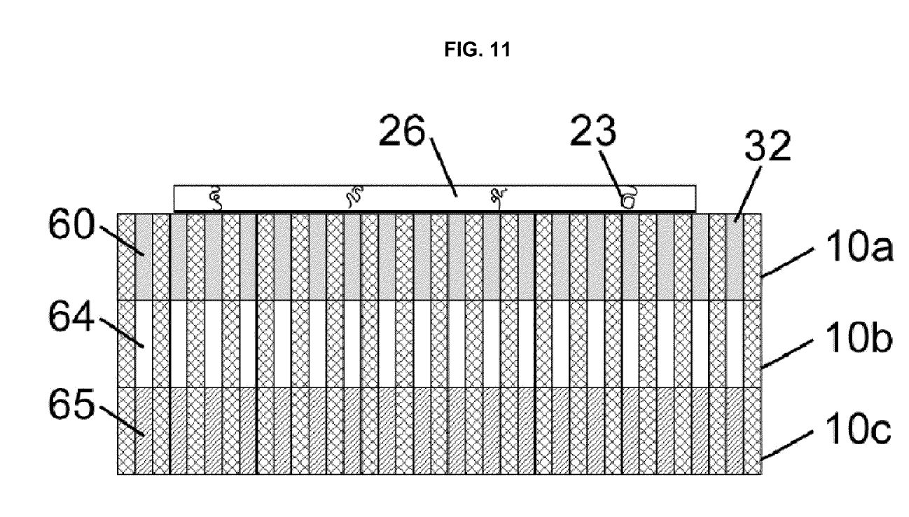

FIG. 11 shows manipulation platform 10 comprising three substrates 10a, 10b, and 10c with through-hole style micro-scale chambers 32 that have been aligned or registered. A first step of sample manipulation can take place in platform 10a, for example filtration or binding, followed by transfer to substrate 10b, where another manipulation can take place, such as PCR, followed by transfer to substrate 10c, where a third manipulation step and/or detection can take place, such as fluorescent tagging. The interior of the wells of the different substrates may contain different materials, such as gel 60, to aid the different manipulation and detection procedures.

ii. Additional Manipulations Within the Chambers

Among further manipulations that can be performed within chambers 30 are the following: (1) digestion to break proteins into fragments, allowing nucleic acids to become accessible (such as with proteinase K or trypsin); (2) inactivation of previously added reagents (such as inactivation by heat of proteinase K); (3) treatment of the tissue to inactivate RNases so that RNA molecules are preserved from degradation and available for subsequent manipulation and/or detection; (4) PCR, rolling circle amplification, or loop-mediated amplification, helicase-dependent amplification, or ligation chain reaction to amplify target nucleic acid sequences; (5) bisulphite modification to tag methylation of a gene; staining; rinsing the contents of wells; binding proteins to beads with bound antibodies; binding nucleic acids to beads with bound oligonucleotides; application of electromagnetic radiation for detection, heating; desiccating or dehydrating the contents of wells; or a combination of two or more thereof. By selective amplification of target nucleic acids, such as nucleotide sequences, several characteristics of nucleic acids can be mapped, including a mutation in a gene, a methylation of a gene, or the expression level of an mRNA.

### VII. Methods and Systems for Analyzing Nucleic Acids

Disclosed herein are methods and systems for analyzing nucleic acids. In one embodiment, methods, and systems for analyzing nucleic acids, such as nucleic acids (i.e., DNA, RNA) which have been transferred from a tissue section into a disclosed manipulation platform 10 for the purpose of rapidly mapping nucleic acid patterns at high resolution in tissue samples, such as for identifying the molecular micro-environments of physiological and pathophysiological samples are provided. Methods and systems for analyzing nucleic acids are also disclosed which employ the disclosed manipulation platform 10 to map gene and protein expression as well as genetic alterations. For example, methods of using manipulation platform 10 disclosed herein for rapid mapping of gene expression and genetic alterations as a function of spatial position in a sample, such as for identifying the molecular micro-environment of physiological and pathophysiological samples, are also disclosed.

In an embodiment, the method includes analyzing nucleic acids which have been transferred from sample 20 into manipulation platform 10 with chambers 30 while retaining the spatial locations of nucleic acids 23. Exemplary samples having 2D spatial information include a tissue section, an array of core samples from a specimen, an array of tissue-containing needles, an arrangement of cells adhering to a backing, a cell culture, a block of tissue, a tissue section encased in a gel or other matrix, nucleic acids contained within a gel or other matrix, a biopsy, or an organ. Any means known to one of skill in the art can be used to transfer nucleic acids 23 into manipulation platform 10 prior to analyzing nucleic acids 23 including those described in detail herein (such as, electrophoresis, pressure, and/or suction).

In some embodiments of the method, the method further includes treating substrate 10 with agarose prior to providing sample 20 to substrate 10. In certain embodiments, the method includes applying a sealing material to the second face of substrate 10 prior to providing sample 20 to substrate 10.

In some embodiments, the method for analyzing nucleic acids includes manipulating nucleic acids 23 which can include purifying the nucleic acids as well as amplifying the target nucleic acids by methods known to those of skill in the art (such as PCR, rolling circle amplification or loop-mediated amplification) as well as described in detail herein. In a particular example, target nucleic acids are amplified by PCR in the presence of blocking agent (such as bovine serum albumin) and amplification reagents, wherein the blocking agent is added prior to the amplification reagents and comprises about 0.1% to 1% of the total volume of an amplification reaction, thereby amplifying target nucleic acids while preserving the 2D spatial relationship of target nucleic acids relative to their original position in the original sample throughout the method in substrate 10.

The disclosed methods for analyzing nucleic acids further include detecting such nucleic acids including by fluorescent labels, radioisotope labeling, and dyes.

In some particular embodiments, the biological sample is obtained from a subject either predisposed to developing cancer or is known to have cancer. In a particular example, the sample is a prostate tissue sample. For example, this technology can be used to study tumorigenesis, such as prostate tumorigenesis, providing high resolution multi-dimensional maps (i.e., maps of more than one target nucleic acid) of gene expression in samples, such as tissue sections. For example, the disclosed methods can be used for high-resolution DNA or mRNA mapping, such as methylated DNA and GSTP1 mRNA mapping. These measurements can provide information on the molecular basis of disease for cancer researchers, and they can be either used alone or in combination with existing technologies, such as immunohistochemistry techniques.

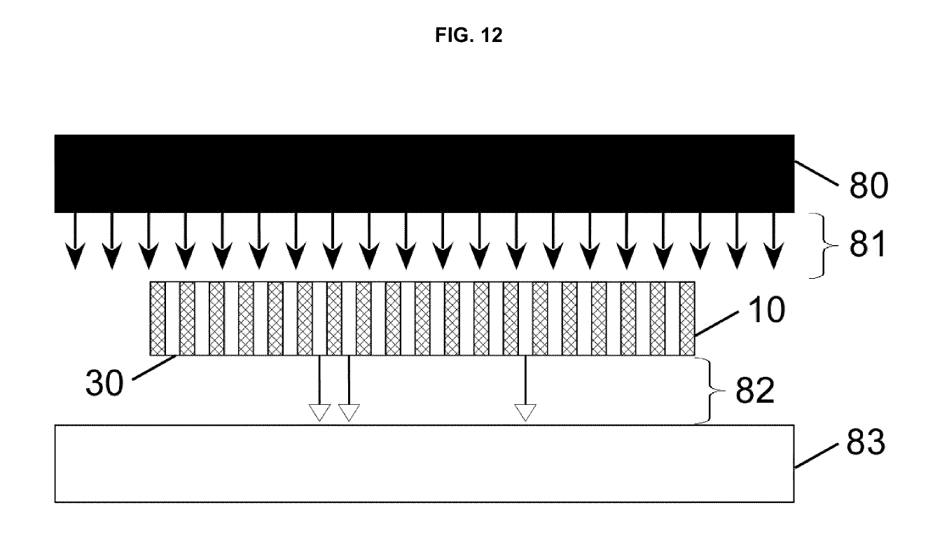

In an embodiment, methods of analyzing nucleic acids include transferring nucleic acids 23, such as DNA and mRNA, from a sample, such as a tissue section, onto an underlying manipulation platform 10 while retaining nucleic acids' 23 spatial locations (as illustrated in FIGS. 12-14). In one particular embodiment, nucleic acids can be amplified within the optimal environment of chambers 30, and then transferred out of chambers 30 onto nucleic acid-binding membrane 94 for visualization. The end result is a spatial map of epigenetic changes and gene expression throughout the tissue: in the tumor focus, at the edge of the tumor, of the surrounding possibly abnormal tissue, and in normal tissue. Such maps can be produced rapidly and at minimal cost, making it feasible to map several locations in the tissue or organ, yielding 3-dimensional maps.

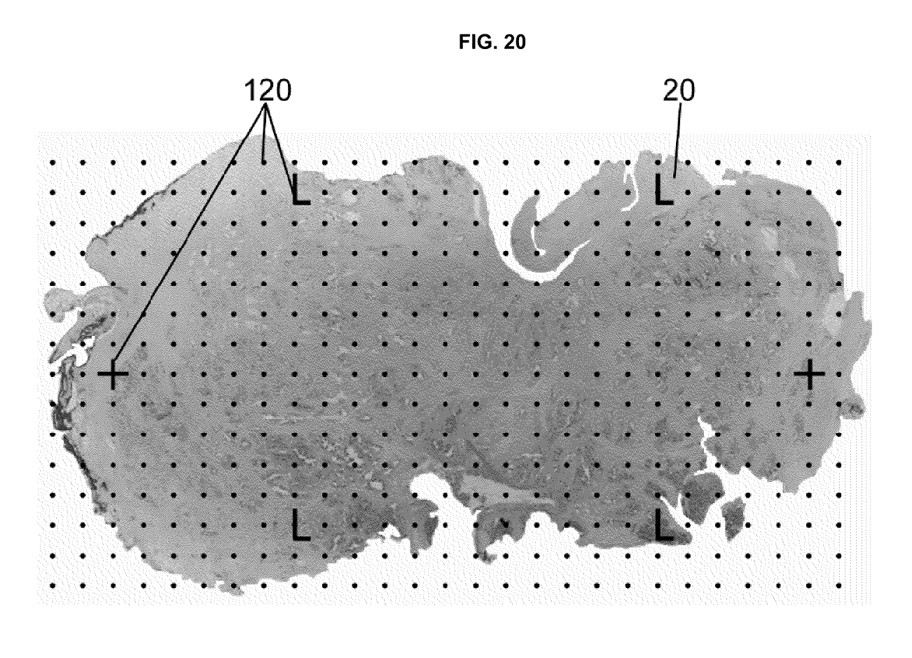

In some particular examples, methods of analyzing nucleic acids further include placing registration marks 120 on biological sample 20, such as shown schematically in FIG. 20, to allow the molecular maps and the histology to be overlaid and compared, for example in a computer, thereby facilitating the analysis of biological sample 20. For example, larger marks provide for coarse alignment, and handed marks (such as L-shapes) provide for correct orientation. A grid pattern allows for detection and correction of tears, wrinkles, and other tissue imperfections. In some examples, alignment marks are placed onto or into biological sample 20 prior to sectioning. Marks can include, but are not limited to, physical holes or a particular molecular species that can be separately detected.

In an embodiment, a system for analyzing nucleic acids includes a transferring means, such as pressure, suction, electrophoresis or other transferring means known to those of ordinary skill in the art coupled to a manipulation platform which in turn is coupled to an amplification device, such as a thermocycler, coupled to a detection device, such as spectrometer. FIG. 3C provides an exemplary system for analyzing nucleic acids utilizing the methods and devices disclosed herein (as described in detail in Section V).

The methods and systems for analyzing nucleic acids allow the 2D spatial orientation pattern of the molecules with regard to the original sample to be maintained throughout the method.

### VIII. Methods for Purifying Nucleic Acids

Studies of mRNA in biological tissue are extensively performed in human clinical testing as well as the research fields of functional genomics, epigenetics, and biomarker discovery. This is because the mRNA needs to be released from the samples with high efficiency, such that nearly all of the mRNA is removed from the cells without degradation by native tissue ribonucleases (RNases), which break down RNA. Furthermore, the extraction and purification method must produce mRNA that is sufficiently free of tissue lysates and other species that interfere with downstream applications. In particular, any RNase left in the purified sample will quickly break down the RNA and impede downstream applications. Some common downstream applications include microarray studies, quantitative real-time PCR, quantitation by spectroscopic methods and qualification.

Nucleic acid isolations begin with cells extracted from tissue or cells grown in culture or suspension, called a tissue sample. The vast majority of techniques require the user to mechanically homogenize the tissue sample in a first vessel, typically a PCR or centrifuge tube. This mechanical lysis may include re-pipetting, grinding the tissue at liquid nitrogen temperatures, or grinding the tissue using glass beads and a shaker. Next the tissue is dissolved and further homogenized in an aggressive lysis solution, and then any remaining solids are separated out. Subsequent steps in this class of approaches then require at least a second vessel for purification and detection, but they most often specify a second vessel for purification, a third vessel for elution, and a fourth vessel for detection.

Disclosed herein is a method for isolating nucleic acids from a biological sample, such as a tissue sample, more rapidly and directly than is known in the art by performing the steps of lysis, homogenization, and purification in a single container (or vessel). The disclosed method also enables subsequent detection of the nucleic acids directly in the vessel. This method uses substantially fewer vessels and pipetting steps than the state of the art, can be performed in less time, and can be amenable to high-throughput applications and automation.

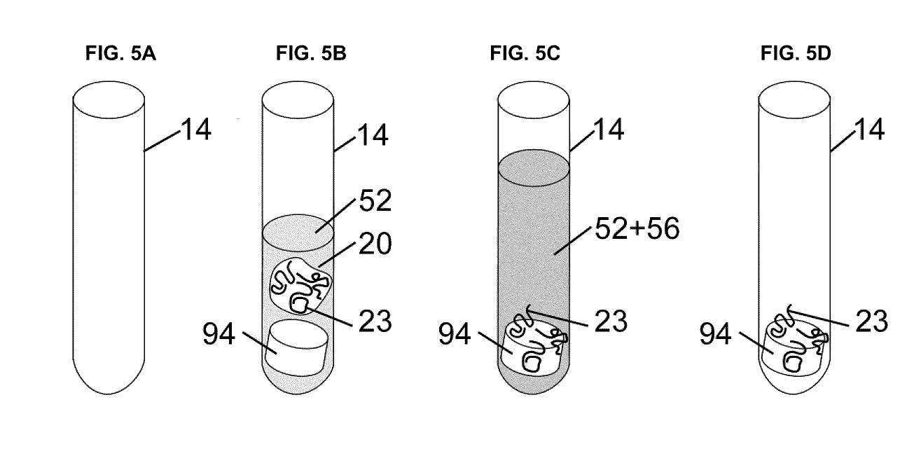

FIGS. 5A-5D show an exemplary method for purifying RNA in a single vessel. Biological sample 20, a nucleic acid binding surface 94, and reagents for denaturing protein are added to vessel 14 and allowed to react. Nucleic acid precipitation agent 56 is added, and then the unbound species are removed, leaving purified nucleic acids 27 on nucleic-acid binding surface 94. In particular, the method includes providing vessel 14 (FIG. 5A) and providing nucleic-acid binding surface 94, biological sample 20 containing nucleic acids 27, and protein denaturing agent 52 to vessel 14. As a result, nucleotides 27 are released from biological sample 20 (FIG. 5B). The method also includes adding nucleic acid precipitation agent 56 (FIG. 5C). As a result, nucleic acids 27 bind to nucleic-acid binding surface 94. The method also includes removing unbound species from vessel 14 such as by pouring off, the fluid in vessel 14. At this point, nucleic acids 27 are purified and bound to nucleic-acid binding surface 94 (FIG. 5D).

In a particular embodiment, a frozen tissue section is placed into a vessel containing a silica filter binding surface. The sample is then treated with a lysis solution containing a mixture of the strong protein denaturing agent guanidinium isothiocyanate, the RNase reducing agent 2-mercaptoethanol, and the lysis reagent Triton X-100. The lysis solution can include ethanol as a nucleic acid precipitant, or the ethanol may be added later in an additional step. The sample is allowed to incubate for a period of time between one second and 24 hours to free the nucleic acids and capture them on the binding surface. The mixture can be stored at this step indefinitely to preserve the nucleic acids. The sample can be washed to remove other cell components and lysis solution away. The wash solution may be evaporated away if it is a solvent. The binding surface can be treated with a blocking agent, such as BSA.

In another embodiment, a tissue sample is placed into a vessel containing, as a binding surface, magnetic beads with an inert coating with bound oligo DT molecules. The vessel also contains a lysis solution comprising a mixture of the strong protein denaturing agent guanidinium isothiocyanate, the RNase reducing agent 2-mercaptoethanol, and the lysis reagent Triton X-100. The sample is allowed to incubate for a period of time between one second and 24 hours to free the nucleic acids and capture them on the binding surface. The mixture can be stored at this step indefinitely to preserve the nucleic acids. Water is then added to at least 60% of the final mixture volume. In some cases, the water is added as part of the lysis solution. The lysis solution contains a sufficient amount of salts ions to promote the hybridization of the nucleic acids to the oligo DT molecules. Finally, the binding surface is treated with one or more wash steps of 90% ethanol, or alternatively with 100% aqueous wash steps containing at least 100 mM tris(hydroxymethyl)aminomethane, lithium chloride salts, or guanidinium salts, to remove unbound species from the vessel.

In another embodiment, an FFPE or frozen tissue section is placed into a vessel containing beads having a polycarbonate binding surface, and also containing a lysis solution comprising a mixture of the weak protein cleaving agent proteinase K, the RNase reducing agent dithiothreitol, the lysis reagent sodium dodecyl sulfate, and placental RNase inhibitor. The lysis solution has a sufficient salt concentration to promote the hybridization of the nucleic acids to the beads. The sample is allowed to incubate for a period of time between one minute and several days to free the nucleic acids and capture them on the binding surface. Finally, the binding surface is treated with one or more wash steps of 90% ethanol, or alternatively with 100% aqueous wash steps containing at least 100 mM tris(hydroxymethyl)-aminomethane or lithium chloride salts, to remove unbound species from the vessel. In some examples, a FFPE tissue is used without the deparrafinization step.

Exemplary vessels can include vessels formed of polypropylene, polyethylene, polystyrene, polycarbonate, fluoropolymer, acrylic, aluminum, stainless steel, ceramic, silicone, silicon, glass, quartz, acrylic adhesive resin, or silicone adhesive resin.

Exemplary nucleic-acid-binding surfaces include silica, silicon, silicon carbide, silicon nitride, metal oxides, polycarbonate, polystyrene, nitrocellulose, cellulose, chitosan, oligonucleotides, oligo DT, or protein nucleic acids, said binding surface being in an immobilized form comprising porous sheets, fiber filters, mesh, rough surfaces, gels, or beads.

### IX. Methods of Detection

FIG. 12 shows a cross-sectional schematic view of creating a map of the positions and concentrations of target nucleic acids 23 within manipulation platform 10. A source of excitation light 80 shines light 81 onto the target molecules (not shown) in manipulation platform 10. Target nucleic acids 23 have either previously been tagged with a fluorescent dye or are themselves fluorescent. Fluorescent light 82 emanates from those chambers 30 that contain target molecules 23, and this light is detected by detector 83. An example of such a system, into which manipulation platform 10 can be placed, is the Typhoon 9410 Imager by GE Healthcare. Molecular maps can be created because only those wells containing the target emit light, and the intensity of the fluorescent light is proportional to the number of target nucleic acids 23 in micro-scale chamber 30. By using different colors of fluorescent tags, multiple target nucleic acids 23 within chambers 30 can be visualized simultaneously. Simultaneous manipulation of multiple targets is referred to as “multiplexing”.

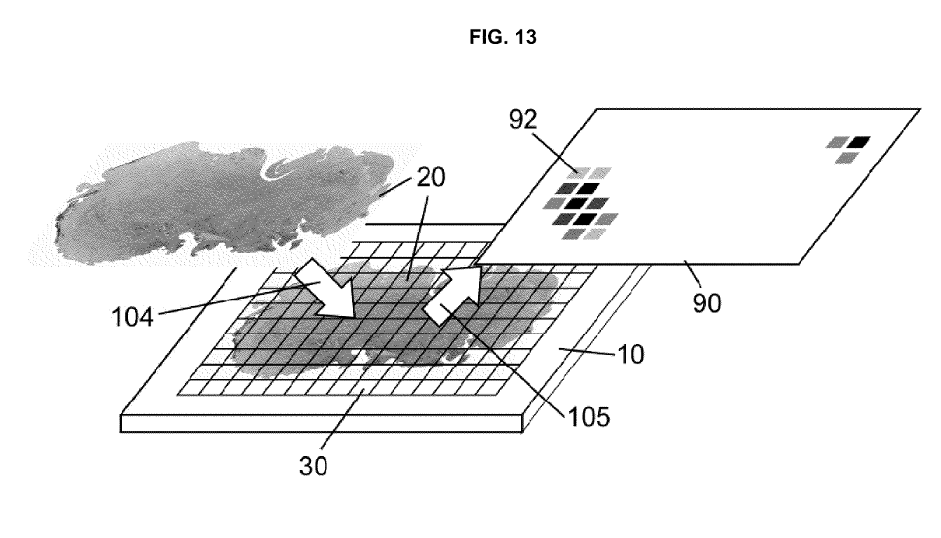

FIG. 13 shows a schematic illustration of a method for creating a molecular map in which nucleic acids 23 are transferred from biological sample 20 to manipulation platform 10, and are then amplified within chambers 30 by PCR. The amplification products are then transferred out onto a capture membrane 90 for staining and detection. The location and intensity of stains 92a creates a 2D spatial map of the positions and concentrations, respectively, of nucleic acids 23 in the original biological sample. This procedure for the creation of a molecular map can be used when the concentration of target molecules within the tissue is too low to be visualized without amplification.

Capture membranes that can be used include one or more LES membranes and nitrocellulose membranes. It is possible to treat such membranes with a binding agent, or a stack of membranes each with a different binding agent, to bind a particular product of interest prior to transferring the manipulated molecules onto the membrane or membranes. The manipulated molecules can be stained after transferring them onto the membrane(s) to visualize their positions.

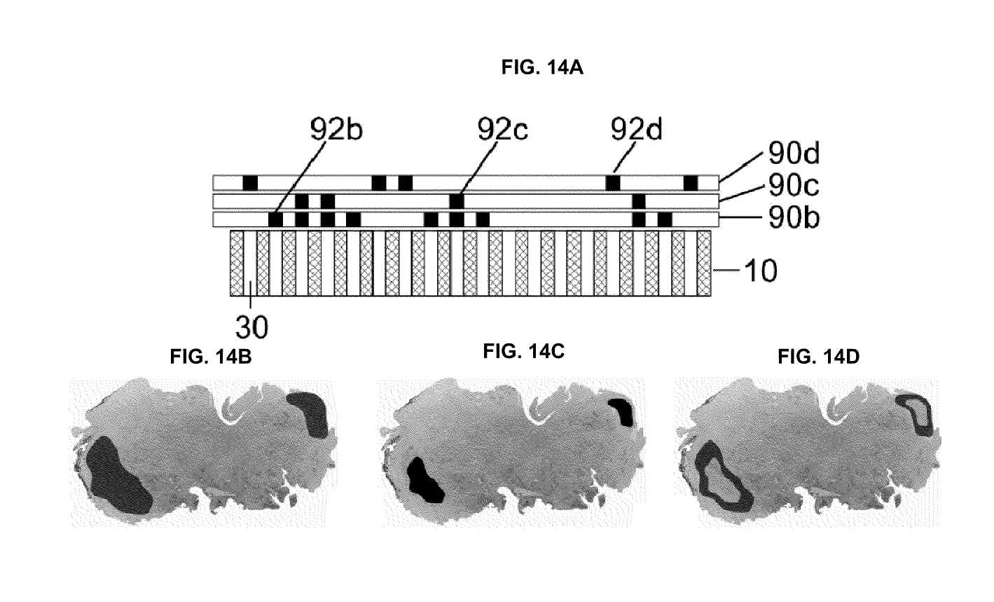

FIG. 14A shows a schematic illustration of the vertical transfer of nucleic acids 23 out of manipulation platform 10 and onto stack of LES membranes 90b, 90c, and 90d. The 2D spatial information of nucleic acids 23 in chambers 30 is retained during this transfer because of the vertical pores in LES membranes 90b-90d. Each membrane in the stack can be treated to capture a different target molecule. After capture, nucleic acids 23 can be stained to create a map of their positions. Multiple targets can be manipulated simultaneously within manipulation platform 10, allowing the subsequent creation of multiple maps at the same time. This is illustrated in FIGS. 14B-D, which show three hypothetical maps of molecules trapped on three membranes after target molecules have been stained and imaged, and these images overlaid onto an image of the original 2D tissue sample. The retention of 2D spatial information throughout the process of transfer to manipulation platform 10, molecular manipulation (including processes such as treatment with proteinase K and PCR), and transfer onto LES membranes allows one to make this correspondence between the molecular maps and the tissue morphology and histology.

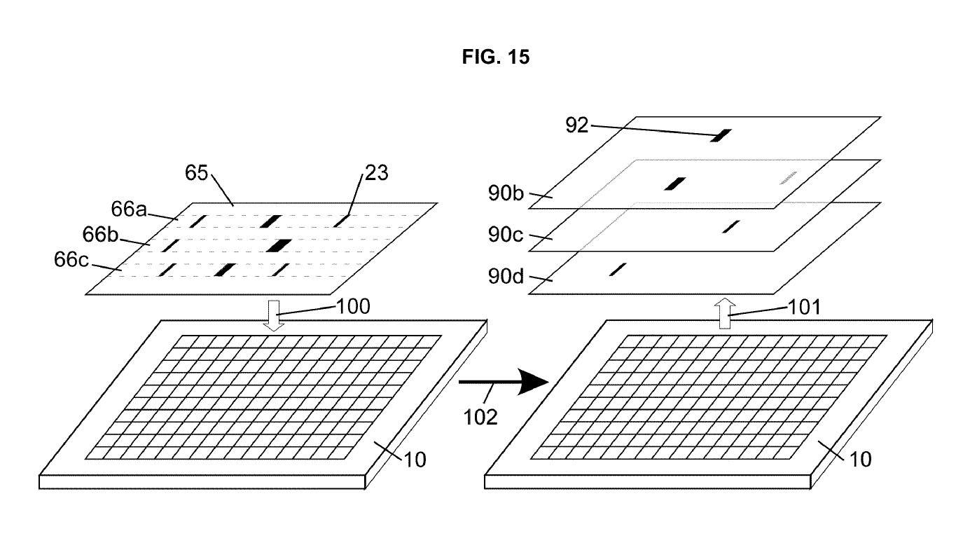

FIG. 15 illustrates of an exemplary method of transferring nucleic acids, manipulating such nucleic acids followed by detecting the manipulated nucleic acids. First, nucleic acids 23 are transferred vertically 100 (represented by the arrow) from gel 65 into manipulation platform 10. For illustration, three samples containing nucleic acids 23 have been loaded onto three lanes of gel 66a, 66b, 66c, and nucleic acids 23 separated on gel 65, for example by size using electrophoresis.

Amplification reaction 102, for example PCR, is performed in manipulation platform 10 on three target nucleic acids. The amplification products are transferred 101(represented by the arrow) to stack of capture membranes 90i, 90j, 90k, each of which is treated to capture a different target nucleic acid. The positions of nucleic acids 23 can be visualized by staining or radioisotope labeling 92.

### EXAMPLES

### Example 1

### Manipulation Platform

This example describes manipulation platforms that can be used with any of the methods and systems disclosed herein as well as other methods and systems known to those of ordinary skill in the art.

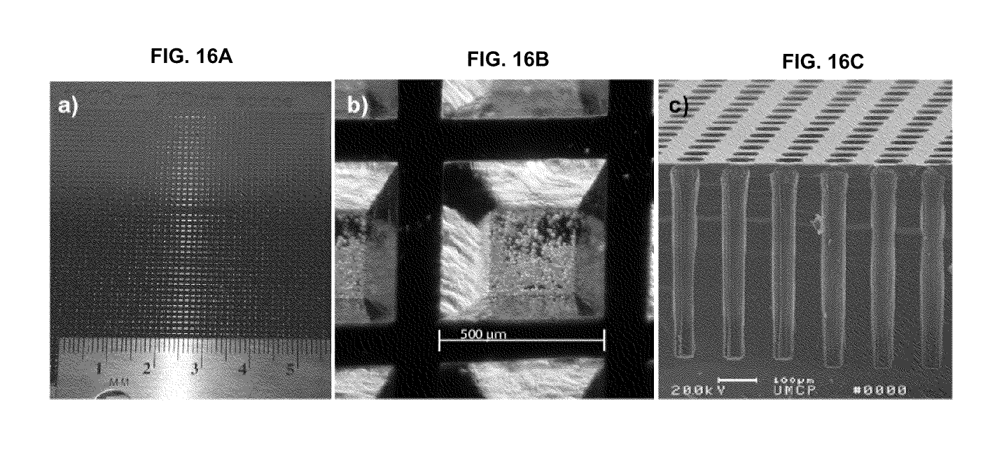

FIG. 16A-C show images of manipulation platforms with chambers in the form of wells in accordance with this disclosure. FIG. 14A shows an overhead view with a ruler for scale in cm of an array of micro-scale wells etched into a silicon (Si) wafer. FIG. 16B shows a close-up, overhead view of a micro-scale well with a 500 μm opening at the top surface that was etched into a silicon wafer by anisotropic wet etching in a solution of potassium hydroxide (KOH). This produces pyramidal-shaped pits. The bottoms of the pits are flat because the etch was stopped before the walls converged.

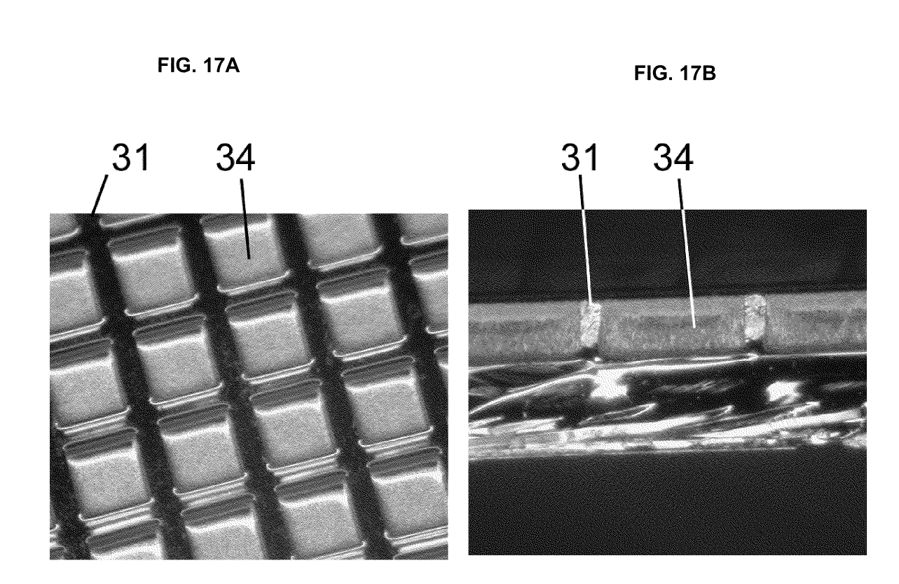

FIG. 16c shows a scanning electron microscope (SEM) cross-sectional image showing micro-scale wells approximately 100 μm in diameter etched by deep reactive ion etching (DRIE), also known as the Bosch process, into a silicon wafer. Assuming a 10-20 μm diameter for the size of a typical cell, wells of 100 μm will contain nucleic acids from a localized population of just 25-100 cells. The micro-chambers are spaced apart by 100 μm, yielding 2500 wells/cm², and a spatial resolution of ˜200 μm considering the dead space. This substrate has well or vial-style micro-scale chambers that are round. This etch method results in substantially vertical sidewalls. It is known to those in the art that aspect ratios of 30:1 can be achieved using DRIE; for example, for a 500 μm thick substrate, this would correspond to a well diameter of about 20 microns. FIGS. 17A and 17B shows another array of DRIE-etched well-style micro-chambers 32 separated by walls 31.

The resolution of the device was determined by the spacing of the chambers. Silicon wafers are attractive as micro-chamber substrates because small, high aspect ratio wells (for example 500 μm deep and 100 μm on a side) can be achieved by DRIE. A process analogous to DRIE has recently been demonstrated for glass, so glass presents an alternative substrate material. Based upon the teachings herein, it is believed that hole size can be decreased even further, thus increasing the resolution of the disclosed device even further. For example, aspect ratios of 20:1 or better are contemplated.

The manipulation platforms in FIGS. 16C and 17 were produced by deep reactive ion etching (DRIE) of double-side polished, 4″ diameter, 500 μm thick <100> Si wafers. SU8-50 (MicroChem), a negative resist, was used as a mask to cover those areas not to be etched. After dehydrating the wafers at 180° C. for 10 minutes, the SU8 was spun onto the wafer, ramping up to 2500 rpm and holding for 40 seconds. The resist was prebaked at 65° C. for 5 minutes and 95° C. for 10 minutes, then cooled to room temperature over 5 minutes. The SU8 was exposed through a mask that included the 100 μm wells and 20 μm wide lines between micro-chamber platforms to aid later dicing. The resist was post-baked using the same procedures as for prebaking. The SU8 was developed (Micro Chem SU8 Developer) for 2 minutes, then rinsed in isopropanol, methanol, and de-ionized water. The wafer was attached to a second “handle” wafer with a layer of spin-coated Shipley 1813 resist. The holes were etched all the way through the wafer by DRIE, using an etch cycle of 10 seconds and a passivation cycle of 6.5 seconds, for a total of 4 hours. The handle wafer was removed in acetone, and the SU8 mask was peeled off.

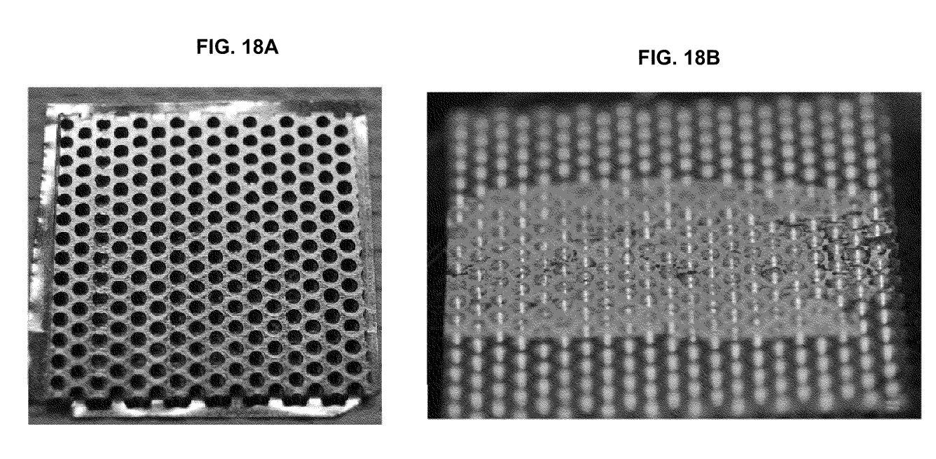

FIG. 18A shows an overhead view photograph of an aluminum (Al) substrate with an array of through-hole style chambers formed by drilling. Each hole is 1.6 mm in diameter. Sheets of this material are available commercially (Perforated Metals Plus). FIG. 18B shows an oblique view photograph of a 14 μm thick section of dried human prostate tissue placed on an Al substrate with an array of millimeter-size wells.

### Example 2

### Surface Coating of Manipulation Platform

This example describes methods for coating the surface of a disclosed manipulation platform.

Typically, pipettes are too small and not accurate enough to put reagents into 100 μm wells. Capillary action is therefore used to draw the fluid into well or through-hole style micro-chambers. The driving force, F, for capillary action is related to the radius of the micro-chamber, r, by F=2π r σ_{LG} cos θ, where σ_{LG} is the surface tension of the liquid-gas interface and θ is the contact angle. If the surface is hydrophobic and the contact angle goes above 90°, there is a negative force for filing the wells. This is the case for Si substrates, necessitating a surface coating to lower the contact angle. Several surface coatings were tested for their ability to facilitate fluid transfer: bovine serum albumin (BSA) (20 mg/mL, BP675-1), Triton X-100 (NC9903183), and Tween 20 (BP337-100), each diluted to 0.2%, 1.0% and 5% in 50% ethanol-water. A 10 μL droplet was coated on one side of a Si manipulation platform with 100 μm diameter micro-chambers and allowed to dry. A 5 μL droplet of water-based food coloring for ease of visualization was placed on the surface, and the percentage of filled holes determined. The 5% BSA and all the Triton X-100 and Tween 20 coatings resulted in complete filling.

The surface coating inhibition limit during PCR was also tested by drying the various surface treatments in standard PCR tubes. None of the surface treatments inhibited PCR in up to 5% tested concentrations. Studies indicated that the use of 5% BSA added to the reagents allowed the transfer of reagents into the micro-chambers and did not interfere with the PCR processes.

### Example 3

### General Methods

This example describes the sequences, concentrations and PCR Protocols used for a number of the Examples provided below.

Several primer and probe sequences were fabricated and are presented below. Primers and probes were ordered from BioSearch Technologies (Novato Calif.). All sequences are listed in the 5′ to 3′ order. All primers were ordered desalted, all probes were ordered with HPLC purification. Unless otherwise specified, PCR consisted of the following conditions. Primers were diluted to 500 nM and probes were diluted to 250 nM in the final mix. Two one-step RT-PCR mixes were used and performed identically. Mix one was the Verso one-step kit (Thermo Scientific), and mix two was the AgPath-ID one-step kit (ABI). Both were mixed according to the manufacturer's protocol. Final PCR volumes were 10 μL. PCR was performed in an ABI 7500 real-time imaging instrument. Cycling conditions for the first cycle were 10 minutes at 50° C. and 10 minutes at 95° C., and for the remaining 40 cycles they were 95° C. for 10 seconds and 60° C. for 30 seconds.

GYS2 Probe set
(Mouse Liver-Accession NM_145572)
Forward Primer:
(SEQ ID NO: 1)
GCCAGACACCTGACACTGA.
Reverse Primer:
(SEQ ID NO: 2)
TCCGTCGTTGGTGGTGATG.
Probe:
(SEQ ID NO: 3)
CalFluorOrange560-TTTCCAGACAAATTCCACCTAGAGCCC-
BHQ1.
Product Size: 73 bp.
Specificity: mRNA/DNA
KCNJ1 Probe set
(Mouse Kidney-Accession NM_019659)
Forward Primer:
(SEQ ID NO: 4)
GGCGGGAAGACTCTGGTTA.
Reverse Primer:
(SEQ ID NO: 5)
GTGCCAGGAACCAAACCTA.
Probe:
(SEQ ID NO: 6)
FAM-AAGCACCGTGGCTGATCTTCCAGA-BHQ1.
Product Size: 67 bp.
Specificity: mRNA/DNA
HPRT Probe set (Mouse Housekeeping
(All Tissues)-Accession) NM_019659)
Forward Primer:
(SEQ ID NO: 7)
GCAAACTTTGCTTTCCCTGG.
Reverse Primer:
(SEQ ID NO: 8)
ACTTCGAGAGGTCCTTTTCACC.
Probe:
(SEQ ID NO: 9)
Quasar-670-CAGCCCCAAAATGGTTAAGGTTGCAAG-BHQ-2.
Product Size: 85 bp.
Specificity: mRNA
BACT Probe set (Human Housekeeping
(All Tissues-Accession NM_013556)
Forward Primer:
(SEQ ID NO: 10)
GGACTTCGAGCAAGAGATGG.
Reverse Primer:
(SEQ ID NO: 11)
CAGGTCTTTGCGGATGTC.
Probe:
(SEQ ID NO: 12)
FAM-TCCTTCCTGGGCATGGAGTC-BHQ1
Product Size: 312 bp (DNA) 217bp (RNA).
Specificity: mRNA/DNA

Human HPRT1 Probe set (when human normal mRNA is specified). This probe set was purchased from Applied Biosystems, part Hs99999909_m1, amplicon length 100 bp.

### Example 4

### Manipulation and Detection of Nucleic Acids within the Chambers

This example demonstrates successful manipulation and detection of nucleic acids within a disclosed device without crosstalk while maintaining the 2D positional information.

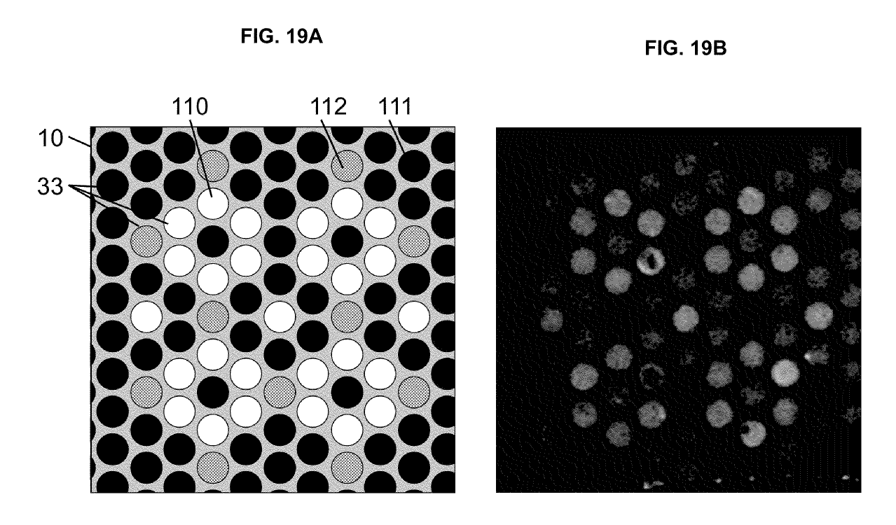

As illustrated in FIG. 19, a target sequence of DNA was manipulated within mm-scale through-hole style wells 33 of an Al substrate, such as shown in FIG. 18A, by amplification using PCR. The target DNA was then detected by transferring the DNA from mm-scale through-hole style wells 33 onto a nitrocellulose membrane and staining with SYBR-gold, as shown in FIG. 19B. The DNA was loaded into wells 33 with a pattern, positive controls 110 (white circles), negative controls 111 (black circles) and dye 112 (crosshatched circles) in FIG. 19A. This pattern is clearly seen in FIG. 19B, showing that the 2D spatial relationship of the DNA in the mm-scale through-hole style wells 33 was retained during PCR and upon transfer to the nitrocellulose. FIG. 19B is an example of a 2D spatial map of a predetermined molecular characteristic, in this case a target DNA sequence.

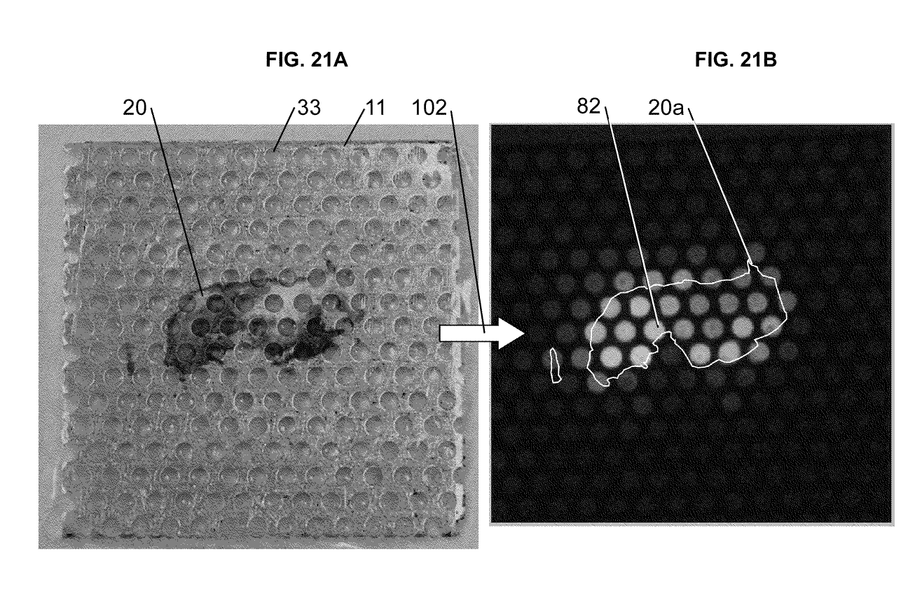

FIG. 21 demonstrates that target nucleic acids can be provided to Al manipulation platform 11, freed from sample 20, and transferred into manipulation platform 11 while maintaining the 2D spatial relationship of the transferred material relative to the original sample, and can then subsequently be manipulated and detected while still maintaining the original spatial relationship they had in the original sample. A description of this manipulation platform and method for its use was described in Armani et al., (Lab Chip, 9 (24): 3526-3534, 2009), which is hereby incorporated by reference in its entirety.

Manipulation platform 11 was made of Al and had through-hole style chambers 33. Exemplary Al manipulation platforms were constructed by obtaining sheets of perforated aluminum, cutting them to size, cleaning them, and attaching an aluminum foil seal to one surface. Perforated aluminum (alloy 3003H14, Perforated Metals Plus) was obtained in 30.5×30.5 cm² sheets 1.27 mm in thickness. The 1.6 mm through-holes had a 2.38 mm staggered center-to-center spacing. They were cut into 3 cm square pieces, for a final cost each of 12¢. The pieces were cleaned with a 1% aqueous bleach solution, followed by a boiling water bath, a 100% ethanol bath, and air drying. The pieces were glued on one side to aluminum foil using a temperature-activated polymer glue (Matrix Technologies 4419) and application of 100 pounds of force (lbF) at 95° C. for 1 minute.

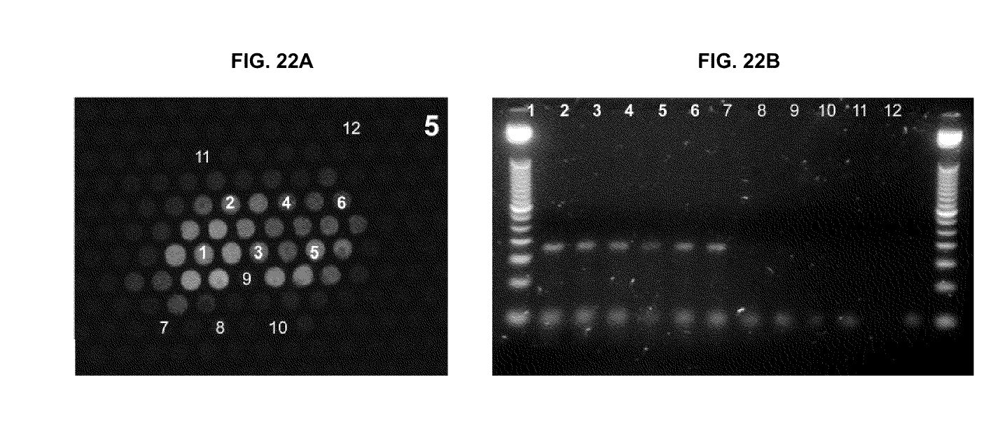

The protocol for mapping the position of the strand of target DNA in FIG. 21 was as follows.
1. Al manipulation platform 11 was dipped into a solution of 2% low melting point molten agarose at 50° C. Surface tension caused molten agarose to fill the chambers.
2. The bottom side of Al manipulation platform 11 was covered with a 25 μm thick adhesive-backed fluoropolymer (FEP) film (McMaster-Carr part number 5805T11).
3. Al manipulation platform 11 was left to cool at room temperature for 10 minutes until the meniscus of the fluid inside micro-chamber wells 30 was clearly concave by visual inspection. (A convex meniscus protrudes during the freezing step and prevents the sealing film from sticking securely).
4. A breast tumor tissue sample, with dimensions of 5 mm×5 mm×15 mm, was unfrozen and dipped in an Eosin Y (0.05% w/v) staining bath for 2 minutes. It was immersed in 70% ethanol for 5 minutes and in 15% ethanol for 15 minutes, and then embedded in OCT tissue fixative on dry ice for 10 minutes.
5. The Eosin-stained breast tumor sample was sectioned to 12 μm thick slices.
6. The top surface of manipulation platform 11 was brought into contact with 2D tissue sample 20 (tissue section), thereby providing 2D tissue sample 20 to manipulation platform 11.
7. Two-dimensional tissue sample 20 and platform 11 were covered by FEP film and imaged. To mark the tissue orientation on the substrate, a diagonal and a square notch were cut out of the sealing film covering the tissue.
8. Manipulation platform 11 was placed with the top-side down on a thermocycler and compressed with 150 lbF to express (push) the tissue overlying the chambers into the chambers, thereby transferring the target nucleic acids, in this case DNA, from 2D tissue sample 20 into the multiple chambers.
9. Manipulation platform 11 was heated to 95° C. for 5 minutes to redistribute the agarose over the tissue, then cooled to 0° C. for 10 minutes. The FEP film on the non-tissue containing bottom surface was removed and the agarose was dehydrated at 95° C. for 5 minutes.
10. DNA elution was performed by manually pipetting a 1 mg/mL proteinase K solution at 2.4 μL into each well of the platform. The platform was frozen at −20° C. for 10 minutes, sealed with a new FEP film, and returned to the thermocycler with 150 lbF compression. It was subjected to 65° C. for 30 minutes to digest tissues/nucleosomes and then 95° C. for 5 minutes to inactivate the enzyme. This treatment freed the target DNA nucleic acids from the sample matrix.
11. The agarose was again solidified and dehydrated.
12. PCR super mix was manually pipetted at 2.4 μL into each well. Supermix contained standard buffers and was adjusted to 200 mM primers for a GAPDH-166 nt genomic DNA target, 5% w/v BSA adjuvant, 60 U/mL Taq polymerase and 2.75 mM MgCl₂.
13. PCR thermocycling was performed with 95° C. for 2 minutes, and then 35 cycles of 95° C., 56° C., 72° C. for 10, 10, and 15 seconds, respectively, followed by a final 72° C. step for 2 minutes and a 0° C. step for 10 minutes.
14. Staining of the DNA was performed by pipetting 300 μL of a 10× dilution of SYBRGreen-I dye on the exposed agarose gels inside the wells. After 5 minutes, a signal was imaged with a CCD camera with shutter speed of 1/15 of a second. Thus, nucleic acids 23 were placed into an environment that allowed subsequent manipulation and detection of nucleic acids 23 while maintaining the 2D spatial relationship of nucleic acids 23 relative to those in the original sample.
15. To validate that the fluorescent signal represented the target amplicon, agarose plugs were removed from 6 positive-signal wells and 6-negative signal wells, diluted 1:100 with water (to dilute the agarose and Eosin Y), melted at 95° C. for 10 minutes, and subjected to PCR for 8 cycles (about 110-fold amplification at 90% efficiency). 2.4 μL of each product was mixed with 8 μL of gel loading buffer and run on an electrophoresis gel. The results are shown in FIG. 22. A band with a molecular weight corresponding to the desired amplicon was present in the lanes loaded with the contents of the fluorescing chambers, and was absent in the chambers that did not fluoresce.

Thus, FIGS. 21 and 22 demonstrate the ability of the disclosed device to be used in the mapping of DNA in a tissue. The GAPDH-166 nt genomic DNA target in a breast tumor tissue section was amplified using the test protocol given above. This study produced a map showing the location of the tissue; the negative control is the area not covered by tissue. The detection method used Sybr-Green I, which stains all double-stranded DNA. FIG. 21A shows a photograph of a tissue (stained for visualization with Eosin Y) on a manipulation platform, and FIG. 21B shows the Sybr-Green DNA signal post-amplification; the original tissue position is indicated by the white contour line. The amplification results were validated on a gel (FIG. 22B). In wells underlying the tissue (lanes 1-6) there was a bright band for the 166 nt product, while negative wells (lanes 7-12) had no amplification of the 166 nt target and only weak bands of ˜50 nt primer-dimers, an artifact of PCR. Note that well 9 was directly adjacent to vials covered by tissue, but was not cross-contaminated. This also shows that the disclosed device, system, and methods can reduce or even prevent cross-talk.

### Example 5

### Methods for Providing a 2D Tissue Sample to a Manipulation Platform

This section describes a method of providing the 2D tissue sample to a platform having a plurality of chambers while maintaining the relative 2D spatial relationships of the nucleic acids in the original sample, which is one step of the disclosed method.

To preserve the relative locations of the cells, frozen tissue was transferred directly onto the manipulation platform immediately after sectioning. In this particular example, a human prostate specimen section of 40 mm×20 mm was fixed in O.C.T. compound (Tissue-Tek 4583) and sectioned using a cryostat microtome. Tissues were sectioned to 4, 8, 12, 16, 20, and 30 μm thickness. Following a standard procedure, each section was spread out to flatten it on an internal cryostat surface using a soft brush. A manipulation platform was placed on top of the tissue, hole-side down. Since the platform was at room temperature, the tissue and the O.C.T. fixative compound melted locally and adhered to the platform. Examination of the shape and size of the tissue indicated that the tissue transferred without any significant kinks, folds, or holes. This study demonstrated that a frozen tissue section adhered to a manipulation platform while maintaining the overall 2D shape and size.

### Example 6

### Methods of Transferring DNA from Tissue Sections into Chambers

This example describes methods used to transfer target nucleic acids from a sample having 2D spatial information into the multiple chambers of the manipulation platform while maintaining the 2D spatial relationship of the transferred material relative to the original sample.

i. Transfer target nucleic acids using pressure. To transfer target molecules from a 2D tissue sample into a manipulation platform, the manipulation platform was flipped tissue-side up and left at room temperature, allowing the tissue to dry. The dried tissue was barely visible to the eye on these platforms, but closer examination revealed that the tissue above the wells either remained suspended above them or adhered to the inside of the wells. A schematic of this transferring procedure is provided in FIGS. 3a and 3b. After the addition of sealing layer 153, pressure 41 was applied to the top face of manipulation platform 10 using pressure platform 40, pushing tissue 21 overlying the chambers into chambers 30. Tissue 22 that did not overlie a well remained on outer surface 31 of manipulation platform 10. After this transfer step, nucleic acids were freed from the tissue by treatment with proteinase K. The system used for transferring nucleic acids 23 from sample 20 into micro-well chambers 30 is illustrated in FIG. 3c.

ii. Transfer target nucleic acids using electrophoresis. In addition to transferring nucleic acids from a tissue section into a manipulation platform by pushing the overlying tissue into the wells during pressure sealing, and then treating the tissue with PK, electrophoresis can be used to selectively transfer only the nucleic acids into the wells. Electrophoresis is well known in the art, and has been shown to move nucleic acids, which are negatively charged, toward the anode. This approach can be used for protein, mRNA, and DNA transfer. An example of an electrophoresis apparatus that can be used to transfer nucleic acids 23 from sample 20 into micro-well chambers 32 is illustrated in FIG. 8 (which is described in detail above). As illustrated in FIG. 8, sample 20 was positioned over gel 61 overlying manipulation platform 10, sample 20 covered with second gel 62, treated with proteinase K, and heat-treated to release the DNA by digesting sample matrix 26. Next the DNA is transferred to substrate micro-well chambers 30 by electrophoresis through gel 61, thereby purifying it from proteins, nucleases, etc. The nucleic acids are then amplified within protein-free conditions inside the micro-chambers. All of these procedures help provide a clean, tissue-free environment for the subsequent PCR while allowing nucleic acids 27 with a high purity to be isolated. A similar procedure can be followed for mRNA, but additional steps may be included to prevent degradation of the RNA by nucleases.

In particular, for loading DNA into a manipulation platform by electrophoresis the following method is used: 1) cover the front surface of an agarose-filled substrate with a polyacrylamide or agarose gel; 2) transfer the tissue section directly onto the gel layer; 3) add a solution of PK to the tissue; 4) cover with additional polyacrylamide; 5) cover the surface with water to prevent evaporation; 6) heat the substrate to 65° C. for 30 minutes to elute the DNA followed by heating the substrate to 95° C. for 30 minutes to inactivate the PK; if smaller DNA fragments are required, place the gel with tissue in a buffer containing restriction endonucleases overnight at room temperature; 7) sandwich the substrate in buffer-soaked paper; 8) perform electrophoresis to transfer the nucleotides into the wells, using the gel as a filter; 9) adding tris-acetate buffer to the assay; and 10) calibrate the electrophoresis time using a DNA ladder (if the electrophoresis time is too short, the nucleic acids may not make it into the wells, and if too long, they could pass through the substrate). To ensure that the nucleic acids, such as DNA, RNA or proteins, remain within the wells, voltage is applied directly to the substrate (instead of to an electrode behind the substrate). This can include depositing a metal film on a manipulation platform for the case of oxidized Si. Alternatively, a nucleic acid-capturing membrane can be placed on the back of the platform, and allow the material to diffuse from there back into the wells. If the nucleic acids are too entangled to migrate, an enzymatic digest, such as a nucleotide restriction digestion for nucleic acids, is used.

iii. Transfer target nucleic acids using suction. FIG. 9 provides an additional example of transferring nucleic acids into micro-chamber wells 32 of manipulation platform 10, in which suction 45 is employed. Two dimensional biological sample 20, encased in a gel 61, 62 and digested to free the nucleic acids from biological sample, is placed over manipulation platform 10 having through-hole style wells 32 (or 33, not shown). Nucleic acids 23 that have been freed are transferred into wells such as micro through-hole style wells 32 by means of the application of suction force 45 at the bottom of manipulation platform 10. The means for transferring the nucleic acids from sample 20 into micro-well chambers 30 is a suction apparatus.

Although the present examples have provided three specific methods of transferring target molecules into a manipulation platform, it will be apparent to those in the art that many different means for transferring nucleic acids from the tissue sample into the manipulation platform will be efficacious, and all the methods known in the art are included within the scope of the disclosure in addition to those specifically described in these embodiments.

### Example 7

### Methods of Freeing Nucleic Acids from the Sample Matrix

This example describes methods for treating a tissue sample to free the DNA nucleic acids from the tissue sample matrix while maintaining the 2D spatial relationship of the nucleic acids relative to the original sample.

Tissues within the chambers of an Al manipulation platform were lysed by incubation with proteinase K to elute the DNA. A 2.4 μL amount of 1 mg/mL proteinase K (PK) solution was pipetted into each micro-chamber. It was subjected to 65° C. for 30 minutes to digest tissues/nucleosomes and then subjected to 95° C. for 5 minutes to inactivate the PK enzyme. This treatment freed the target DNA nucleic acids from the sample matrix.

In another example, tissues were also lysed to release the genetic material locally. The ability of the lysing agent to release the DNA was confirmed in an Al manipulation platform. To lyse tissues, the method of proteinase K tissue digestion was investigated. Powdered normal prostate tissue was diluted in water to 6.7 ng/μL, and proteinase K was diluted in this solution to 2 mg/mL. The tissue-proteinase K mixtures was pipetted into 12 chambers and incubated at 65° C. for 30 minutes, then the pK was inactivated at 95° C. for 2 minutes.

The samples were then recovered from the chambers and combined, and put into a single well of a second Al manipulation platform to test, by PCR, whether the DNA had been eluted. Once this sample was pipetted into the chambers, it was amplified for detection.

First, primers pairs were mixed together at a concentration of 250 μM per primer, 2.4 μL of each desired primer pair was pipetted into the individual wells, and the wells were dehydrated at 95° C. for 2 minutes. The test tissue sample isolated from PK-lysed prostate tissue and controls were next pipetted into various wells on the substrate, 2.4 μL per well, directly on top of the dried agarose and primers, and heated dry at 95° C. for 1 minute. A genomic DNA control was included on the plate as a dilution series to semi-quantitatively gauge the efficacy of nucleotide elution, as was a negative water control. PCR mix was then loaded at 2.4 μL into individual wells. All the reagents were loaded in a humidified environment so as to minimize the evaporation difference across samples. The PCR SuperMix included a 167 nt GAPDH genomic primer set at 250 μM. This left the wells filled with a dried agarose pellet containing DNA, primers, and PCR SuperMix ingredients. The substrate was then frozen and pressure sealed with aluminum sticky foil. After sealing, the manipulation platform was placed on the thermocycler (MJR PTC-200) surface and covered with 100 μL of mineral oil. PCR was performed with the following parameters: 95.0° C., 1 minute, followed by 25 repeated cycles of 1) 95.0° C., 5 seconds, 2) 56.0° C., 5 seconds, and 3) 72.0° C., 15 seconds, followed by 72.0° C. for 2 minutes, followed by a cool down step to 4° C., 30 minutes. Samples were then individually mechanically extracted from the chambers and visualized on an electrophoresis gel.

Fifty nucleotide (nt) ladders were found in lanes 1 and 7. Lane 2, the negative water control, had no band at 167 nt. Lanes 3-5 had the purified genomic DNA at increasing concentrations, which showed the 167 nt band above 212 pg/μL. The well including tissue also yielded this band in lane 6. Since the intensity of the tissue extract band was about as bright as the one for control DNA at 1.06 ng/μL, a DNA extraction efficiency of at least 2.4% was calculated. This study demonstrated that prostate tissue could be lysed with proteinase K and subsequently amplified in the manipulation platform with significant cross-contamination occurring amongst the chambers. These studies also demonstrate robust PCR in a manipulation platform, in which PCR in a mm-scale well substrate was comparable to PCR in standard tubes.

These studies demonstrate that the proteinase K digestion of the prostate tissue successfully released the genomic DNA, and that DNA from tissue could also be amplified in the manipulation platform.

### Example 8

### Methods to Reduce Reagent Evaporation and Cross-Talk Using Seals

This example describes methods used to prevent or reduce sample evaporation or cross-contamination in the manipulation platform by using seals. During PCR, fluid in the manipulation platform is heated to near-boiling. To prevent or reduce contamination from one micro-chamber to its neighbors, and to prevent or reduce evaporation of the fluid in each chamber, seals can be added to the openings of the chambers.

i. Pressure Sealing a Manipulation Platform.

Studies were first performed to identify a material that was malleable, but was not so viscoelastic (time-deforming under pressure), or did not contain so much adhesive, that the sealing material embedded itself permanently into the holes in the substrate. A number of sealing materials were tested by measuring the fluid loss from a fixed initial fluid volume under the same temperature, pressure, and time. This provided measurements of the total percentage of evaporation 1) before loading and 2) after heating.
a. Pressure Sealing a Manipulation Platform with mm-Scale Wells.

Sealing materials were placed in contact with one side of the substrate. Ten wells were filled with water-containing colored dye by pipette, and the fluid was frozen by placing the substrate on dry ice. The other side of the substrate was covered with the same sealing material. Thermocycling during PCR was then mimicked. The sealed substrate was placed on a thermocycler heating block, covered in 200 μL of mineral oil, covered with a 1″ PLEXIGLAS™ block and a 1 inch aluminum block, and compressed using 100 psi force. The substrate was heated at 98° C. for 15 min, then cooled to room temperature. Temperatures were verified to within 2° C. using a type J thermocouple. The substrate was placed on dry ice for several seconds, the top seal was peeled off, and the substrate was brought to room temperature. To measure the change in fluid volume, the total volume of fluid in the 10 wells was collected with a 30 μL pipette. These studies are summarized in Table 1 below.

TABLE I
Results of testing various sealing compositions.

| Material | | mm-Scale Wells | | | | | |
|---|---|---|---|---|---|---|---|
| Sealing | | Temp. | Fluid | | Micro-Scale Wells | | |
| Material | Description | Accurate | Recovered | Comments | Evaporation | Spreading | Comments |
| Parafilm | thick, | yes | 0% | stuck | N/A | N/A | plugs wells |
| | viseoelastic | | | | | | |
| Microseal A | clear, | yes | 0% | stuck | ~11% | <1% | |
| | proprietary | | | | | | |
| Microseal B | clear | yes | 0% | stuck | N/A | N/A | plugs some |
| | proprietary | | | | | | wells |
| Alum sticky foil | adhesive | yes | 94% | | N/A | N/A | removal |
| | | | | | | | breakage |
| Adhesive FEP | adhesive | yes | 93% | | 0% | ~5% | |
| | fluoropolymer | | | | | | |
| EDPIM rubber | plastic | no | N/A | | | | |
| | rubbers | | | | | | |
| Silicone | medical grade | yes | 94% | slightly stuck | N/A | N/A | Inconsistent |
| Control | NA | N/A | 95% | max pipetted | | | |

As shown, the materials (except one material) reached the desired temperature within +2° C. in 15 seconds. Aluminum sticky foil, adhesive NLP, and thin medical grade silicone prevented evaporation from the mm-scale wells. The other materials adhered more strongly to the thermocycler than the Al platform. Although ˜7% of the water was unaccounted for in these studies, a control study without heating for each sealing material showed that at least 5% of the water was lost in pipetting. Thus, only 1-2% of the water may have evaporated during heating. The lack of lateral spreading between wells was visually confirmed by the addition of dye to some of the wells but not to others. A full demonstration of well isolation is described herein.
b. Pressure Sealing a Manipulation Platform with Micro-Scale Wells.

The same cleaning, compression sealing, and seal removal methods were used as above for the mm-scale well manipulation platform. A pipette was used to spot food coloring onto groups of wells, and the fluid was drawn in by capillary action. The same materials as above were tested with micro-wells, except ethylene-propylene-diene monomer (EDPM) rubber, but before use they were compressed at 100 psi and 98° C. for 15 minutes to reduce their thickness and avoid plugging the wells. Micro-wells were photographed with a digital camera using a macro lens, and the images analyzed with a custom-designed MATLAB script. The results are summarized in Table 2. Microseal A prevented evaporation of reagents from most of the micro-well platform, and it prevented 99.8% of crosstalk.

These studies show that both the mm-scale well and the micro-well external inhibitor substrates can be effectively sealed while they are heated beyond the highest PCR temperature and time. It is therefore possible to isolate reagents in different wells from each other, and to perform PCR cycling or tissue lysis with such sealing methods.

### Example 9

### Methods to Reduce Reagent Evaporation and Cross-Talk Using Gels

This example describes further methods used to prevent or reduce sample evaporation or cross-contamination in the manipulation platform by using gels. To prevent or reduce mixing of the contents of one micro-chamber with the contents of adjacent chambers by the movement of fluids by surface tension when removing or adding a seal, agarose can be added as an immobilizing agent for the reagents and the nucleic acids. If the fluid is entrapped inside a gel, then the contents of the micro-chamber cannot flow freely out of the chamber.

Low melting point (LMP) agarose was obtained from two commercial sources (Lonza 50081, Promega V2111). LMP agarose was tested at 3%, 1.5%, 0.5% wt/vol in 30 mL water with a small amount of food coloring for visualization. The mixture was prepared in a sterilized beaker, covered with a watchglass, and microwaved at 800 W for 60 seconds, then cooled to 40° C. A 20 μA research pipette was used with a filter-tip to draw out the molten agarose.

i. Loading of a Manipulation Platform with mm-Scale Wells. A 2.4 μL volume of melted agarose was pipetted directly into each well. The substrate was covered on one side with a clear adhesive-backed fluoropolymer sealing film (McMaster Carr 5805T11). Agarose up to 3.0% could be solidified; these cylindrical plugs could be physically removed.

ii. Loading of a Manipulation Platform with Micro-Scale Wells. A BSA-pretreated substrate with micro-wells (prepared as described in Example 2) was spotted with the agarose mixture and covered on both sides with the same sealing film. A 1″ PLEXIGLAS® substrate was placed on top of the plate with a 200 gram weight to create even spreading. The micro-well substrate was placed in a 4° C. refrigerator for 15 minutes to solidify the agarose. The agarose could be seen in all of the micro-wells for concentrations up to 1.5%. The 3.0% gel dried too quickly to be drawn into the platform by capillary action.

To ensure that the agarose was compatible with PCR, the reaction was tested in the presence of agarose in a standard PCR tube. For each brand, 10 μL of 3%, 1.5%, 1.0%, and 0.5% weight/volume agarose was pipetted into a standard 250 μL thin-walled PCR reaction tube and dried at 105° C. for 10 minutes. As controls, each tube was loaded with positive control cDNA, and each gel at 3% plus a non gel sample was loaded with no sample as negative controls. When PCR was completed, the tubes were removed after the 72° C. step, and a 2.4 μL volume of the final molten agarose-containing reaction product was pipetted into a PCR tube with 8 μL of gel-loading buffer at 65° C., incubated for 10 minutes, and then 7 μL was spotted into a standard agarose electrophoresis setup for visualization. The PCR products were run on a 2% electrophoresis gel. A 587 nt GAPDH band product was generated in samples with either type of agarose in an identical location to the positive control (+), without agarose. Three negative control lanes showed no false-positive signals.

These results demonstrate that standard PCR quality was not unduly affected by the addition of agarose up to 3.0%; the intensity of the bands, as indicated by a SYBR-gold 1× stain, was slightly weaker in the wells containing agarose, but this could be offset by the addition of more Taq® polymerase. Furthermore, it has been shown that a new thermophillic polymerase, DynaZyme II (Finnzymes) can provide even more robust and consistent amplification in the presence of agarose, in particular, without the need for BSA, although BSA can still improve its functionality. These studies also demonstrate that seals can be added or removed and nucleic acids transferred into the wells without mixing.

### Example 10

### Methods for Adding Fluid to the Manipulation Platform

This example describes methods that can be used to allow additional fluid to be added into the disclosed manipulation platform substrate.

To allow additional fluid to be added into the chambers, the agarose in the chambers was dried to reduce its volume to <3% of its hydrated volume. To do this, the top-surface seal was removed, and the platform was heated to 85° C. for 2 minutes and then 105° C. for 2 minutes; gradual heating prevented steam from ejecting the agarose cylinders from the chambers. In later steps, the dried agarose was rehydrated by the addition of fluid and heating to 90° C. Fluids were loaded in a humidified environment so as to minimize the evaporation difference across samples.

### Example 11

### Methods of Using the Manipulation Platform for Amplifying Methylated DNA

This example describes methods that can be used to amplify methylated DNA with a disclosed manipulation platform.

To amplify only the methylated DNA, a modified in-tissue bisulphate method can be used. DNA can first be eluted with proteinase K (PK), and then bisulphite modification can be performed using the EZ DNA Methylation Gold-Kit (Zymo Research). The modified DNA can be transferred into the wells by electrophoresis and selectively amplified by PCR using primers for the CpG codons. If the bisulphite modification is non-uniform across the tissue, the DNA can be transferred into the multi-well platform and the modification done under these cleaner conditions. The DNA can then be transferred through a micro-column to a second manipulation platform, appropriately registered to the first.

With the disclosed manipulation platforms, 3D maps may be produced in days instead of months. Regions of tissue can be identified independently as methylated to a particular level or not, either by LCM and/or by standard tissue microdissection followed by PCR in 96-well plates. Using this information as well as a titration series and positive and negative controls, the number of PCR cycles in the wells can be calibrated to a desired threshold. The concentrations of the various components of the PCR mix can be optimized for the new PCR conditions.

ANOVA and F-tests can be used to discriminate whether the proportion of methylation in the sample, such as in a tumor, changes laterally, vertically between slices, and across subjects. Data can be suitably transformed (such as with an arc-sin transformation for the lateral interactions) to yield approximately normal distributions so that the ANOVA and F-tests can be applied.

Based upon the teachings herein, high resolution mapping of the GSTP1 promoter methylation can be performed, as illustrated in FIG. 8. High resolution allows various cell types to be compared. For example, this method can be used to compare methylation in stromal cells with methylation in epithelial cells.

For prostate samples, data visualization can be folded into an existing “prostate 3D reconstruction database” (K. A. Cole, D. B. Krizman, and M. R. Emmert-Buck, “The genetics of cancer—a 3 D model,” Nature Genetics, 21, 38-41, 1999). In that database, investigators are initially presented with a general overview of the whole prostate and multiple transverse views at various levels of the gland to orient the investigators to the number, extent, and anatomic location of tumors, hyperplasias, and pre-malignant lesions. Transverse sections are annotated with the types and location of histopathology present as well as the experiments that have been performed on each cell population. The viewer can then click on a cell population of interest to view an image of the dissected cells and concurrently query the molecular database. Molecular data obtained using the disclosure herein can be added to the transverse sections. The investigator would then be able to click on a section to see methylation or one or multiple mRNA maps colored and overlaid on top of the histology views.

These studies allow methylated DNA to be amplified first on the mm scale (overall methylation levels) and then at 200 μm resolution (distinguishing stroma from epithelium). It is also expected that these studies can be used to answer questions concerning the nature, extent, and case-to-case variability of the methylation field in prostate cancer.

### Example 12

### Methods of Quantifying mRNA

This example describes methods for quantifying mRNA.

Areas of a sample, such as a tissue sample, that have over- or under-expressed genes can be identified by detecting mRNA copies at some threshold level. The threshold level can be determined by the amount of product of a housekeeping nucleotide, which is controlled by number of cycles. Dilution ladders can be used to determine the number of cycles needed to see copies at the threshold level. Quantitative real-time methods (CT values, point of curve inversion) can also be used to determine the approximate initial expression level of mRNA in single wells of a multi-well substrate. Another quantification method is to limit the concentration of amplification primers, which slows down the saturation of amplicons, enabling a direct end-point fluorescent measurement which can be correlated to the amount of starting product. Furthermore, samples below a certain concentration naturally plateau at lower fluorescence levels.

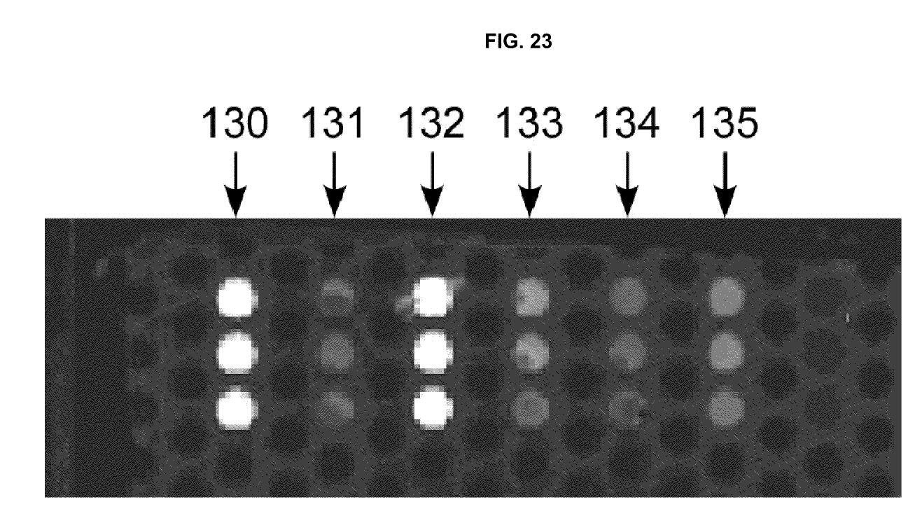

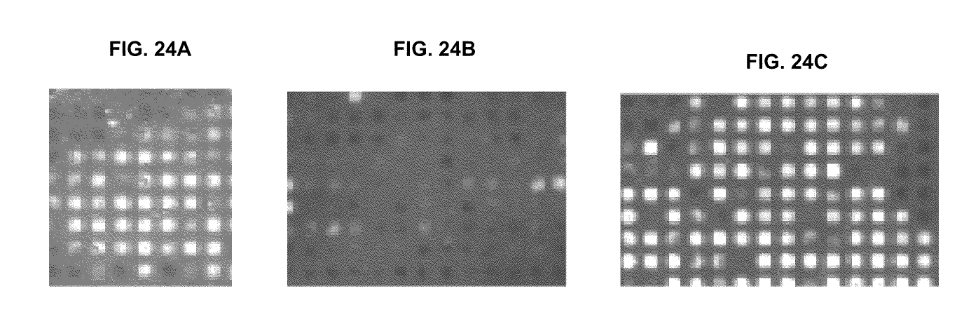

Exemplary results with different concentrations of DNA are provided as FIG. 23, FIGS. 24A-24C, and Table II. FIG. 23 illustrates successful PCR when utilizing disclosed Al manipulation platform 11 with 1.6 mm diameter chambers. FIGS. 24A-24C illustrate successful PCR in a disclosed manipulation platform 10 formed with silicon with 100 I-1 m diameter chambers when employing a dual-labeled fluorogenic probe called a TaqMan® probe. FIG. 24A shows a strong positive control with CT value of 24 (130), FIG. 24B a negative control with no CT value, and FIG. 24c a strong positive control with CT value of 30 (131). The image pixel intensities were directly related to the presence or absence of DNA.

TABLE II
Quantification of PCR products using ImageProPlus imaging software.

| Hole | 1 | 2 | 3 | 4 | 5 | 6 | 7 |
|---|---|---|---|---|---|---|---|
| Image Mean | 44,000 | 11,300 | 41,500 | 15,500 | 12,900 | 15,000 | none |
| CT Value | 24 | none | 29.69 | 49.2 | None | 48.8 | none |

These data suggest that quantitative real-time methods (CT values, point of curve inversion) can be used to determine the approximate initial expression level of mRNA in single wells of a multi-well substrate. These data also suggest that such methods can be used to generate multi-dimensional maps of mRNA with high resolution.

### Example 13

### Methods of Generating Multi-Dimensional Maps

This example describes methods of generating a multi-dimensional map of specific nucleic acids in a prostate sample by employing a disclosed manipulation platform.

Based on the teaching herein, the disclosed manipulation platforms can be used to generate a multi-dimensional map of specific nucleic acids present in a sample, such as a sample obtained from a subject believed to afflicted by a disorder or disease. In this particular example, the sample is a prostate tissue sample. For the manipulation platform with mm-scale wells, the back of the substrate is sealed and the wells are filled with an agarose gel as described herein, but with proteinase K (PK) added. A frozen prostate tissue section of 4-8 μm in thickness is cut and transferred directly onto the substrate with mm-scale wells. It is contemplated that a fixed tissue rather than a frozen tissue could be used as well. For example, a fixed tissue can be released from the surface if it is hydrated, and can then be transferred to the manipulation platform in the same way as a frozen section by surface tension. The tissue section is pressure-sealed against the top of the substrate and the substrate is heated to 65° C. for 10 minutes to elute the DNA. The substrate is then heated to 95° C. and held at this temperature for 2 minutes to inactivate the PK. The substrate is cooled to 4° C. for 15 minutes to resolidify the agarose. The seal is removed and the agarose dried. PCR reagents are added by pipetting, the substrate is frozen on dry ice, and solidified excess is removed from the surface. The substrate is then re-sealed and placed into thermocycler. Mineral oil is added to the sample, pressured applied to the top of the seal, and subject to PCR cycling. Upon completion of PCR, the substrate is cooled to 4° C. for 15 minutes to resolidify the agarose and then the seal is removed. An LES membrane filter or a treated nitrocellulose membrane is positioned on the top surface of the substrate. The bottom of the substrate is then unsealed, and the substrate and the membranes are sandwiched between buffer-soaked paper and allowed to equilibrate. Electrophoresis is performed to transfer the target amplicons to the prepared detection membranes and the DNA is visualized by a Typhoon 9410 gel, blot, and microarray imager.

Labeling by fluorescent probing can be highly specific because the hybridization probe has a sequence matched to the middle of the amplicon, which is only present if the target was extended. Fluorescent quantification is performed using Image Pro Plus. For nitrocellulose studies, the Image Pro Plus will be used with a standardized image processing script, to determine the range of fluorescence intensities indicating the presence of a target with multiple primer sets with samples run at 25 and 35 PCR cycles. Probing is performed by using standard probe hybridization techniques, including using SYBR® gold staining. If statistical deviations are too large, an in-situ hybridization protocol to the nitrocellulose membrane can be used in place of SYBR® gold staining.

This method provides a multi-dimensional map of specific nucleic acids present in a prostate tissue sample with brighter signals being detected where cells with the targeted amplicon are more concentrated.

### Example 14

### Methods of Mapping mRNA Expression Levels for Multiple Genes Simultaneously

This example describes methods of mapping mRNA expression levels for multiple genes simultaneously.

Hundreds of LES membranes can be stacked on top of each other, each treated to capture a different product. However, it may be difficult to amplify low and high abundance mRNA at the same time, since the number of PCR cycles required to amplify the low-abundance species would result in saturating the high-abundance species everywhere. Therefore, multiplexing can be performed for species of comparable abundance mRNA species, whereby comparable means within a factor of 1000. This is based on the ability to vary the amplification by controlling the primer concentrations, as described below.

The conditions for multiplexing can be established in standard 96-well plates with control mRNA (such as with whole-tissue extract from prostate, available commercially). As with any standard multiplex RT-PCR reaction, several primers can be designed for each target gene, and primer sets chosen for each (based on those having the lowest primer-dimer interaction, no false-priming signals, and product yield), and permutations of primers for all three targets tested until an appropriate multiplex primer set is identified. This optimization is routine because it can be performed in standard tubes with control mRNA. Conditions to achieve desired threshold levels can then be optimized as described above. If desired, semi-nested multiplex primers, where only one primer is designed for each target gene, and each primer forms a set with a fourth primer having a sequence that is common to all 3 targets, can be designed. It is a well accepted practice to control the primer concentration to vary the efficiency of the PCR reaction. Standard primer concentrations are 100-250 μM, but concentrations as low as 50 and as high as 1000 μM can be used. Variations in this range can yield a difference of 15 PCR cycles, or a factor of 5000 in product concentration. In a particular example, the number of cycles can be increased and the concentration of primers decreased for high-abundance species. It is expected that relative abundances within a factor of 1000 can be handled in this manner.

These methods can be used to yield multiplex mRNA amplification (e.g., several mRNAs at the same time on a single platform) for species of comparable (within a factor of 1000) abundance. In particular, 3D mRNA expression maps of up-regulated genes (such as ALCAM, TACSTD1 and nectin) in whole mount prostate cancer tissue sections can be generated by such methods.

### Example 15

### Method of Nucleic Acid Purification

This example describes a method of nucleic acid purification. The nucleic acids include DNA, total RNA (mRNA, ribosomal RNA, and transfer RNA), and nucleic acid sequences longer than 100 nt. The ability to proceed directly from a purified sample to downstream applications such as PCR would streamline the nucleic acid, and particularly RNA, extraction, purification, and detection process, and potentially enable new applications. These may include automated RNA sequencing, automated forensic analysis, clinical testing, and parallel RNA amplification across a tissue section to provide a two-dimensional visualization of genetic changes in a sample. In addition, the streamlined method would help to increase the throughput of individual researchers.

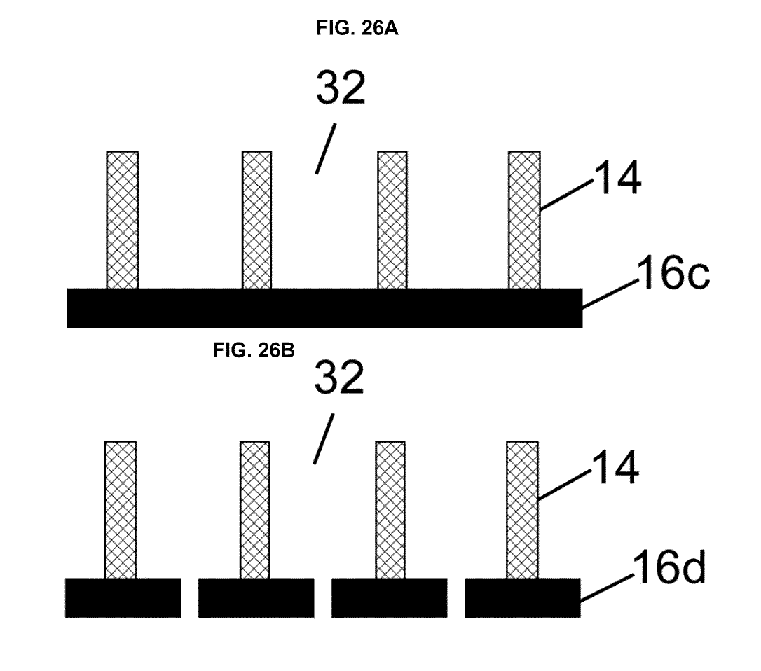

An array of 9 vessels was constructed by drilling 3.125 mm diameter through-holes into manipulation platform 10 of polycarbonate of dimensions 30 cm by 30 cm area by 3.125 mm thickness. Wells having small apertures for fluid draining on bottom surface 16d were created by adding sealing film 16c (25 micron thick polyimide film with an adhesive backing) to bottom surface 16d of manipulation platform 10 and punching a hole of about 500 microns diameter in the center of through-hole chambers 33 (FIGS. 26A and 26B). To clean manipulation platform 10, it was rinse with 100% ethanol liberally and dried at 85° C. for 3 minutes.

Tissue scrapes were manually placed into micro-scale wells 30. As a nucleic acid binding surface, a glass fiber matrix (removed from a PicoPure purification column) was cut into pieces of 2 mm×2 mm One piece of filter was placed inside each micro-scale chamber 30. Holes in bottom sealing film 16c were sealed by layering a second sealing film over the first.

Micro-scale wells 30 of manipulation platform 10 were filled by pipette with a solution of 1 part PicoPure Extraction Buffer (to denature proteins in the tissue and lyse the cell membranes), and one part 70% ethanol+30% water (to cause nucleic acids 27 to adhere to the glass fiber matrix more efficiently than in the absence of such agents). The large openings on the top surface of manipulation platform 10 were covered by a layer of the same sealing film (to prevent evaporation). The substrate was subjected to heating at 45° C. for 30 minutes (to facilitate the reactions). The entire substrate was frozen on dry ice to enable removal of the outermost top and bottom seals without spreading or removal of the well contents. Vacuum suction was applied to the small holes in sealing film 16c on bottom surface 16d for a few seconds to draw the buffer reagents through and out of micro-scale wells 30. This step leaves behind a crude nucleic acid lysate on the silica matrix.

The filter was washed several times using the premixed wash solutions included in the Arcturus PicoPure kit. Wash 1 was added first to the maximum volume of each well, and then removed by vacuum suction. Wash 2 was then added and removed in the same way twice. Note that between wash 1 and 2 additional reagents could be added, such as DNase, if desired. For example, the DNase treatment may enhance the signal-to-noise ratio of fluorescently detected cDNA or prevents non-specific detection of DNA. DNase was mixed with a buffer and water, and then added to micro-scale wells 30 in the amount of 5 μL. The solution was allowed to sit at room temperature for 5-10 minutes. The remaining volume of each well was filled with 12 μL of 70% ethanol in water. This solution was removed by vacuum suction, and the procedure continues on with the two washes of Wash 2. After Wash 2, purified RNA was on the silica matrix.

Manipulation platform 10 was heated to 85° C. for 3 minutes to remove residual ethanol, which may inhibit PCR if present in amounts greater than about 0.1% by volume compared to the well volume. PCR SuperMix with 0.2% by volume BSA was then added. The BSA facilitates the reverse transcription (possibly by preventing the adsorption of reagents, such as DNA polymerase, reverse transcriptase, or PCR amplicons, or by releasing RNA from the surface of the silica filter matrix). This enables first strand synthesis and PCR amplification to occur in the solution directly. The PCR SuperMix included dNTPs, TaqMan primer/probe mix, Taq polymerase, reverse transcriptase, and buffer salts. It may also include preservatives and/or adjuvants. Both faces of manipulation platform 10 were sealed using fresh sealing film, the same type as used previously, and compressed with 150 lbF. The manipulation platform was surrounded by mineral oil to improve thermal contact with the flat heating surface of a PCR machine. Thermo-cycling was performed. Success was determined by imaging micro-scale wells 30 on a Typhoon 9141 imager to see if there was positive fluorescence from the TaqMan probe.

If the amount of RNA is high enough, i.e., greater than 1 ng per filter, then PCR may be successful without the addition of a blocking agent such as BSA.

The PCR SuperMix may include a detection probe. If the thermocycler includes a laser with a 488 nm excitation wavelength and a detection filter of 520 nm, the amount of RNA can be determined quantitatively. A second fluorescent probe, such as Rox, may be included to compensate for differences in well-to-well volume, laser excitation, etc. A detection map can thereby be made directly, with the well contents in situ.

At any step of the process, a preservative or adjuvant may be added to improve the process efficiency, consistency or to enable downstream applications. Known examples of adjuvants are Tween 20, Triton X-100, bovine serum albumin, DMSO, glycerol, sugar, Polysucrose, Ficoll, polyvinylpyrrolidone (PVP), poly ethylene glycol (PEG), and other blocking agents previously referenced. It may also be possible that agarose, polyvinylpyrrolidone (PVP), silane, other wetting agents, emulsifiers, detergents, and blocking agents could be used, as is known in the art.

i. Methods for Providing a Vessel. In an embodiment, the purification of nucleic acids can be performed within the disclosed manipulation platform. In another embodiment, the purification can occur within a single tube or vessel, such as within a well of a multi-well plate or a thin-walled PCR tube.

The material of the vessel may include heat-conducting materials, such as metals, including aluminum, stainless steel, brass, titanium, carbon fiber, or silicon. Alternatively, the material may be a thermal insulator, such as glasses, ceramics, or polymers, the latter including polycarbonate, acrylic, polypropylene, and polyethylene, as well as hydrogels such as agarose, polyacrylamide, or dextran. In most cases the water will be heated by convection and conduction, but it is also possible to heat the water using electromagnetic radiation.

The well diameters and volumes can be virtually any size. For example, the diameter may be 6-mm as found in a standard thin-walled PCR tube, or it may be 100 μm or smaller, as found in the disclosed manipulation platform. The wells can be formed by methods known to those in the art, including drilling, hot embossing, stamping, and etching.

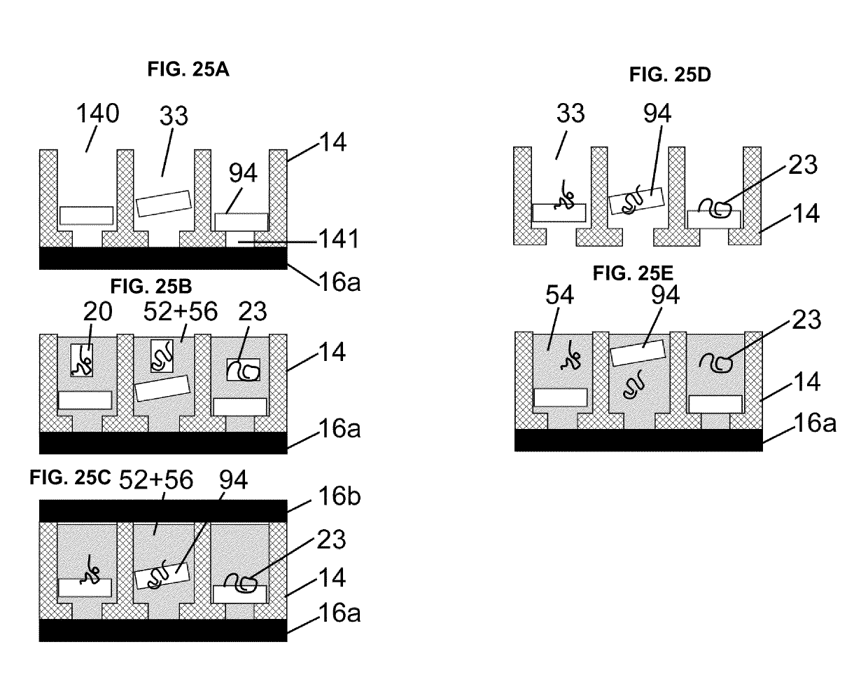

The shapes of the interior of the vessel may include wells, through-holes, and other configurations known to those in the art. The profile of the vessel interior may change with depth to provide narrow passages or cone shapes or apertures (as in FIG. 25).

A large surface area can bind the nucleic acids to give a high yield and capture efficiency for the purification. The nucleic acid binding surface may bind nucleic acids with higher affinity than it binds proteins, lipids, and other tissue components. The nucleic acid binding surface may also be stacked to achieve greater efficiency or higher yield.

In an embodiment, the interior of the vessel itself has a high surface area. For example, the walls of the vessel may be rough. Also, as is known to those in the art, such vessel may comprise a porous material surrounded by non-porous material. In an example, silicon can be etched selectively to produce porous silicon, which has a high surface area. The etch depth can be controlled by the etch time and other parameters of the formation of the porous material, as can the porosity, tortuosity, and other characteristics of the pores. The surrounding unetched material forms the vessel walls in this embodiment.

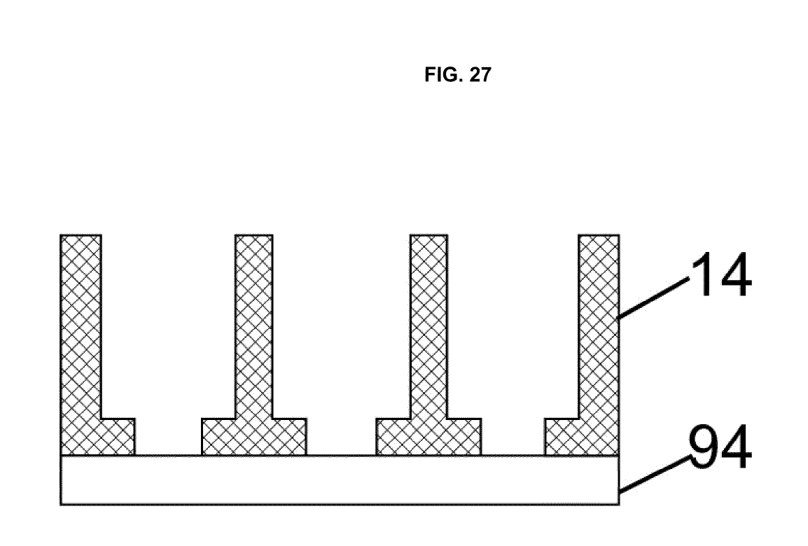

In another embodiment, the nucleic acid binding surface may be added to the vessel. For example it may be placed into the interior of vessel 14 (FIG. 25) or onto bottom of through-hole type vessel (FIG. 27). Nucleic acid-binding surfaces may be made of a variety of materials. In an embodiment, the binding surface is a glass fiber matrix. The glass fiber matrix may consist of layers of oriented fibers. In another embodiment, nucleic-acid binding surface may comprise silica beads or magnetic beads. In another embodiment, nucleic-acid binding surface may be high surface area polycarbonate. In another embodiment, the binding surface may be carbon fiber or activated charcoal. In another embodiment, the binding surface may be cellulose fiber. In another embodiment, any binding surface can be used which has a plurality of covalently linked oligo-dT, short random-complementary RNA sequences, and even specific RNA sequences. Other materials known to the field are provided in U.S. Pat. No. 7,229,595 which is hereby incorporated by reference in its entirety.

In one example, the binding surface is treated to improve the binding of nucleic acids. For example, the surface is functionalized with RNA hybridization molecules. In another example, the surface is treated with a high concentration salt, such as sodium iodide at 8 M.

In another embodiment, a nucleic acid binding surface was provided that did not autofluoresce at certain wavelengths of light to enable subsequent fluorescent detection of samples in a single vessel. This may be done by using black polycarbonate or by using silica filters with layers of oriented glass fibers, such as found in the filters of the PicoPure kit.

ii. Methods for Adding a Tissue Sample into the Vessel. A biological sample, such as a tissue sample, may be added into the vessel using a variety of methods. Tissue scrapes can be placed into the vessel. Tissue may be pushed into the vessel using the application of pressure to a tissue placed over the vessel, as illustrated in FIG. 3. The tissue may be covered by a gel solution, the gel may be solidified, and the tissue pulled into the wells by drying the gel. The tissue may be cut into small sections that are individually dropped into an array of vessels by a robot. The tissue may be microdissected by a laser onto a sealing film, and this sealing film used to seal the vessel plate. Other methods known to those in the art may be used.

Prior to adding the tissue, it may be treated in some manner For example, it may be homogenized or lysed. The tissue may therefore be pipetted into the vessel in solution form. Additionally, to provide controls, other tissues, an absence of tissues, purified RNA, and a dilution series of reagents known to those in the art may be provided in some of the wells of an array format.

iii. Methods for Adding a Protein Denaturing Agent. The denaturing agent may be added before, after, or together with the biological sample, such as a tissue sample. It can be added in a mixture with a lysis reagent containing surfactants, as is known in the art. It may be added by pipette or by filling due to capillary action, or by other methods known to those in the art. Pipetting can be performed manually or by robot. The denaturing agent may comprise guanidinium thiocyanate, guanidinium isothiocyanate or other guanidinium salts. Other denaturing agents known to those in the art include a mixture of guanidine isothiocyanate and phenol, a mixture of lithium chloride, Tris buffer, lithium dodecyl sulfate, Tween 20, and dithiothreitol, or other high or low concentrations of salts, or solutions of salts and detergents. Denaturants include HCl, urea, Trizol, lithium perchlorate, sodium Dodecyl sulfate (SLS or SDS). Denaturants may also include reagents that can disrupt specific bond linkages between amino acids in proteins, and can include dithiothreitol, 2-mercaptoethanol, and proteinase K.

In addition to the denaturing agent, a cell lysing agent or RNase inhibitor can be added. The cell lysing agent may be proteinase K, or it may be a detergent, such as Triton X-100 or Tween 20, or it may be phenol. As known to those in the art, there are many RNase enzymes that have specific inhibition towards a species, type of tissue, and type of RNase. The RNase inhibitor may comprise recombinant human placental RNase inhibitor, which inhibits human RNases A, B, and C. Ambion SUPERase-In inhibits RNases T1 and 1.

iv. Methods for Allowing Sufficient Time to Elapse to Free the Nucleic Acids. The denaturing and cell lysis or digestion reactions free the nucleic acids from the biological sample (such as tissue) and inactivate RNases by denaturing them, thereby preserving the RNA from degradation. The biological sample may be substantially dissolved in this step. Heating the mixture can speed up or improve the yield of the reactions. For example, the contents of the vessels may be heated to 45° C. for 30 minutes to give a good yield of RNA from the tissue. For tissue that is formalin fixed and paraffin embedded, treatment may take up to a week.

To minimize evaporation and/or cross-contamination, the reaction may be carried out in an environment containing a saturated vapor pressure of the same solvent(s) as is (are) in the vessel during the reaction. Another method to minimize evaporation and/or cross-contamination is by adding a material to the vessel that slows down the rate of water evaporation, such as BSA, glycerol, or agarose gel.

At the end of this step, the nucleic acids are dissolved or suspended in the solution. In particular, the more aggressive lysis reagents which include high concentrations of guanidinium salts, enable the nucleic acids to be preserved indefinitely at this stage.

v. Methods for Homogenization of Cell Lysate from the Sample. In some examples, a sample is homogenized during cell lysis. One method of homogenization is to allow sufficient time for the freed nucleic acids to diffuse or migrate throughout the lysate. As an alternative or as an additional step, heating, mixing, rolling, and/or general agitation can be applied to accelerate the homogenization. Yet another alternative is to accelerate the homogenization by including a magnetic component to the cell lysate, and mixing it by applying an alternative external magnetic field. Furthermore, the need for homogenization may be mitigated by providing a sufficiently thin section of tissue.

vi. Methods for Sealing the Vessel. In an embodiment of the method, the vessel is sealed to minimize evaporation of solvents and/or cross-contamination of adjacent vessels during reaction steps. Sealing films may include a solid backing of fluoropolymers, polyethylene, polypropylene, polyester, and silicone, which have good strength, flexibility, and resistance to gas or water vapor permeation. They also include an adhesive resin, e.g., made of acrylic resin or silicone resin, so that they exhibit properties such as resistance to degradation by water and solvents such as ethanol, strong adhesion, reversible sealing, heat activation, heat inactivation, light activation, or pressure activation. They may also include solid support films, such as glass slides, coated with a deformable coating, such as varnish or nail polish, or they may include bodies coated with a thin thermoplastic adhesive. They may include silicone or EDPM rubber. The seal need not be solid, but may comprise mineral oil. The seal may be pliable, such as wax (one example is the Chill-out liquid sealing wax sold by Bio-Rad, which is solid below 10° C. and molten above 20° C.). Any of the methods for sealing that are discussed herein or known to those of skill in the art can be used.

vii. Methods to Reduce Reagent Evaporation and Cross-Talk Using Seals. The same methods may be used as appropriate for sealing the vessel. To aid the removal of a reversible seal, and/or to minimize cross-contamination of adjacent vessels, the fluid can be frozen in the substrate or vessel. This can prevent the contents of the vial from being partially or completely removed together with the seal. In another embodiment, the tissue lysates from previous steps may be encapsulated by gel, such as agarose, to prevent the contents of the vials from being removed with the seal.

viii. Methods for Adding a Nucleic Acid Precipitation Agent to the Vessel. The nucleic acid precipitation agent causes the nucleic acids to leave the aqueous phase and adhere to the nucleic acid binding surface. The precipitant may be a high concentration of salt, such as 0.5 molar strength lithium chloride, or 6 molar strength guanidinium isothiocyanate. The precipitant may also be a solvent such as ethanol, methanol, acetone, n-butanol, or isopropanol. When the salt concentration is high, the addition of solvent is believed to enhance the binding efficiency of nucleic acids. When the salt concentration is low, particularly below about 0.1 to 0.2 molar, the solvent must be used to maintain nucleic acids on the binding surface. The precipitation allows the other contents of the vessel to be selectively removed, separating them from the nucleic acids and thereby purifying the nucleic acids. At the end of this step, the nucleic acids are reversibly bound to the binding surface.

Ethanol or ethanol-containing solutions may be used to effect the precipitation. Other solvents in which the nucleic acids are insoluble can also be used.

ix. Methods for Combining Processing Steps. In an embodiment, nucleic acids are simultaneously released from the biological sample, such as tissue, and captured onto a binding surface, such as to increase the simplicity of the system. For example, the cell lysis, RNase inactivation, and lysate homogenization step are performed at the same time as the RNA capture step, by providing the biological sample into a vessel that already contains the nucleic acid binding surface (or vice versa) and adding a mixture containing cell lysis reagent(s), protein denaturant(s), and nucleic acid precipitant(s) at the same time (or adding the reagents to a vessel that already contains the biological sample and the binding surface, or using another order for the addition of binding surface, tissue, and reagents).

x. Methods for Degrading DNA or RNA. In some embodiments, it may be desired to purify the nucleic acids further, for example to have only RNA or only DNA. For example, DNA from a tissue is of comparable quantity to that of the cytoplastmic total RNA (6.6 pg DNA versus 10 to 15 pg RNA per cell), and may thus reduce the efficiency of a subsequent PCR reaction, or create false positives, and/or artificially increase the apparent yield of RNA made by a spectroscopic measurement. This may be accomplished by degrading the DNA or RNA, respectively. To degrade the DNA, DNase can be used, and to degrade the RNA, RNase can be used. The small fragments that result from the reaction with DNase or RNase may be washed off the binding surface. For example, DNase is mixed with a buffer and water and added to the vessel. The solution is allowed to react at room temperature for 5-10 minutes. Thereafter, the remainder of the vessel is filled with an ethanol solution and the fluid is subsequently removed. In another embodiment, DNA can be partially degraded by shearing by vigorous flow through a filter matrix, to reduce its size from hundreds of thousands of base pairs to thousands of base pairs, as known to those in art.

xi. Methods for Removing Unbound Species from the Vessel. In an embodiment, the nucleic acids are separated from the rest of the tissue components and reagents, completing the purification, by removing the fluid from the vessel. This may be done by pouring off the fluid, draining the fluid, applying suction or pressure to aid the draining of the fluid, blotting, centrifugation, and other methods known to those in the art.

The purification may be improved by following fluid removal by rinsing. This step may comprise multiple rinses. The composition of the rinse solutions may be changed to increase the concentration of non-solvent for nucleic acids; for example, the concentration of ethanol in a water/ethanol solution may be increased. The rinsing is accomplished by filling the vessel with the rinse fluid, and then removing the rinse fluid, methods for which have been described above.

xii. Methods for Adding a Blocking Agent to the Vessel. To 1) prevent the adsorption of other species on the binding surface and/or 2) to free the nucleic acids from the binding surface, a substance is added to the vessel that binds to the binding surface more strongly than the nucleic acids bind to the binding surface. In the first case, this facilitates replication reactions, such as reverse transcription of RNA and PCR, by preventing the adsorption onto the binding surface of species that are needed for the reaction. In the second case, the nucleic acids are thereby displaced, and placed into the solution phase. The fluid should be a solvent for the nucleic acids. The presence of non-solvents such as ethanol may interfere with the release.

In one example, BSA is used as a blocking agent. Example concentrations are 0.01% to 20% weight per volume in water. Other blocking agents include Tween 20, up to about 0.5% w/v, and Triton X-100 up to about 5% w/v 0.75% poly ethylene glycol 8000, 1% poly vinyl pyrolyidine 40000. Proteins, such as casein can and/or other nucleic acids, such as salmon sperm DNA, can be used. Hybridization of similar DNA may also be efficacious.

In another embodiment, complementary strands of the nucleic acids are transcribed using reverse transcriptase or polymerases, and then annealed to free a copy of the captured nucleic acids from the binding surface for subsequent detection, as known to those in the art.

At the end of this step, the nucleic acids in the blocking solution are ready for subsequent manipulation, analysis, amplification, or detection. This can occur without further preparation and in the same vessel.

### Example 16

### Methods to Enable Downstream PCR Amplification After Nucleic Acid Isolation

This example describes studies performed to determine which components inhibit or enable PCR amplification in a single vessel directly from a standard silica-filter based nucleic acid extraction and purification method.

i. Concentration of PicoPure Reagents in the PCR SuperMix. Studies were performed to identify the approximate percentage, if any, of the components contained in the PicoPure isolation kit that would affect a PCR reaction. The reagents in the kit included elution buffer (EB), conditioning buffer (CB), wash buffer 1 (WB1), wash buffer 2 (WB2), and the silica binding filter (filter). These reagents were diluted, or cut into pieces in the case of the filter, placed into standard thin-walled PCR tubes, and dried at 105° C. Standard PCR SuperMix (Thermo-Scientific AB-0301-a) was added containing control genomic DNA to 395 copies per tube, a GAPDH detection primer/fluorescent probe set, and MgCl₂ to 3 mM. These tubes were then subjected to PCR thermocycling (95° C. for 5 seconds, 60° C. for 25 seconds, and 50 cycles) and imaged on a Typhoon 9410 imager with settings of 488 nm excitation and 500 pmt detection intensity. By comparing the fluorescent intensity of positive controls with PicoPure reagents to standard controls, PCR inhibition could be ascertained.

TABLE III
Results of testing various PicoPure kit components during PCR.

| Tube # | PicoPure Component Tested | Inhibition Result |
|---|---|---|
| 1 | 100% Elution Buffer | None |
| 2 | 20% Elution Buffer | None |
| 3 | 5% Elution Buffer | None |
| 4 | 2% Elution Buffer | None |
| 5 | 10% Conditioning Buffer | Complete |
| 6 | 1% Conditioning Buffer | None |
| 7 | 10% Wash Buffer 1 | Partial |
| 8 | 1% Wash Buffer 1 | None |
| 9 | 10% Wash Buffer 2 | None |
| 10 | 1% Wash Buffer 2 | None |
| 11 | 90% of a PicoPure Silica Filter* | Complete |
| 12 | 10% of a PicoPure Silica Filter* | Complete |

*Percentage of the size of the filter included in the PicoPure kit.

The results (Table III) showed that PCR is inhibited by inclusion of any silica binding filter, 10% conditioning buffer, or 10% wash buffer 1. Since the PicoPure procedure uses subsequent washes using wash buffer 2, which did not inhibit PCR, it is expected that any trace of conditioning buffer or wash buffer 1, which come first, would be eliminated by wash buffer 2, and that the use of conditioning buffer or wash buffer would thus not inhibit PCR. Therefore, the major inhibitor of PCR is the silica binding filter, which is designed to capture nucleic acids. The reason for the inhibition may be that the filter bound the DNA template and/or the DNA polymerase.

ii. Mitigation of PicoPure silica filter inhibition of PCR. Studies were performed to identify if there were any reagents that could be added to the PCR solution to reverse the inhibition caused by the silica filter. The reagents tested were elution buffer, bovine serum albumin (BSA), DNA polymerase, and nothing (control). These three reagents were added to two groups of tubes containing 10% by weight of filters that were either pre-treated with conditioning buffer or left untreated, to see if the pretreatment would have any effect. The same protocol as described above was used for the addition of reagents, PCR SuperMix, thermocycling conditions, and imaging to ascertain PCR success or failure.

TABLE IV
Results of testing reagents for mitigation of
PCR inhibition by the silica filter.

| | | Filter | Inhibition |
|---|---|---|---|
| ube # | Mitigation component | Pretreatment | Improvement? |
| | None | Yes | No |
| | 100% Elution Buffer | Yes | No |
| | 0.1% BSA | Yes | Partial |
| | 150 U/mL Polymerase | Yes | Complete |
| | None | No | No |
| | 100% Elution Buffer | No | No |
| | 0.1% BSA | No | Complete |
| | 150 U/mL Polymerase | No | No |

The results (Table IV) showed that either 1) filter pretreatment and additional DNA polymerase, or 2) no filter pretreatment and addition of BSA could completely reverse PCR inhibition. The BSA more consistently reversed PCR inhibition, for either filters that were pretreated or not. The results suggest that PCR was inhibited by the BSA blocking the silica filter and releasing molecules bound to the surface, possibly both DNA polymerase and DNA template. Furthermore, Table IV suggests that the elution buffer itself does not prevent nucleic acids from adhering to the silica filter, since PCR in the presence of the elution buffer and the filter is inhibited.

### Example 17

### Methods to Simplify Nucleic Acid Isolation from Tissues: Combination of Nucleic Acid Lysis, Preservation, Precipitation, and Homogenization

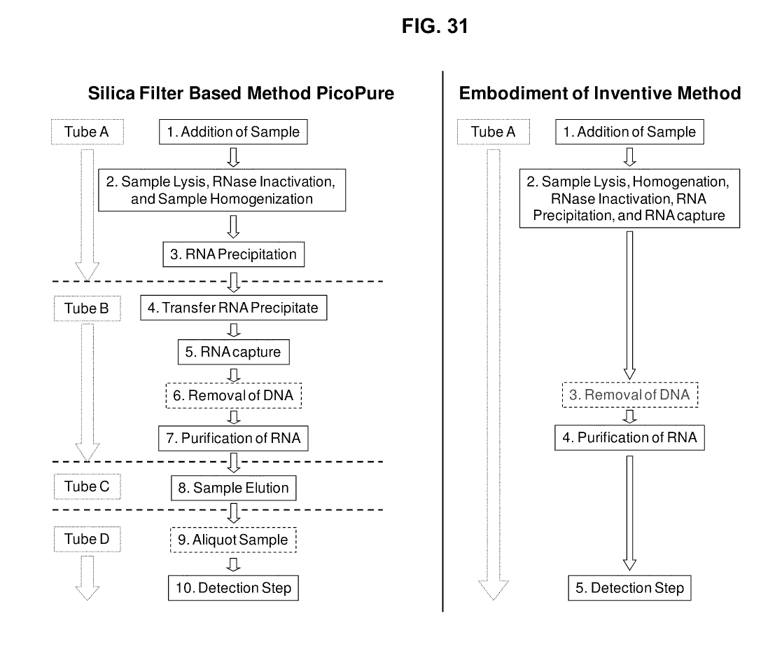

The standard PicoPure procedure was used in this example. Extraction Buffer, to both lyse cells and preserve the RNA by denaturing native RNases, simplifying these two steps into one process (see FIG. 31). However, there are three subsequent steps in the standard protocol that increase the complexity and handling required for RNA isolation. First, it is suggested that 70% ethanol be added to the lysates to precipitate nucleic acids from the solution phase. Second, it is suggested that the lysates be re-pipetted to homogenize the mixture. Third, it is suggested that the lysates be pipetted onto a silica filter to bind the precipitated nucleic acids. These steps require two tubes and direct user handling of the samples.

These three steps were modified in several variations, on both xylene-fixed and frozen normal breast tissue specimens, to develop a simplified protocol for nucleic acid purification from tissues. First, the step of pipetting reagents to the filter was combined with the extraction step. Second, the step of extraction was combined with the step of adding a precipitant. Third, as negative controls, these procedures were tested without silica binding filters. In each of these tests, the re-pipetting step was eliminated. Tissue sections from 4 mm² to 6 mm² in area and 8 μm thick were used for each tube. Finally, as positive controls, the procedure was carried out as specified in the PicoPure manual. The quantity of nucleic acids collected by each procedure was measured using a Nanodrop Spectroscopic Meter, set to measure the RNA-40 spectrum, to determine the efficiency of extraction. Areas of 4 mm² to 6 mm² of 8 μm thick tissue sections were used for each tube.

TABLE V
Results of testing combinations of the existing PicoPure procedure.

| Tube | | Components | Components | Nucleic Acid |
|---|---|---|---|---|
| # | Tissue Type | Combined | Eliminated | Quantification |
| 1 | Frozen Normal Breast | None | None | 4.0 ng/μL |
| 2 | Xylene Fixed Normal Breast | None | None | 5.5 ng/μL |
| 3 | Frozen Normal Breast | None | None | 6.7 ng/μL |
| 4 | Xylene Fixed Normal Breast | None | None | 5.1 ng/μL |
| 5 | Frozen Normal Breast | Extraction and | Re-pipetting for | 4.2 ng/μL |
| | | pipetting onto Filter | homogenization | |
| 6 | Xylene Fixed Normal Breast | Extraction and | Re-pipetting for | 5.5 ng/μL |
| | | pipetting onto Filter | homogenization | |
| 7 | Frozen Normal Breast | Extraction and | Re-pipetting for | 3.7 ng/μL |
| | | precipitation | homogenization | |
| 8 | Xylene Fixed Normal Breast | Extraction and | Re-pipetting for | 4.0 ng/μL |
| | | precipitation | homogenization | |
| 9 | Frozen Normal Breast | None | Binding Filter | 0.8 ng/μL |
| 10 | Xylene Fixed Normal Breast | None | Binding Filter | — |
| 11 | Frozen Normal Breast | Extraction and | Binding Filter | 0.5 ng/μL |
| | | precipitation | | |
| 12 | Xylene Fixed Normal Breast | Extraction and | Binding Filter | 1.2 ng/μL |
| | | precipitation | | |

The results in Table V show that for the four positive control runs, an average of 5.4 ng/μL of nucleic acids were extracted. The results for combining the extraction and pipetting-onto-filter steps showed an average of 4.9 ng/μL of nucleic acids. The results for combining the extraction and precipitation steps showed an average of 3.9 ng/μL of nucleic acids. The results for the negative controls showed an average of 0.8 ng/μL of nucleic acids.

Because the initial range of tissue quantity used varied by 50%, there was no significant difference in quantity of nucleic acids isolated between positive controls and tubes in which the components of the PicoPure protocol were combined and/or eliminated. Therefore, it can be concluded that the silica-filter based method of extracting nucleic acids from tissue can be substantially simplified by performing extraction and precipitation in a single tube, and by eliminating re-pipetting for sample homogenization.

### Example 18

### Alternative Filter Materials

This example describes the effect of filter composition on nucleic acid purification efficiency.

To determine if other materials can bind nucleic acids efficiently, several materials were tested in place of the standard silica binding filter. A modified PicoPure procedure was used with a simulated tissue lysate consisting of control mRNA. The mRNA was diluted in a 1:1 mixture of extraction buffer and 70% ethanol, and the PicoPure silica filter was replaced with the alternative binding surface. The eluted nucleic acids were again quantified using the Nanodrop spectroscopic meter as before, and the results were compared and normalized against the standard, unmodified PicoPure materials and procedure to determine purification efficiency.

The binding surfaces tested were the standard PicoPure filter as a positive control, an activated carbon-coated air filter, Whatman F/B glass fiber filter paper, a Qiagen Miniprep filter, carbon fiber threads, polyester filter sheet, black polycarbonate membrane, brass sheet grinds, carbon fiber sheet grinds, and polycarbonate sheet grinds. All filters were pretreated with conditioning buffer as specified in the PicoPure protocol. The results are shown in Table VI.

It was also determined, in a separate set of studies, whether those filters that successfully extracted RNA could be used directly for TaqMan-based fluorescent PCR detection. Filters were pretreated with conditioning buffer, cut into pieces of 6 mm² area, and placed into standard PCR tubes. Standard one step RT-PCR Verso SuperMix was added along with 0.2% BSA in the SuperMix to prevent inhibition of PCR. The samples were thermocycled (50° C. for 10 minutes, then 95° C. for 5 seconds, 56° C. for 5 seconds, 72° C. for 10 seconds repeated for 40 cycles). The success of PCR was determined by placing the tubes in a Typhoon imager and looking for fluorescence as previously described. These results are also shown in Table VI.

TABLE VI
Results of testing different filter materials in place
of the PicoPure filter (normalized to tube 1).

| | | | | Effect on |
|---|---|---|---|---|
| | | Nucleic Acids | | TaqMan |
| Tube | | Quantification | | Fluorescent |
| # | Filter Material | (ng/tube) | Efficiency | Detection |
| 1 | PicoPure | 1060 | 100% | No effect |
| | | | (Baseline) | |
| 2 | Carbon air filter | 201 | 19% | Signal |
| | | | | attenuation |
| 3 | Whatman F/B | 1043 | 98% | Background |
| | | | | signal |
| | | | | amplification |
| 4 | Whatman F/B | 1043 | 98% | Background |
| | | | | signal |
| | | | | amplification |
| 5 | Carbon fiber | 622 | 59% | Minor |
| | | | | background |
| | | | | signal |
| | | | | amplification |
| 6 | Polyester filter | 126 | 12% | N/A |
| 7 | Black polycarbonate | 312 | 29% | No effect |
| | 0.6 μm pores | | | |
| 8 | Brass sheet grinds | 16 | 1.5% | N/A |
| 9 | Carbon fiber | 36 | 3.4% | N/A |
| | sheet grinds | | | |
| 10 | Polycarbonate | 105 | 10% | N/A |
| | sheet grinds | | | |

The results in Table VI show that all of the filters were capable of binding and purifying some amount of nucleic acid, and that the filters were not destroyed by the procedure used. The silica filters were shown to have the improved binding efficiency, followed by the carbon filter and the black polycarbonate membrane. However, it should be noted that while the thickness of most of the filters tested was comparable, the black polycarbonate membranes had a thickness of only 10 μm, while the PicoPure filter was about 1500 μm thick. Given that the efficiency of the polycarbonate was 29%, these membranes may have the potential for higher efficiency extraction if stacked to the same thickness as the silica membranes.

Of the filters tested in TaqMan fluorescent one-step RT-PCR, only the polycarbonate and PicoPure filters did not either amplify or attenuate the signals. The carbon filters produced false negatives and false positives, and the Whatman filter produced false positive as well. One possible explanation is that this is due to particular residue from the filters, thus in an embodiment the filter is prewashed to reduce particulates and in another embodiment the filter is placed onto a secondary filter which does not capture nucleic acids by prevents the interference of particular matter. Thus it can be concluded that for direct one-step RT-PCR amplification of RNA bound to a filter, one choice is PicoPure silica filters followed by the black polycarbonate membranes.

### Example 19

### Demonstration of Tissue Extraction, Purification, and Detection in a Single Vessel

This example demonstrates tissue lysis, RNA extraction, preservation, and precipitation in a single vessel, and subsequent purification and detection in a single vessel format, performed on a multi-well plate. During the following experiments, RNA lysate from the same initial vessel was used to ensure that the RNA content would be the same across all wells of the multi-well plate.

i. Detection Efficacy from Tissue Lysates Compared with Control mRNA in Wells of Two Geometries. It was tested whether mRNA can be purified from tissue and detected directly in wells of two different geometries that were drilled into a polycarbonate plate to form a multi-well plate. The two styles of well are illustrated in FIGS. 25 and 26. For the first, the aperture at the bottom of the well was drilled into the polycarbonate; this type 1. In the second, the apertures were formed in a sealing tape adhered to the bottom of through-hole style wells; this is type 2. The wells had dimensions of 3.125 mm diameter and 3.125 mm depth, and the aperture was 1 mm in diameter. A PicoPure filter of size 2×2 mm was added to every well.

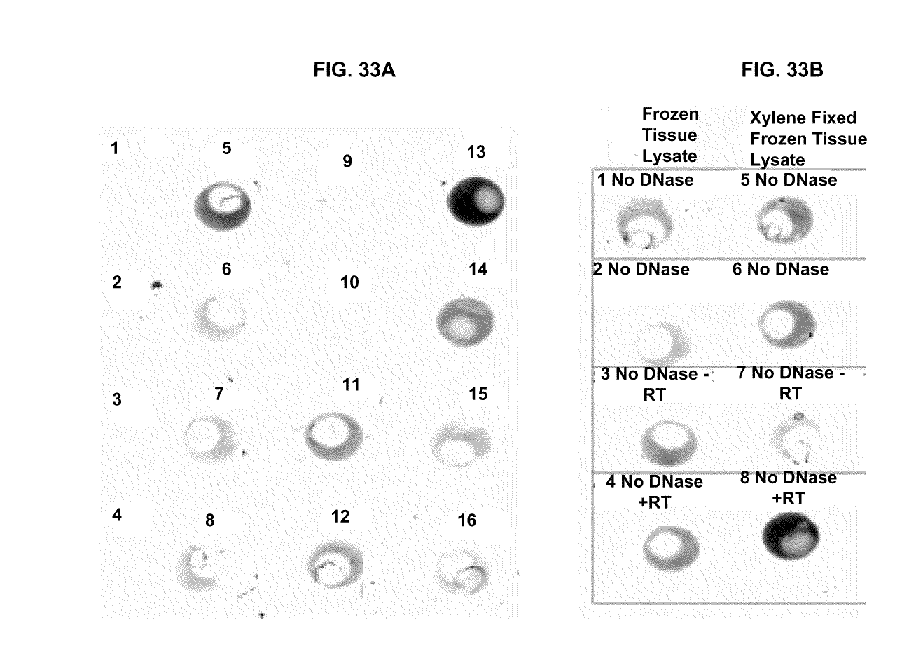

First, an RNA dilution series (3 ng, 300 pg, 30 pg, 0 pg) was placed into four wells of type 1. To determine the effects of vessel geometry on signal intensity, the same RNA dilution series was also placed into four wells of type 1. 13 μL of xylene-fixed and frozen normal breast tissue lysates were pipetted into a fifth and sixth well of type 1. This fluid was drained from the wells using vacuum suction approximately 1 minute later. The nucleic acids were expected to have bound to the filter. Each of the two sample wells that had been loaded with lysate were washed once with 13 μL of Wash Buffer 1 and twice with 13 μL of Wash Buffer 2. Vacuum suction was used between wash steps to remove the fluid from the bottom of the wells. The substrate was heated to 85° C. for 3 minutes to ensure evaporation of solvents that could inhibit PCR. Then, 13 μL of one-step RT-PCR Verso SuperMix containing 2% by volume reverse transcriptase (RT) and 0.4% BSA was added to the wells of type 1, and 17 μL was added to the wells of type 2. All the wells in the plate were sealed, thermocycled, and imaged on the Typhoon imager as previously described. The results are shown in FIG. 33A.

The results in FIG. 33A showed that nucleic acids from tissue (wells 11 and 12) were amplified with sufficient intensity to detect. These levels were just below the level of 3 ng of control RNA in similar wells (5-8); so RNA amplification was conclusively demonstrated. This study also demonstrated that the same volume of wash buffers and PCR SuperMix could be used: whereas the standard protocol recommends hundreds of micro-liters of wash buffers, only 13 μL were used in these experiments. The studies showed that to achieve higher signals, the wells should be of type 2, as seen in wells 13-16, which are much brighter.

ii. Verification of the Presence of mRNA in the Tissue Sample. To further validate that positive fluorescent signals from the wells with lysate originated from mRNA, rather than being false positive signals, several control samples were tested on a new plate with wells of type 1. To provide a baseline, duplicate samples of the frozen and xylene-fixed tissue lysates were tested for detection as before. The lysates were also tested with DNase treatment and with and without reverse-transcriptase (RT) to determine if any interfering DNA was detected. The substrate was processed as above for wells of type 1. The results are shown in FIG. 33B.

The results in FIG. 33B show that to achieve the higher signals, DNase treatment may be used before PCR, as seen by well 8. Furthermore, the highest signals came from tissue treated with xylene, also seen by well 8. The xylene may prevent the degradation of RNA when tissues reach room temperature. The experiment also showed that without reverse transcriptase, no signal was detected. This result, in combination with the signal obtained using RT, shows that mRNA was present in the sample and that it was detected using the disclosed protocol.

### Example 20

### Methods for Ensuring Consistency and Efficiency Between Different Studies

This example summarizes the different approaches used to ensure maximum consistency and efficiency when either extracting, purifying, or detecting mRNA from a sample or detecting mRNA directly bound to a filter.

i. Validation of mRNA Amplification. It is beneficial to verify that the fluorescent TaqMan signal for positive controls corresponds to the desired mRNA target amplified by PCR, as determined by agarose gel electrophoresis. In one experiment, wells were filled with 3 ng of control RNA (positive) or with no RNA. They were processed as described above. Samples of 2.4 μL were diluted 1:4 in a gel loading dye and run on a 2% NuSieve gel in 1× TAE buffer at 100 V for 30 minutes. The result was imaged on the Typhoon imager.

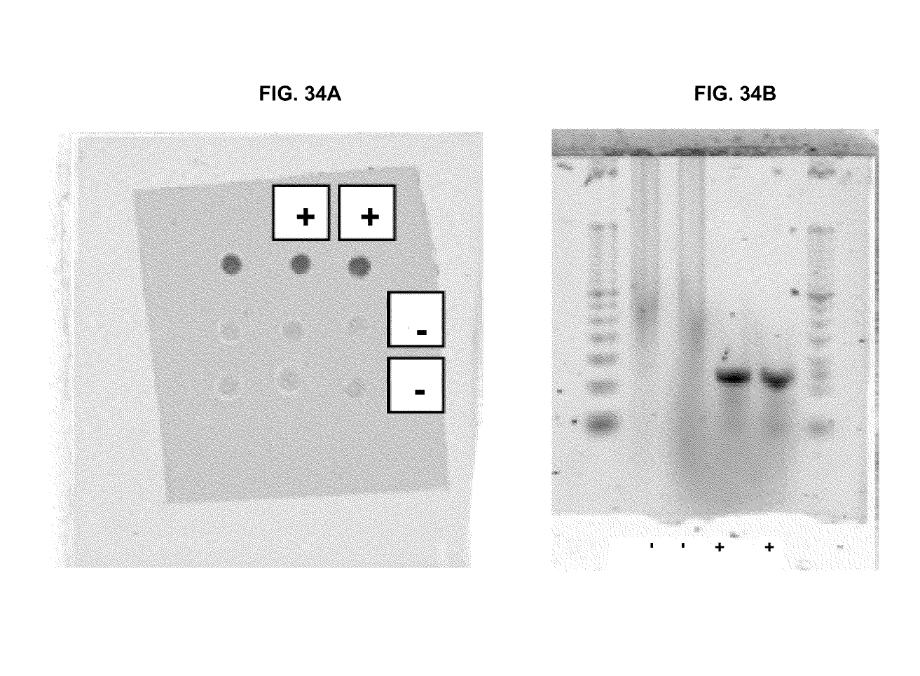

The results in FIG. 34A showed a clear positive signal (dark spots) from the TaqMan fluorescent assay when mRNA had been pipetted into the wells Negative samples had no signal. The results in FIG. 34B showed that the positive fluorescent wells corresponded with a 120 nt GAPDH mRNA amplicon, proving a signal-target relationship. Once the signal correlation with the target amplicon has been established as accurate, it is known to those in the art that a positive TaqMan signal very reliably indicates the amplification of the desired target mRNA, and that no further validation is needed.

ii. Need to Remove DNA from Tissue Lysates. It should be noted that removal of DNase substantially improves the signal intensity and efficiency of amplifying mRNA. These results were previously described in FIG. 33. The reason for this efficiency improvement may be due to mispriming of the primers, probes, or polyermase to non-specific targets.

iii. Kit Age and Centrifuge Speed or Vacuum. Studies have shown that with older kits,

RNA quality may degrade significantly. This was determined using an Agilent Bioanalyzer on the same tissue samples using non-expired reagents. Furthermore, the solution of 70% ethanol with extraction buffer may need to be freshly mixed, such as on a weekly or monthly basis, to prevent RNA degradation.

iv. Quality and Type of BSA. Higher purity of BSA (e.g. 99% purity or greater) can improve results. Furthermore, BSA that is non-acetylated may also improve results. It is known to those in the art that impure BSA or acetylated BSA can inhibit PCR by interfering with the function of DNA polymerase.

v. Cleanliness of Substrate Surface. Cleaning can remove commercial residues, RNases, and residual nucleic acids. When using metals such as aluminum, washing with hot soapy water (using a commercial soap) results in inconsistent performance A new substrate may be created for each experiment. Condensation on surfaces may affect sealing films.

vi. Type of Filter and Filter Placement. When testing Whatman F/B filters, it was discovered that the loosely held together matrix sheds glass particles, which can interfere with downstream fluorescent measurements. Furthermore, the filters may need to be free from binders or manufacturing residues. In addition, if a vacuum is used to wash fluid during the purification step, a larger filter can help to prevent the filter from being pulled into the vacuum.

viii. Length of Capture Time. It should be noted that if the lysates containing precipitated nucleic acids are not held on the binding surface for a sufficient length of time, the nucleic acids in the sample may be washed away during the rinsing steps. During the initial binding step, nucleic acids were added with no wait time and 5 minutes of wait time before purification. The failure rate of the samples with no wait time was 75%, while those samples with 5 minute wait time had no failures.

### Example 21

### Single Vessel Methods for Dynabeads® mRNA Isolation

This example describes single vessel methods for Dynabeads® mRNA isolation.

The Dynabeads® mRNA isolation kit provides paramagnetic beads with oligo-dT moieties, which allows mRNA to be captured by hybridization (in under a minute) and pelleted by an external magnetic field to wash away undesired components. It is well known that the Dynabeads® mRNA kit can be scaled to low volumes to work with RNA from single cells. To date however, no protocol has been described for lysing tissue, capturing mRNA, and purifying the mRNA in a single well. This is because it is typical to centrifuge cellular artifacts to obtain a clear cellular lysate; the artifacts would otherwise inhibit the washing of beads.

To this end, a modified protocol has been developed that eliminates the need for two vessels by using a highly aggressive lysis buffer. This modification is in line with the goal of mapping mRNA from tissues by performing several single-well isolation procedures in a multi-well substrate. To achieve this goal, several more modifications were reduced to practice. Key steps were modified to eliminate re-pipetting steps recommended for the Dynabeads® mRNA isolation by using vortexing. The use of DNase treatment on mRNA captured by the Dynabeads® system was also tested. A Dynabeads® protocol was optimized for using PicoPure extraction buffer, and a new wash protocol was developed for using solvent.

i) Modified Protocol for Simplified/GITC Single-Vessel mRNA Isolation with Oligo-dT Magnetic Beads

The recommended oligo-dT mRNA isolation protocol was previously published (Jakobsen Nucleic Acids Research 18(12): 3669, 1990), and is identical to the present DynaBeads® mRNA direct user manual (as available on Jan. 23, 2010). By this protocol, tissue is mechanically fragmented using a mortar and pestle at liquid nitrogen temperatures. Tissue is then chemically lysed in buffer and simultaneously mechanical homogenized using a glass tube and Teflon pestle. The lysate is then centrifuged to remove cellular debris, and the clear lysate is placed in a second vessel. Magnetic oligo-dT beads are then added to this second vessel. Several wash steps are then needed to remove unbound species from the vessel.

In a study, when crude tissue lysate containing cellular residue was mixed directly with the beads, the tissue caused the beads to chimp, inhibiting subsequent washing steps and preventing subsequent mRNA detection.

ii) Optimization of GITC Buffers

To simplify the protocol, and to improve mRNA yields, experiments were performed to see if the aggressive GITC lysis/extraction buffer in the formats of the PicoPure mRNA isolation or Qiagen RNeasy kits could be used with DynaBeads to better lyse tissues in the same vessel used for the binding and purification. Although the DynaBeads® protocol claims that all known buffer systems are compatible with the beads, the manufacturer only references the use of the less-aggressive GITC buffer (containing 4 M GTC, 0.5% sarkosyl, 1% DTT, 0.5 M LiCl, 0.1 M Tris pH8), and uses a two-vessel protocol (Meijer et al., National Inst. Publ. Health and Env. Protection Bilthoven, Report 118504 001 The Netherlands, 1995).

In a study, a first set of vessels was used to test various extraction buffers and concentrations. The use of GITC extraction buffer to lyse, bind, and purify mRNA from frozen animal tissues was tested. These samples were compared against the Dynabeads® lysis buffer as a control. Tissue from mouse frozen liver was sectioned at 10 micron thickness onto glass. Mouse liver was chosen for its intermediate level of ribonucleases. Portions of tissue approximately 1 mm×5 mm were then placed in nine 500 mL vessels. The vessels initially contained the buffers listed in the following table, with some initial amount of water. Vessels were vortexed aggressively, heated to 50° C. for 30 minutes and 25° C. for 30 minutes, and then briefly centrifuged. Then 5 μL samples were taken and placed into new vessels. These vessels had 1 μL of water-washed oligo-dT beads added, and a final amount of water or Dynal buffer added to ensure that GITC buffer concentrations were at a maximum of 25% of the volume; this is because addition of unmodified Picopure or Qiagen lysis buffers prevents the detection of mRNA. Vessels were then washed using 20 μL of Dynal wash buffer B while holding vessels over a magnet to removing wash buffer. Instead of re-pipetting in wells to re-suspend beads, vortexing was used. Finally 10 μL of 10 mM tris was added to the vessels. 1 μL samples were pipetted into a qPCR plate and the target mRNA GYS2 was amplified for 35 cycles with verso one-step mix.

In a second set of vessels shown in the table below, different volumes of tissue lysed with 100% PicoPure extraction buffer as previously described were mixed with water to test the effect of salt concentration on mRNA binding efficiency. Volumes for tissue, beads, and buffer were doubled.

In a third set of vessels, liver was again isolated using 100% PicoPure extraction buffer. However, a fixed amount of this lysate (1 μL) was mixed with varying concentrations of PicoPure extraction buffer in water to test hybridization efficiency. These concentrations were 10%, 20%, 40%, 60%, 80%, and 100%.

| | Lysis Buffer | Initial | Final Addition w/1 μL |
|---|---|---|---|
| Vessel | (μL) | Water (μL) | Beads Each (μL) |
| 1 | 0 | 20 | 0 |
| 2 | 1 | 19 | 0 water |
| 3 | 2 | 18 | 5 water |
| 4 | 5 | 15 | 20 water |
| 5 | 10 | 10 | 45 water |
| 6 | 20 | 0 | 95 water |
| 7 | (Dynal buffer) 20 | 0 | 0 |
| 8 | (RLT buffer) 20 | 0 | 15 water |
| 9 | (RLT buffer) 5 | 15 | 15 Dynal buffer |

| | Picopure | Initial | Estimated Salt |
|---|---|---|---|
| Vessel | Lysate | Water | Concentration (mM) |
| 10 | 0 | 38 | 0 |
| 11 | 0.5 | 37.5 | 50 |
| 12 | 1 | 37 | 100 |
| 13 | 1.5 | 36.5 | 150 |
| 14 | 2 | 36 | 200 |
| 15 | 2.5 | 35.5 | 250 |
| 16 | 3 | 35 | 300 |
| 17 | 4 | 34 | 400 |
| 18 | 5 | 33 | 500 |

The results showed that mRNA could not be isolated using the buffer RLT from the Qiagen Kit, which is likely due to an unknown chemical incompatibility between the kits. The results showed that PicoPure extraction buffer could be used, as long as the initial concentration of buffer was between 50%-100% PicoPure extraction buffer. The results showed that below a level of 5%, PicoPure extraction buffer in water (an estimated 200 mM salt concentration, based on an MSDS from the company), mRNA did not bind efficiency and most of it was lost; above a concentration of 40% PicoPure extraction buffer, most of the mRNA was also lost (an estimated 1.6 M salt concentration). Optimal lysis conditions were 100% PicoPure lysis buffer, and optimal mRNA hybridization levels were between 5% and 40% PicoPure lysis buffer.

These results showed that mRNA can be lysed with GITC buffer from the PicoPure mRNA extraction kit, and captured and purified using Dynal Oligo-dT magnetic beads. This shows that the Dynal kit is not compatible with PicoPure without modifications. The present studies, however, showed that the two kits can be made compatible if an initial lysis is performed in 100% PicoPure extraction buffer, and then diluted to a level between 5% to 40% PicoPure extraction buffer in water to permit hybridization. Furthermore, these results showed detection of targets at about 1 to 2 fewer PCR cycles, showing that this new method performs better than the previous one, and it is also known that this buffer is better for use on tissues with high concentrations of ribonuclease. Negative controls were unremarkable.

iii) Example of a Single-Vessel PicoPure-Dynabeads Protocol, and Evidence of Greater Yields

Further studies were conducted to see if yields of mRNA were greater when using the new PicoPure-Dynabeads protocol compared with the manufacturer's protocol. Four vessels containing 40 μL of Dynabeads® lysis buffer and four vessels with 10 μL PicoPure extraction buffer were created. Mouse frozen brain was tested, to ensure that there was no bias against the Dynabeads® protocol by RNases. The tissues were sectioned to 10 micron thickness and placed on several glass slides. For each brain, a randomly chosen half was scraped into a PicoPure vessel, and the other half into a Dynabeads® vessel, to ensure equal amounts of tissue. Vessels were aggressively vortexed and left to lyse for 30 minutes. Vessels with PicoPure buffer then had 30 μL of water added to lower the concentration of salts to promote hybridization. 8 μL of water-washed oligo-dT beads were then added to all vessels, and the vessels were then vortexed and allowed to hybridize for 5 minutes. Vessels were washed on a magnet using two 50 μL washes of Dynabeads® mRNA direct kit Wash A, and two washes of Wash B Finally, 20 μL of 10 mM Tris was added to the vessels. During all wash steps, beads were re-suspended by vortexing. 1 μL samples from each vessel were pipetted into a qPCR plate, in triplicate, and HPRT mRNA was amplified using the AgPath-ID kit as specified in Example 3.

The results consistently showed a 4-fold increase in the detection of HPRT mRNA (two earlier PCR cycles). This experiment shows that it is possible to lyse, capture, and purify mRNA all in a single vessel, using our new protocol. Furthermore, using a protocol to enable PicoPure lysis buffer dramatically increased the yield of captured and detected mRNA. The experiment suggests that PicoPure extraction buffer improves the yield of mRNA by lysing tissues more completely, because the mRNA capture efficiency is expected to be well over 50% using the Dynabeads® system.

iv) Use of Solvents to Wash Nucleic Acids

In an example, several solvents were tested for their ability to preserve mRNA, to keep mRNA bound to oligo-dT magnetic beads during a wash, and for their general compatibility with the Dynabeads® system. The solvents tested were 2-propanol, n-butanol, methanol, isopropanol, and acetone.

To see if the solvents can damage RNA, a 10 μL mixture was prepared containing each of these solvents at 99% concentration in water, and control human RNA totaling 3 ng. The solutions were heated to 50° C. for 10 minutes to evaporate the solvents.

To see if the solvents can strip RNA bound to beads, 6 vessels were prepared containing 3.2 μL of Dynabeads® lysis buffer with 0.8 μL of lysis-buffer-rinsed Dynabeads, and 1 μL of 30 ng/control human RNA. The RNA was hybridized for 5 minutes and then washed twice using 50 μL of wash A and then 50 μL of wash B. A second wash B was performed for a control vessel, while for all other vessels, 50 μL of each solvent was applied. Vessels were then vortexed, and 1 μL samples were placed in a qPCR plate and dried down at 50° C. for 5 minutes. PCR was then performed using a 10 μL reaction volume, with Verso one-step PCR mix containing the Bactin6 probe set.

The results showed detection of mRNA when mRNA was incubated with various solvents. However, the detection levels of mRNA when incubated with n-butanol dropped by a factor of about 10. This indicates that mRNA is easily damaged by most solvents. When beads with bound mRNA were washed with solvents, mRNA levels were reduced by about 75%. By extension, it is likely that mRNA can be hybridized in these solvents as well. Various embodiments of the invention can use solvents such as ethanol, acetone, isopropanol, n-butanol, methanol or combinations of these solvents with water.

v) Use and Optimization of the Amount of Ethanol-Water for Washing Procedures

In an experiment, the effect of concentration of ethanol in water was tested on washing of a known amount of RNA bound to beads. In a vessel, 5.5 μL of beads were added, washed, and re-suspended with Dynabeads® lysis buffer. Control Human Normal RNA (BioChain), was added to a concentration of 1 ng/μL. RNA was allowed to hybridize with the beads for 15 minutes, and 9 μL samples were placed in 11 vessels. All of the buffer was removed from these vessels. Next, 10% increments of ethanol in water were added to each vessel, from 0% to 100%, in a volume of 25 μL. Vessels were briefly vortexed and allowed to sit for 5 minutes. The ethanol was removed, and added once again. Then 2 μL samples were pipetted from each vessel into a qPCR plate, and the beads were allowed to dry at 60° C. for 15 minutes to remove all ethanol. RTPCR was then performed using the HPRT probe set and Verso one-step mix as described in Example 3.

The results, shown in the table below, showed that ethanol strips the oligo-dT beads of more 90%-100% of the mRNA when used at all concentrations below 80% ethanol in water. The optimal concentration of ethanol for preserving mRNA on beads was 90%. The use of solvents is especially useful for washing mRNA bound to oligo-dT beads in a massively parallel format.

| | Relative Number of mRNA Molecules |
|---|---|
| % of Ethanol in Water | (Normalized to 90% Ethanol Result) |
| 0 | 0 |
| 10 | 2.87 |
| 20 | 0.35 |
| 30 | 0.00 |
| 40 | 0.16 |
| 50 | 0.02 |
| 60 | 0.33 |
| 70 | 10.77 |
| 80 | 71.76 |
| 90 | 100.00 |
| 100 | 24.93 |

vi) Methods of degrading DNA

A study evaluated the potential of a DNase treatment to remove contaminating DNA by testing DNase compatibility with the Dynabeads® system because it was unknown if the RNA-DNA hybrids would survive DNase treatment. In each of six vessels, 20 μL of Dynabeads® lysis buffer was mixed with half of a scraped frozen mouse brain tissue section of 10 micron thickness, and was incubated for 10 minutes. Two control vessels had 4 μL of water-washed Dynabeads® beads added. To test whether a DNase treatment cleaves oligoDT moieties on Dynabeads, another two vessels had 4 μL Dynabeads® added, which were first washed with water, and incubated with 10 μL of DNase in buffer for 15 minutes followed by a 10 minutes 65° C. inactivation. To test whether a DNase treatment cleaves oligoDT molecules when hybridized with captured mRNA, the remaining two vessels had 4 μL of water-washed Dynabeads® added, and a DNase treatment after the following wash steps were complete. For all six vessels, wash treatment was performed after the addition of beads, including one wash with 125 μL Dynabeads® Wash A and a wash with 125 μL Wash B. Finally, all buffer was removed from the 6 vessels and 10 μL of 10 mM Tris-HCl was added. To evaluate the amount of mRNA in each of the six samples, 1 μL samples of elution buffer with beads, and 1 μL samples of the DNase buffer use to treat mRNA-oligoDT hybrids, were added to 10 μL of RT-PCR supermix, and duplicate samples were added to RT-PCR supermix without reverse-transcription enzymes to test for contaminating DNA, according to the manufacturer's protocol (Applied Biosystem Ag-Path ID), using the mouse HPRT and KCNJ1 probe sets described in Example 3. RT-PCR thermocycling was performed according to the manufacturer's protocol, using an AB 7500 real-time instrument.

The results showed that DNase treatment did not damage oligo-DT moieties on the DynaBeads. However, when mRNA was hybridized to the oligo-DT molecules, the DNase treatment eliminated more than 95% of the mRNA. The entirety of these mRNAs were recovered in the DNase buffer, showing that DNase treatment cleaved oligo-DT mRNA hybrids on beads. The results did not show any HPRT mRNA when reverse-transcriptase was not included, however, KCNJ1 DNA was detected. This shows a clear ability to accurately distinguish between DNA and mRNA in multiplex in a single vessel, and an ability to selectively eliminate DNA. These results also show that for the Dynabeads® system to be used in a single-vessel format, the DNase buffer needs to be heat-inacivated. Furthermore, the DNase treatment should be partially or totally dried to make room for PCR supermix. To prevent interference of DNase buffer with the salt concentration in PCR supermix, the DNase buffer should be replaced with water or very low concentrations of DNase buffer.

In an alternative embodiment, the concentration and type of salts in the nuclease buffer and type of nuclease may be chosen such that DNase activity is specific only for single-stranded or for double-stranded DNA. In particular, it is known that Nuclease P1 will not degrade double stranded DNA with 400 mM NaCl at pH 6.0 (Sigma Aldrich, St. Louis, Mo.). Also, Nuclease S1 will degrade only single-stranded DNA in the presence of zinc or calcium ions (Sigma Aldrich, St. Louis, Mo.).

### Example 22

### Methods to Wash Tissue in Multiwell Aluminum Plates

This example describes methods to wash a sample in a multi-well aluminum plates.

In this example, the step of removing unbound species from each vessel is simplified by washing the wells of a multi-well plate simultaneously in a large washing bath. The wells contain oligo-dT magnetic beads with mRNA hybridized to them. The three major challenges for performing a simultaneous wash are to ensure washing removes all lysis buffer to allow downstream detection; ensuring that there is no spreading of mRNA out of wells and into another; and ensuring that magnetic beads are not lost due to convective forces of rinsing.

To determine if a wash could be performed simultaneously, without crosstalk between positive and negative wells, an optimized mixture of 90% ethanol was used as a bath to purify wells in an aluminum multi-tier plate. Two aluminum plates were created as described in Armani et al., (Lab Chip, 9 (24): 3526-3534, 2009), with the permanent polymer film (McMaster-Carr FEP), but without the agarose and without the Kapton film. In each plate, 4 wells were filled with 1.8 μL of beads re-suspended in Dynabeads® lysis buffer containing 1 ng/μL of control human total RNA, and were allowed to hybridize for 5 minutes. Eight wells adjacent to the positive wells and 2 far away wells had beads added containing no RNA, to see if RNA spreads and can be captured during washing.

A Nalgene 5700 utility box (325 mL 13×7×6 cm) was modified to use as a washing bath for magnetic beads by taping a 100 lb force 3×3×1 cm neodymium magnet underneath the box using double-sided tape. To test a simple wash without agitation, the first aluminum plate was placed in the box, adhered to it with double sided acrylic tape, and magnetic beads were allowed to settle in the wells for 15 seconds. Then 90% ethanol was poured into the utility box, in an area far from the plate, to a volume of 200 mL. After 5 minutes, the ethanol was poured off and the plate was removed. For the second plate, treatment was identical to the first except that it was washed by agitation by placing the utility box containing the plate on a rocking shaker (Reliable Scientific Inc., model 55) at setting 90 for 5 minutes. Remaining ethanol in the wells were dried at 60° C. for 10 minutes. A reverse transcription reaction was performed to test only for mRNA bound to beads in the wells. The manufacturer's protocol was followed (Verso cDNA Kit), with the addition of 0.5% BSA in the reaction mix, and a volume of 1.8 μL was pipetted into each well. Samples were re-pipetted from each well to re-suspend beads and transferred to a qPCR plate. Samples were heated to 50° C. for 30 minutes and 95° C. for 2 minutes. 0.5 μL samples were then pipetted from each cDNA sample, and pipetted into a 10 μL PCR reaction for human HPRT using the Verso two-step kit. Samples were run for 60 cycles of PCR.

The results showed that no sample was detected in any of the wells of the first plate, indicating that the wash time or lack of agitation did not clean the lysis buffer in the wells. However, the second plate that was cleaned with agitation had a positive signal in the four wells with positive mRNA, while none of the negative wells gave any signal, even when amplified to 60 cycles. These results show that neither cross-over of beads or mRNA between wells was an issue in a batch wash. Because the binding of mRNA to beads was not near 100% efficiency, some mRNA would be expected in solution. It is proposed that the 90% ethanol prevents mRNA from spreading simply by precipitating the mRNA out of solution. The results also show that agitation is needed for washing to work in 5 minutes. At the volumes used, the fluid in the wells would be diluted by a factor of about 1000. It was also noted that while the external magnetic field keeps beads in the wells during washing, movement of beads was also significantly retarded by the presence of FEP sealing film and 90% ethanol. It was also noted that there was no contamination from re-using the plate.

### Example 23

### Dynabeads® Single Vessel mRNA Mapping from Frozen Tissue

The example illustrates the use the disclosed methods to both purify and detect nucleic acids in a single vessel format, and to use this technique in an array of miniature wells to create a map of gene expression across a tissue section.

In a study, tissue was pressed into a plate containing an array of wells and then in a single-vessel procedure the tissue is lysed, mRNA is captured and purified, and the targets are amplified.

mRNA specific to liver was detected from normal frozen mouse liver tissue sections (5 μm) in a 384-well plate.

First, three tissue sections were placed onto a film of silicone adhesive (Arseal 90697), which was then inverted onto a 384-well plate. Each section covered an area with 3 to 4 vials.

TABLE VII

| | | | | | |
|---|---|---|---|---|---|
| | | 9 | 9 | | |
| | | 01 | 12 | | |
| | | | | 05 | 7 |
| 07 | 5 | | | 07 | 08 |
| 05 | | | | | |

The mRNA was extracted by lysing the cells for 30 minutes using the commercial Picopure RNA Isolation Extraction Buffer (50% by volume guanidine thiocyanate, 22% by volume Triton X-100 surfactant, and an unknown amount of methylmercaptans (reducing agent)). The silicone film was removed. The mRNA was captured and purified using magnetic beads (Dynabeads®) functionalized with an oligo-dT moiety for mRNA capture. The use of beads prevented the capture of genomic DNA, which could cause false positive detection during PCR. After hybridizing the beads with mRNA from the crude lysate, the beads were washed four times to removed undesired tissue components and PCR inhibitors. Three of these washes were done with 10 mM Tris-HCl, 150 mM LiCl, 1 mM EDTA buffer and one with 10 mM Tris-HCl (pH 7.5). A neodymium bar magnet was used to hold the beads in place during the washes.

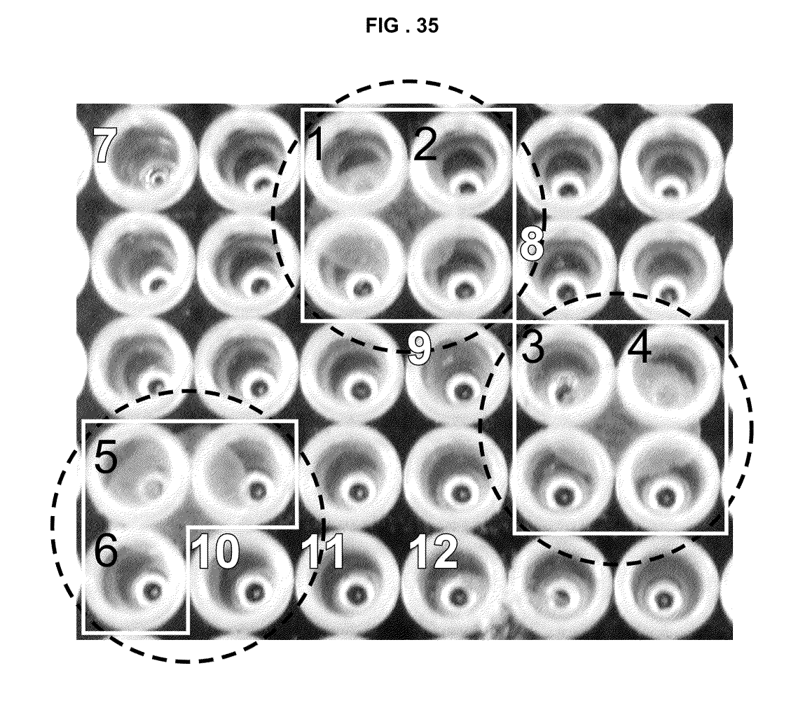

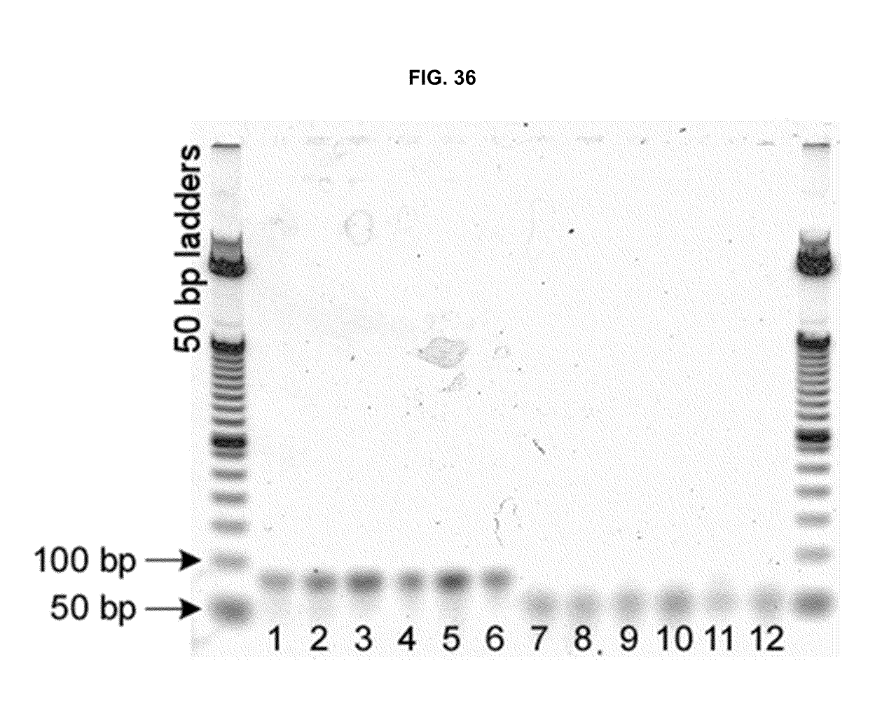

A real-time one-step reverse transcription PCR (RTPCR) was performed under standard conditions for the 74 bp mRNA target glycogen synthase 2 (GYS2), which is specific for the liver tissue. The plate was imaged every 10 cycles to monitor the progress of the reaction and to quantify the fluorescence levels in the wells. After 30 cycles a fluorescence signal appeared in the vials under the tissue (Table VII), but not in any of the other vials. To measure the relative fluorescent signal, the raw fluorescent averages for each well were obtained using the ImageQuant program, and the averages at 30 cycles were divided by those at 10 cycles. The amplification was confirmed by gel electrophoresis on products taken from 6 expected positive and 6 expected negative wells (FIG. 36). A product of the expected 74 bp was found in vials 1-6, which were under the tissue (see labels in FIG. 35), and no product of this size was found in vials that were not under the tissue. Even after 40 cycles of PCR, there was no apparent cross-contamination between wells.

This experiment has been performed several times, and shows that it is possible to transfer a tissue vertically into an array of wells, simultaneous isolating and preserving (without cross-contamination between wells) the positions of the mRNA for subsequent analysis. It also shows that it is possible to use a single-vessel procedure for mRNA extraction, purification, amplification, and detection. This protocol took about 5 hours, but this could be reduced to one hour by utilizing automation and plates with higher thermal conductivity. This experiment also demonstrates that mapping is possible using one of the most chaotropic lysis reagents, and that this chemistry can be made compatible with both hybridization on beads and downstream amplification by PCR. (Less aggressive lysis buffers leave tissue extra-cellular proteins intact, which nonspecifically bind to the magnetic beads. Such a buffer would inhibit PCR if even a small fraction of a percent were present, but the magnetically-based wash procedures dilutes any inhibitors by at least 80.000-fold.)

As with 2D-PCR for DNA, an entire process was crafted from beginning to end for mRNA. Both procedures shared the challenges of preserving tissue spatial locations, preventing crosstalk, preventing evaporation, extracting nucleic acids, carrying out PCR with designed primers sequences, and performing all reactions in parallel in individual wells. However, in addition to these, the mRNA map required the design of fluorescent molecular probes that enabled real-time PCR (to enable performing multiplexing with mRNA), and modification of the single-vessel process for working with the more sensitive mRNA, which needs to be protected from degradation.

### Example 24

### Dynabeads® Triplex mRNA & DNA Mapping from Frozen Tissue

This example provides methods for mapping multiple gene targets from multiple samples simultaneously in two dimensions.

To map three gene targets from three different mouse tissues simultaneously in two dimensions, a composite block of mouse liver, kidney, and heart was constructed by placing the organs in OCT. The composite tissue block was then sectioned to 10 micron thickness and placed onto a film of ARseal 90697. To remove the OCT and to stain the tissue for easy viewing, the film was placed in bath of 70% ethanol for 2 minutes, 100% eosin for 2 seconds, 70% ethanol for 30 seconds, and another 70% ethanol bath for 30 seconds to remove residual eosin stain. The film was then air dried for 2 minutes. A 384-well plate (Bio-Rad) was cut to an 8×8 size, and the inner 6×6 wells were filled with 4 μL of extraction buffer (PicoPure mRNA Kit). The tissue on the film was placed on to the 8×8 plate. It was compressed at 200 pounds of force under 50° C. heat for 1 minute to seal the film. The plate was then inverted to force the extraction buffer over the tissue. To fully digest the DNA, the tissue-side of the plate was heated for 16 hours at 50° C., while the wells were heated to 60° C. to prevent condensation. After the lysis, the plate was centrifuged to force fluid back into the bottom of wells. The sealing film was then heated to 100° C. for 2 minutes, and lifted off.

30 μL of oligo-DT Dynabeads® (Invitrogen) were decanted, and purified twice in 100 μL water before 600 μL water was added to the beads. 16 μL of the beads-water mixture was then added to each well with extraction buffer using a new pipette tip for each well. The plate was again sealed with sealing film (Applied Biosystems) and vortexed. After 1 minute, it was unsealed, and individual pipette tips were used to decant each well while the well was held over a strong magnet. If tissue was present, wells were re-pipetted to remove tissue but leave behind some beads. Afterwards, 10 μL of wash buffer B was added 3 times, with a wait period of 1 minute. A single pipette tip was used to add reagent, while a pipette tip connected to a vacuum was used to remove fluid. This was repeated for 10 μL of Tris HCl 10 mM as a fourth wash. Then, 5 μL of AgPath-ID one-step RTPCR mix, which included primers for GYS2, KCNJ1, and HPRT, was added to each well. The plate was sealed and subject to thermal cycling according to the manufacturer's protocol. During the cycling, the program was stopped and the plate was imaged at cycles 10, 20, 25, and 30 cycles.

The results showed strong detection of DNA at similar levels for GYS2 and KCNJ1. The results showed the stronger detection of HPRT mRNA. This shows that three targets, which can include mRNA and DNA, can be detected at different levels, from three different tissues, while preserving the spatial locations of the tissue. To achieve these results, the tissue was heated to about 50° C. In experiments not shown here, removal of the heating step eliminates most if not all of the signal from DNA.

Furthermore, these results showed that it is possible to use a single pipette tip connected to a vacuum to remove fluid from all of the wells during the wash procedure, without introducing any detectible cross contamination, as seen by noting zero detection in any of the wells that did not contain tissue. This allows the procedure to be performed much faster. An array of pipette tips connected to a vacuum manifold could be used to increase speed further.

| | | | | | | |
|---|---|---|---|---|---|---|
| KCNJ1 | 1.1 | 1.1 | 1.0 | 1.1 | 1.0 | 1.1 |
| | 1.3 | 2.5 | 1.8 | 2.9 | 1.1 | 1.1 |
| | 3.4 | 3.6 | 1.1 | 1.8 | 2.0 | 1.0 |
| | 3.2 | 3.8 | 1.1 | 1.8 | 1.5 | 1.0 |
| | 1.1 | 1.1 | 1.1 | 1.1 | 1.1 | 1.2 |
| | 1.1 | 1.1 | 1.1 | 1.1 | 1.0 | 1.0 |
| GYS2 | 1.0 | 1.0 | 1.0 | 1.0 | 1.0 | 1.1 |
| | 1.2 | 1.9 | 1.6 | 2.6 | 1.0 | 1.0 |
| | 2.0 | 2.1 | 1.0 | 2.4 | 1.7 | 1.0 |
| | 1.5 | 1.5 | 1.0 | 1.3 | 1.6 | 1.1 |
| | 1.1 | 1.0 | 1.0 | 1.0 | 1.1 | 1.0 |
| | 1.1 | 1.0 | 1.0 | 1.0 | 1.1 | 1.1 |
| HPRT | 1.2 | 1.2 | 1.1 | 1.2 | 1.2 | 1.2 |
| | 1.5 | 2.2 | 2.1 | 6.4 | 1.2 | 1.2 |
| | 3.5 | 6.7 | 1.2 | 3.6 | 4.9 | 1.1 |
| | 5.3 | 6.4 | 1.1 | 2.1 | 2.4 | 1.2 |
| | 1.2 | 1.2 | 1.1 | 1.2 | 1.2 | 1.2 |
| | 1.2 | 1.2 | 1.2 | 1.1 | 1.2 | 1.2 |

### Example 25

### ChargeSwitch® Single Vessel mRNA & DNA from Frozen Tissue

This example describes the intended use of the disclosed methods to both purify and detect total nucleic acids in a single vessel format, to detect mRNA or DNA from frozen tissue.

i. Methods to Simplify Nucleic Acid Isolation. The ChargeSwitch® manufacturer's protocol specifies several steps where samples are incubated for a period of time and then fluid is added or removed. Studies were performed to determine whether some of these steps could be simplified by combining reagents. In particular, the step of adding beads and buffer B9 was combined with the step of adding lysis buffer, and the step of adding buffer B9 was combined with the step of adding DNase. Furthermore, the recommended volumes are large, requiring a volume of 0.8 mL, and scaled down volumes were tested.

Frozen mouse kidney and mouse liver (Pel-Freez Biologicals) were sectioned to 10 micron thickness and placed on a glass slide; a serial recut was made of the two tissues and all of the following reactions were performed on duplicate tissues using recuts. One half of each tissue section was generally processed according to the manufacturer's protocol except that all volumes were scaled down by 20-400 fold, and are specified below. Each tissue half was placed into solution containing ChargeSwitch® lysis buffer, 0.2 mg/mL proteinase K and 5 mM Dithioerythritol, in a total volume of 14 μL. The samples were incubated at 60° C. for 15 minutes. Then, 0.5 μL Chargeswitch® beads and 5.75 μL buffer B9 were added. Then, the samples were washed with 20 μL for W14. These samples were then split into two replicate tubes of equal volume, and one set of these tubes was further processed according to the recommended protocol. 6.25 μL of DNase diluted in 1× buffer was added to the tubes and incubated for 15 minutes, and then 2 μL of buffer B9 was added. Samples were then washed with 19 μL of W13, 12.5 μL of W14, and eluted in 10 μL of elution buffer. For the other set of replicate tubes, samples were processed identically except that DNase in 1× buffer already contained the buffer B9.

For the other half of each tissue section, samples were processed as described except that the initial lysis buffer contained the Chargeswitch® lysis buffer, 0.2 mg/mL proteinase K and 5 mM Dithioerythritol, in a total volume of 14 μL, and also contained the 0.5 μL beads and 5.75 μL buffer B9. At the part were samples were replicated, the replicates were discarded.

Finally, samples were amplified in yet another shortened protocol. Instead of eluting the mRNA off of beads, a 1 μL sample of the beads were used directly from the elution buffer. Samples were pipetted in duplicate wells containing RTPCR SuperMix which contained 5 μL SuperMixSuperMix and 0.4 μL enzyme mix from AgPath-ID™ (Applied Biosystems), 0.25 μL of each KCNJ1, GYS2, and HPRT probe mixes described earlier, and water up to 10 μL total volume. Samples were thermocycled at 50° C. and 95° C. for 10 min each, and 60 cycles of 95° C. for 15 seconds, 60° C. for 30 seconds using an Applied Biosystems 7500 real-time instrument. The results showed that there was no difference for either mouse liver or kidney tissue when using the standard protocol or the protocol which combines the lysis, beads, and B9 buffer as a single step. This cuts out a significant step. However, when combining the DNase step with buffer B9, samples were observed to chimp, and some of these samples detected DNA. However, a few samples still showed no DNA detection. This shows that it may still be possible to shorten the DNase procedure with further optimization, such as adding more DNase enzyme or reducing the amount of buffer B9 added, possibly down to zero.

In conclusion, the studies demonstrate that significantly fewer steps and less reagents can be used than is currently known. The method reduces the need for up to 3 major pipetting and processing steps. The volumes used were most frequently 40 times less than the recommended amount, but the amount of beads used were 400 times less than the recommended amount.

ii. Modifications to Enable Nucleic Acid Isolation with GITC buffer

The Chargeswitch® beads system was designed for isolations of cells that are freshly prepared, and there are no protocol for using the highly chaotropic GITC buffer with this system on archival samples. To this end a protocol was developed for using the PicoPure extraction buffer, which contains 50% GITC and 22% Triton X-100. First, frozen mouse brain sections were scraped into three vessels. Vessel 1 contained 50 μL Chargeswitch® lysis buffer. 40 μL of PicoPure extraction buffer with 4 μL sodium acetate pH 5.2 was added in vessel 2. 10 μL of PicoPure extraction buffer was in vessel three. All three vessels were incubated at 60° C. for 15 minutes. For all vessels, 10 μL of Chargeswitch® beads was added. The first vessel also had 20 μL of Chargeswitch® Buffer 9 added, and the third vessel also had 30 μL of water added. All vessels were then washed with 50 μL buffer W14, 25 μL DNase treatment, 75 μL buffer W13, and 50 μL buffer W14, and re-suspended in 15 μL of elution buffer according to the Chargeswitch® beads protocol. 5 μL samples of each vessel were placed in a qPCR plate, the beads were decanted, and 10 μL of RT-PCR supermix was added according to the AgPath-ID protocol, with probe sets for mouse HPRT and KCNJ1 added.

The results showed similar levels of amplification when using the Chargeswitch® beads system and when using the PicoPure extraction buffer with sodium acetate added, within 1-2 cycles of amplification. This shows that the PicoPure extraction buffer is compatible with Chargeswitch® beads in a single-well protocol and could be used on archival tissues or on tissues which contain high levels of ribonucleases.

### Example 26

### ChargeSwitch® Duplex mRNA mapping from Frozen Tissue

This example describes the use of an array of wells to create a map of gene expression for two genes across a composite of three different tissues. A mouse-specific gene was detected in two tissues, a mouse kidney-specific gene was detected in one tissue, and no gene was detected from the non-specific chicken tissue.

i. Making and Preparing the Composite Tissue Section. A composite of three tissues was made by placing whole mouse brain, chicken thymus, and mouse kidney (Pel-Freez Biologicals) in a container and filling the space between tissues with O.C.T. compound (Tissue-Tek 4583). This composite was then sectioned to 10 micron thickness and placed onto a film of ARseal 90697 (Adhesives Research). It was then thawed at 50° C. for 1 min. This film was then placed in a bath of 70% ethanol for 2 minutes to remove the O.C.T., a bath of 5% Eosin Y and 95% ethanol for 60 seconds to stain the tissue, and two baths of 70% ethanol for 30 seconds each. Tissue was then dried and fixed to the adhesive by heating it to 95° C. for 15 minutes.

ii. Methods for Adding a Protein Denaturing Agent and Providing a Vessel.

ChargeSwitch® lysis buffer master mix was made (560 μL Lysis buffer mix, 2.8 μL 1M DTT (Sigma 43816), and 5.6 μL Proteinase K) and 14 μL was added to 25 wells in a 5×5 grid across a 384-well plate (soft clear polypropylene plate, Bio-Rad) cut into an 8×8 piece. Any residual fluid on top of wells was removing by wiping with WypA11™

iii. Methods for Adding a Tissue Sample and Sealing the Vessel. To transfer the tissue, the film was placed over the plate and was pressed with a blunt marker edge to partially seal the film.

The film was then covered with a red silicone sheet approx 2 mm thick to evenly distribute pressure, and placed in a PCR thermocycler as previously described for mapping DNA, except that a 384-well plate block alpha unit with heated lid was used. The heated lid was set to 75°, and was turned ⅔^{rd} turns of pressure. The previously used rig including Plexiglas® and aluminum block were placed on top of the heated lid. 150 pounds of additional pressure was added with the compression rig. The temperature of the red silicone on the inner side of the heated lid was verified to within 1° C. using an infrared thermometer.

After 2 minutes, the plate was removed, and cooled for 1 min. The plate was inverted 5 times to distribute lysis fluid throughout the wells. The last flip distributed fluid over tissue. This plate was then taken (upside down) and placed on the 384-well alpha block onto a thin sheet of stainless steel. The other side of the plate was covered with silicone and sealed with the heated lid to ⅓^{rd} turn of pressure (no compression rig beyond this point). The tissue-side of the plate was heated to 60° C. to incubate the lysis buffer, and the lid (bottom of plate that is upside down) was set to 75° C. to prevent condensation. This setup was incubated for 15 min.

iv. Methods for Adding a Nucleic Acid Precipitant. After the digestion, the mixture was vortexed and centrifuged at 3000 rpm for 2 minutes. The tissue and film were then heated off at 90° C. for one minutes with ⅔^{rd} turn lid pressure. Then a mixture of 0.5 μL Chargeswitch® beads and 5.75 μL Chargeswitch® Buffer B9 was added per tube—from a solution of 20 μL beads and 230 Chargeswitch® Buffer B9 SuperMix. The buffer B9 is acidic, and caused the beads to have a negative charge for binding nucleic acids. New tips were used for each well. A single repipette/plunge was used to aid in mixing, preventing the need for another seal, vortex and centrifuge step. The sealing film was removed as previously described.

v. Methods for Removing Unbound Species from the Vessel. After hybridization for 5 minutes, the plate was placed over 4 4 mm diameter cylindrical magnets arranged vertically. This allowed decanting of 10 wells at a time. A new pipette tip was used for each well to remove the solution by pipetting out fluid. Then new pipette tips were used to add 12.5 μL of W14—from 500 μL stock W14. Solution was not individually repipetted—the plate was heat sealed at 90° C. for one minute with an ABI qPCR Plate sealing film, cooled for 1 minute, vortexed, flipped several times, and left to hybridize for 1 minute before a 3000 rpm 2 minute centrifuge step. The sealing film was then removed as before. Each well was individually decanted on magnets as before.

vi. Methods for Degrading DNA. 6.25 μL DNase mix was added to each tube. DNase is made with 5 μL DNase and 250 μL DNase buffer. The plate was sealed and flipped 5 times. This was incubated for 10 minutes at room temperature. Then, the plate was centrifuged for 3000 rpms, 2 minutes and sealing film removed by applying heat. Then, 2 μL of B9 was added to each tube (from 80 μL stock B9), using new pipette tips and repipetting 3 times. 2 minutes was allowed for sufficient time for RNA to bind to the beads again. Wells were decanted as before.

vii. Methods for Removing Unbound Species from the Vessel and Enable Downstream

PCR. Next, solutions had 19 μL of W13 (from 760 μL stock W13) added, waiting 2 minutes, and then decanting, again using new pipette tips for each well. Next, solutions were washed with 12.5 μL W14 (from 500 μL stock W14) by sealing, vortexing, flipping, waiting 5 min, centrifuging, heating off the sealing film, and then decanting. This last decanting step made it possible to perform PCR directly on the beads without an elution step.

viii. Methods for Detection and Mapping. PCR mix was added to all wells in a volume of 5 μL from a SuperMix of 100 μL PCR buffer and 8 μL enzymes from AgPath-ID™ (Applied Biosystems), 5 μL of each KCNJ1 and HPRT probe sets described in the Example 3, and water up to 200 μL. PCR was added to all 25 wells using new tips for each well. Samples were thermocycled at 50° C. and 95° C. for 10 min each, and 60 total cycles of 95° C. for 15 seconds, 60° C. for 30 seconds using the PTC-200 thermocycler and 384-well block, with heated lid set to 100° C. and ⅔^{rd} lid pressure. At the end the RT step and cycles of 20, 25, 30, 40, and 60, samples were imaged on the Typhoon Imager for detectors CY5 and FAM.

These studies showed a detection signal for the mouse control mRNA HPRT1 from both brain and kidney tissues by 30 cycles, while the well containing chicken thymus nucleic acids showed no detection even up to 60 cycles of PCR. The results also showed the simultaneous detection of a second signal for the mouse kidney mRNA KCNJ1 within 30 cycles of PCR only for kidney tissues, but not for mouse brain or chicken thymus tissues.

This study demonstrates that it is possible to transfer tissues sections into a multi-titer plate and to preserve the 2D layout of the tissue. The technique took about 5 hours to perform, and represents a major reduction of time needed to isolate and analyze many tissue sub-regions, making it practical to perform such an analysis on a routine basis. Furthermore, because the tissue is applied to a grid, it improves the accuracy and consistency of sequestering tissue into individual regions of equal size and distance. By staining the tissue pink with a relatively inert eosin dye, it was possible to see the effectiveness and approximate level of elution of molecules out of the cells. The use of non-specific tissue in this experiment provides internal references that can replace “no reverse-transcriptase” controls. In this experiment, the non-specific-species tissue was chicken thyroid. The non-specific mouse tissue was mouse brain tissue, which does not express kidney mRNA.

Amplifying two targets at once, one general for the species and one specific for the organ, provides an internal reference for normalizing the abundance of the target gene to the species-specific gene (e.g. the amount of tissue in the wells) and for compensating for amplification efficiency Finally, the technique can be used for miniaturization, for example to 1536 well PCR plates (produced by KBioscienes), and to robotic automation.

It was noted that the sealing film should be evenly sealed at all times when being compressed to reduce crosstalk, such as during the initial lysis. Furthermore, too much tissue per well consistently reduced the amplification yield. It was noted when performing the experiments that wells with the most tissue contained the most clumping. This indicates that non-target tissue components, such as genomic DNA, may be competitively inhibiting the binding of the desired mRNA. In some cases this may be useful.

### Example 27

### Methods and Construction of Devices to Filter Tissues Upon Transfer

This example provides methods and devices for filtering of tissue upon transfer to a disclosed substrate.

i. Construction of 384-well plates with stainless steel mesh embedded in the top surface of the plate. It may be desirable to filter the tissue through a sieve or mesh during the transfer step to reduce the size of tissue fragments and to reduce the maximum amount of tissue in each well. Substrates were constructed by melting the tops of polypropylene 384-well plates into a stainless steel mesh with a sieve size of 37.5 microns. To achieve this, clear 384-well plates with hard shell skirt (Bio-Rad) were placed in a holder (384-well block alpha unit, Bio-Rad) to prevent movement of the wells. A stainless steel type 316 mesh (McMaster Carr 9319T46) was placed over the 384-well plate. This mesh was then covered with 25.4 micron thick FEP fluoropolymer (McMaster 85905K62) to serve as a release layer. This sandwich of plate, mesh, and film was covered with a plate with a temperature of about 130° C. for about 2 minutes. After letting the plate cool, the release layer was removed and the 384-well plate was cut into smaller 8×8 sized plates.

ii. Methods for using 384-well plates with embedded mesh. An 8×8 sized plate with embedded mesh was used to map the composite tissue of Example 26 as described in Example 26, with two modifications. First, the to fill the wells with lysis solution, a 10XL pipette tip (Neptune) was used, pressing the pipette tip evenly against the mesh with about 5 pounds of force, waiting for 3 seconds, and then quickly pressing the pipette. Fluid filled in this manner settled in the bottom of the wells, except for a small amount of fluid that remained in the center of the filter mesh. To settle that fluid, the plate was centrifuged at 3000 rpm for 3 minutes. Alternatively, fluid could have been filled before embedding the mesh. The second modification was puncturing the stainless steel mesh to provide access to the well after the tissue transfer, lysis, and centrifugation step. This was done by cutting a 6×5 grid out of a new 384-well plate and stacking the new plate on top of the mesh. This sandwich was placed inside the 384-well plate block of the PCR machine, and the lid was closed. Pressure on the lid was increased until all of the meshes were punctured. All other steps were the same.

These results were similar to the results for not using a mesh. However, because the mesh was used, tissue was much more easy to handle and re-suspend during the initial lysis and wash steps. Furthermore, because the wells with the most tissue were not as overloaded as before, these wells gave greater signal than expected. The downside of using the mesh was that one well leaked, yielding 4 false positive signals. In the future, slight modifications such as the use of a 384-well plate of single polymer composition may prevent the formation of such leaks.

As an alternative embodiment to the use of a stainless steel mesh, one could overlay a 384-well plate with a pure adhesive film, such as transfer film. The transfer film could be between 50-250 microns in thickness, with very strong adhesion, and good weatherability. The transfer film could also have a matrix of holes between 10-100 microns in diameter. The transfer film could also have holes that are hydrophilic. The transfer film could have an open are that can be controlled, between 5% to 50%. An example of such a transfer film is the ARseal 9020 from Adhesive Research.

### Example 28

### Mapping DNA Methylation

This example provides mapping DNA methylation by utilizing the disclosed methods. DNA methylation can be mapped by applying the methods described herein by using magnetic beads that can capture methylated DNA. In this example, tissue is transferred into a 384-well plate as described previously. The plate will already contain proteinase K buffer described in Armani et al. (Lab Chip, 9 (24): 3526-3534, 2009), and is sealed after the transfer step. Tissue is then incubated for 16 hours (overnight) at 65° C., and 5 minutes at 95° C., but without any agarose and using a volume of 15 μL, to lyse the tissue. A DNA restriction digestion enzyme, such as HaeIII, and the associated buffer specified by the manufacturer (New England Biolabs R0101L), are added to the wells at a volume of 5 μL, but the concentration of this solution is 4-fold greater than recommended. Plates are sealed with film as previously described (Applied Biosystems Sealing Film, 105° C.), and incubated at 37° C. overnight to cleave DNA. Plates are vortexed periodically during this process. The restriction enzyme is then heat inactivated at 80° C. for 20 minutes. If the enzyme cannot be heat inactivated, it should be washed by the next step. In the next step, 1 μL of magnetic beads with CpG island methylation binding protein is added to each well (MethylMagnet® Riobomed). The beads are periodically vortexed and incubated for 1 hour to hybridize with methylated DNA fragments. The DNA is then purified using the wash steps as specified by the manufacturer. Some minor optimizations, such as the wash buffer concentration and number of washes, may be needed. Finally, the initial proteinase K step is repeated with only a 30 minute incubation and 2 minute inactivation to elute DNA from the bead-protein complex. The mixture containing DNA is then dried at 95° C. for 30 minutes. Finally, PCR mix is added containing primers that correspond to the methylated region of interest, cycled, and analyzed as is known in the art. As a control, the entire procedure may be repeated by using a different restriction nuclease that is specific to DNA methylation and would degrade the target DNA fragment.

### Example 29

### Mapping Micro RNA or Other Small RNA

In this example, micro RNA is mapped by applying the methods described herein.

The tissue of interest is first transferred into a 384-well plate containing Chargeswitch® beads and Chargeswitch® lysis buffer as previously described. After performing the lysis procedure as described, the beads are purified and a DNase treatment is applied. After the DNase is inactivated, the beads are further washed, all buffer is removed, and the beads are dried at 50° C. for 15 minutes. The novel part of this procedure is the next step, where microRNA is converted to cDNA and then amplified in a single vessel format across the array of wells. First, RT primer, for Mir-21 for example (Applied Biosystems, hsa-miR-21 Assay), is added to the reverse transcription mix (Appled Biosystems TaqMan® Micro RNA Reverse Transcription Kit) according to the manufacturer's recommended protocol, and pipetted into each well at a volume of 2 μL. The plate is cooled to 16° C. for 30 minutes, heated to 42° C. for 30 minutes, and 85° C. for 5 minutes. Finally, PCR mix, containing the miR-21 Taqman Probe set, is pipetted into each well at a volume of at least 18 μL. Plates are then thermally cycled and products detected as is known in the art.

### Example 30

### Mapping Electrophoresis Gels

The example provides methods for mapping electrophoresis gets.

The identity or quantity of nucleic acids from an electrophoresis gel is desired, particularly from small amount of samples. In an example first step, mRNA is purified from human tissue and converted to cDNA. Next, the cDNA is run on a 2% agarose electrophoresis gel to separate bands with sizes from 200 to 2000 base pairs. The gel includes a ladder, which is post-stained with Eva-Green dye. Next, the gel is placed on top of a 384-well plate that already contains PCR amplification reagents. The reagents may be concentrated such that combination with the water in the gel results in the desired PCR concentration. The gel is secured by taping it in place and centrifuged at 4000 rpm for 5 minutes to drive the gel into the wells of the plate. The gel is melted by heating to mix the agarose with the PCR reagents. The plate is thermocycled to amplify Beta Actin target cDNA, as is known in the art. By identifying the positions of detected molecules, or their time of detection, significant information can be obtained, such as the amount of starting material, the size of the molecules, and the presence of mutations.

### Example 31

### Removal of Magnetic Beads

The example provides methods that can be used to remove magnetic beads.

If the need arises to remove magnetic beads but not the captured targets, because of incompatibility with later procedures or a desire to switch to another bead type, the magnetic beads are removed from the vessels. In this example, DNA is first captured onto Chargeswitch® beads and purified as previously described. In order to remove the beads, the plate is heated to 80° C. for 10 minutes. The sealing film is removed, and a disposable magnetic manifold that fits inside the 384-well plates is provided above the wells. The magnetic manifold can be placed such that it is about a millimeter above the fluid, so that the magnetic beads are drawn out of solution quickly.

In view of the many possible embodiments to which the principles of the disclosed invention may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method for transferring, isolating and amplifying nucleic acids from a two-dimensional (2D) biological sample within a single device while maintaining the 2D spatial relationship between the nucleic acids that was present in the original 2D biological sample, comprising:
providing the 2D biological sample to the single device, which device comprises a substrate having a plurality of through-holes, wherein each through-hole comprises a first opening on a first face of the substrate and a second opening on a second face of the substrate thereby forming a through-hole;
transferring portions of the 2D biological sample into the plurality of through-holes of the device;
providing conditions sufficient to free nucleic acids from the transferred biological sample portions within the plurality of through-holes of the device; and
amplifying target nucleic acids by polymerase chain reaction in the presence of a surface coating and amplification reagents, wherein the surfactant is added prior to the amplification reagents, thereby amplifying target nucleic acids while preserving the 2D spatial relationship of the target nucleic acids relative to their original position in the original 2D biological sample throughout the method in a single device.

2-25. (canceled)

26. A method for purifying nucleic acids from a biological sample within a single vessel, the method comprising:
providing a vessel comprising polypropylene, polyethylene, polystyrene, polycarbonate, fluoropolymer, acrylic, aluminum, stainless steel, ceramic, silicone, silicon, acrylic adhesive resin, or silicone adhesive resin;
providing a nucleic-acid binding surface to the same single vessel, the nucleic-acid binding surface comprising silica, silicon, silicon carbide, silicon nitride, metal oxides, polycarbonate, polystyrene, nitrocellulose, cellulose, or chitosan;
adding into the same single vessel a biological sample comprising nucleic acids;
adding into the same single vessel at least 1% Triton X-100, Tween 20, or alkali dodecyl sulfate, and guanidinium isothiocyanate;
allowing sufficient time to elapse to free the nucleic acids from the biological sample;
adding to the vessel a blocking agent comprising bovine serum albumin, poly(ethylene glycol), polyvinylpyrrolidone, Tween 20 or a combination thereof;
adding a nucleic acid precipitation agent to the vessel; and
removing unbound species from the vessel, thereby purifying nucleic acids from a biological sample in a single vessel.

27.-36. (canceled)

37. The method of claim 26, wherein the nucleic acid binding surface is the vessel, a silica filter, silica beads, or silica powder.

38.-71. (canceled)

72. A method for purifying nucleic acids from a sample within a single vessel, the method comprising:
adding a sample and a lysis agent into the vessel having a binding surface with a negative charge;
adding into the vessel a nucleic acid precipitation agent;
removing unbound species from the vessel by rinsing with a washing agent, wherein the washing agent comprises a solvent and a salt, and the salt has a concentration of at least about 100 mM; and
adding a blocking agent into the vessel, wherein bound species are placed into a state that permits subsequent manipulation or detection.

73. (canceled)

74. The method of claim 72, wherein the vessel has two openings, whereby fluid can be flushed through the vessel.

75. The method of claim 72, wherein the binding surface comprises:
an oxide, a semiconductor, a polymer, polycarbonate, polystyrene, nitrocellulose, or chitosan.

76. The method of claim 72, wherein the binding surface comprises silica, silicon, silicon with a native oxide, silicon carbide, silicon nitride or a metal oxide.

77-78. (canceled)

79. The method of claim 72, wherein the solvent comprises methanol, ethanol, n-butanol, acetone, or isopropanol.

80. The method of claim 72, wherein the blocking agent is bovine serum albumin, poly(ethylene glycol), polyvinylpyrrolidone, Tween 20, or a combination thereof.

81. The method of claim 72, wherein the binding surface is a bead having a pH-dependent surface charge, the charge being positive or negative, and the blocking agent having a pH sufficient to change the charge.

82. The method of claim 72, further comprising adding DNase to the vessel, whereby DNA is degraded.

83. A method for preparing nucleic acids from a tissue within a single vessel, comprising:
adding a tissue sample and a lysis agent into the vessel having a nucleic-acid binding surface;
precipitating nucleic acid in the vessel to create bound nucleic acids; and
removing any unbound species from the vessel by rinsing with a washing agent.

84. The method of claim 83, further comprising vortexing the contents of the vessel after adding the tissue sample and the lysis agent into the vessel.

85. The method of claim 84, wherein the nucleic acid binding surface comprises an oligonucleotide, a protein nucleic acid, a locked nucleic acid, a poly-dT nucleic acid, or a magnetic or paramagnetic bead having a nucleic acid surface.

86. (canceled)

87. The method of claim 84, wherein the lysis agent comprises guanidinium isothiocyanate at a concentration of at least about 25% by volume of the vessel's fluid contents and Triton X-100 at a concentration between about 11% and 22% by volume of the vessel's fluid contents.

88. (canceled)

89. The method of claim 84, wherein the nucleic acids are precipitated by a precipitation agent; and the precipitation agent comprises water in an amount sufficient to dilute the guanidinium isothiocyanate to between about 2.5% and 20% by volume of the vessel's fluid contents.

90. (canceled)

91. The method of claim 84, wherein the rinsing agent is approximately 87% to 95% ethanol.

92. The method of claim 84, further comprising:
amplifying nucleic acids in the same single vessel following purifying nucleic acids.

93.-99. (canceled)

100. The method of claim 72, wherein the biological sample is a tissue sample.

101. The method of claim 72, further comprising amplifying or detecting the nucleic acids.

102. The method of claim 72, further comprising sealing the vessel, thereby substantially preventing evaporation of the fluids.
